SEQUENCE LISTING (48 sequences)

SEQ ID NO: 1 - DNA, 363 bp, Artificial Sequence
  Synthetic polynucleotide
gaagtgcagctgctggaatccggcggaggcctggtgcagcctggcggctctctgagactgtcctgcgccgcctccgagttcaccttcggcagctgcgtgatgacctgggtccgacaggctcccggcaagggcctggaatgggtgtccgtgatctccggctccggcggctccacctactacgccgactctgtgaagggccggttcaccatctcccgggacaactccaagaacaccctgtacctgcagatgaactccctgcgggccgaggacaccgccgtgtactactgcgccaagaccccctggtggctgcggtcccccttcttcgattactggggccagggcaccctggtgacagtgtcctcc

SEQ ID NO: 2 - protein, 121 aa, Artificial Sequence
  Synthetic polypeptide
EVQLLESGGGLVQPGGSLRLSCAASEFTFGSCVMTWVRQAPGKGLEWVSVISGSGGSTYYADSVKGRFTISRDNSKNTLYLQMNSLRAEDTAVYYCAKTPWWLRSPFFDYWGQGTLVTVSS

SEQ ID NO: 3 - DNA, 363 bp, Artificial Sequence
  Synthetic polynucleotide
caactgttgctccaggaatccggtcctggtcttgtaaagccatctgagactctctcccttacctgtaccgttagcggaggaagtctttcctcaagcttctcctactgggtgtggatcagacagcctcccggaaaagggttggagtggattggcacaatatactactccggcaacacttactataaccccagcctgaagagcaggctgactatctctgtcgacaccagtaaaaatcacttttctctgaatctgtcttcagtgaccgcagccgacaccgccgtgtattattgcgctcggcgcgccgggattctgacaggctatctggattcatggggccaggggacattggttacagtgtctagt

SEQ ID NO: 4 - protein, 121 aa, Artificial Sequence
  Synthetic polypeptide
QLLLQESGPGLVKPSETLSLTCTVSGGSLSSSFSYWVWIRQPPGKGLEWIGTIYYSGNTYYNPSLKSRLTISVDTSKNHFSLNLSSVTAADTAVYYCARRAGILTGYLDSWGQGTLVTVSS

SEQ ID NO: 5 - DNA, 363 bp, Artificial Sequence
  Synthetic polynucleotide
cagctgctgctgcaagaatccggccctggcctggtgaaaccctccgagacactgtccctgacctgcaccgtgtccggcggctccctgtcctccagcttctcctactgggtctggatccggcagccccctggcaagggcctggaatggatcggcaccatctactactccggcaacacctactacaaccccagcctgaagtcccggctgaccatctccgtggacacctccaagaaccacttcagcctgaagctgtcctccgtgaccgccgctgacaccgccgtgtactactgtgccagaagggccggcatcctgaccggctacctggactcttggggccagggcaccctggtgacagtgtcctcc

SEQ ID NO: 6 - protein, 121 aa, Artificial Sequence
  Synthetic polypeptide
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSS

SEQ ID NO: 7 - DNA, 354 bp, Artificial Sequence
  Synthetic polynucleotide
caggtgcagctcgtgcagtccggcgcagaggtcaaaaagcctggtgcatctgtgaaagtgagttgcaaggctagcggctacacctttaccggatgttatatgcattgggtacgccaagcccccggacaaggcttggaatggatggggcgtatcaacccaaactctggcgggactaattacgcccagaagtttcagggaagggtgactatgacaagggacacatccatatccaccgcttatatggacctgtctcgactgcggtctgatgatacagccgtttattactgcgccagagactacagcggatggagcttcgattattgggggcagggtactttggtcacagtttcaagt

SEQ ID NO: 8 - protein, 118 aa, Artificial Sequence
  Synthetic polypeptide
QVQLVQSGAEVKKPGASVKVSCKASGYTFTGCYMHWVRQAPGQGLEWMGRINPNSGGTNYAQKFQGRVTMTRDTSISTAYMDLSRLRSDDTAVYYCARDYSGWSFDYWGQGTLVTVSS

SEQ ID NO: 9 - DNA, 369 bp, Artificial Sequence
  Synthetic polynucleotide
cagctgcagttgcaggagtcaggccccggtttggttaagccttctgaaaccctttctctcacatgcacagtatccggtggctccatctccagttcaagttactactggggatggatccggcaacccccaggaaaagggctggagtggattggcaatatatattactctgggtccacctattacaacccttccctgatgagtagagtgaccatcagcgtggacacaagcaaaaaccaattcagcctgaagctttctagcgtgaccgctgccgacacagctgtctattactgtgcccgccagcttagttataactggaatgataggctgtttgattactggggccaggggactctcgttacagtcagcagc

SEQ ID NO: 10 - protein, 123 aa, Artificial Sequence
  Synthetic polypeptide
QLQLQESGPGLVKPSETLSLTCTVSGGSISSSSYYWGWIRQPPGKGLEWIGNIYYSGSTYYNPSLMSRVTISVDTSKNQFSLKLSSVTAADTAVYYCARQLSYNWNDRLFDYWGQGTLVTVSS

SEQ ID NO: 11 - DNA, 357 bp, Artificial Sequence
  Synthetic polynucleotide
tcttacgtgctgacccagcccccctccgtgtctgtggctcctggccagaccgccagaatcacctgtggcggcaacaacatcggctccacctccgtgcactggtatcagcagaagcccggccaggcccccgtgctggtggtgcacgacgactccgaccggccttctggcatccctgagcggttctccggctccaactccggcaacaccgccaccctgaccatctccagagtggaagccggcgacgaggccgactactactgccaagtgcgagactcctcctccgaccacgtgatcttcggcggaggcaccaagctgaccgtgctgggccagcctaaggccgctccctccgtgaccctg

SEQ ID NO: 12 - protein, 119 aa, Artificial Sequence
  Synthetic polypeptide
SYVLTQPPSVSVAPGQTARITCGGNNIGSTSVHWYQQKPGQAPVLVVHDDSDRPSGIPERFSGSNSGNTATLTISRVEAGDEADYYCQVRDSSSDHVIFGGGTKLTVLGQPKAAPSVTL

SEQ ID NO: 13 - DNA, 357 bp, Artificial Sequence
  Synthetic polynucleotide
tcttacgtgctgacccagtccccctccgtgtccgtggctcctggccagaccgccagaatcacctgtggcggcaacaacatcggctccaagtccgtgcactggtatcagcagaagcccggccaggcccccgtgctggtggtgtacgacgactccgaccggccctctggcatccctgagcggttctccgcctccaactccggcaacaccgccaccctgaccatctccagagtggaagccggcgacgaggccgactactactgccaagtgtgggactcctcctccgaccacgtggtgttcggcggaggcaccaagctgaccgtgctgggccagcctaaggccgctccctccgtgaccctg

SEQ ID NO: 14 - protein, 119 aa, Artificial Sequence
  Synthetic polypeptide
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTL

SEQ ID NO: 15 - DNA, 357 bp, Artificial Sequence; identical to SEQ ID NO: 13
  Synthetic polynucleotide

SEQ ID NO: 16 - protein, 119 aa, Artificial Sequence; identical to SEQ ID NO: 14
  Synthetic polypeptide

SEQ ID NO: 17 - DNA, 351 bp, Artificial Sequence
  Synthetic polynucleotide
gacatccagatgacccagtcaccatcatccctttccgcatctgtcggagatagagtgactatcacctgcagggcttctcaaggtatttccaactacctcgcctggttccagcaaaagccaggtaaagccccaaagagcttgatctacgccgcttctagtctgcagagtggagttcctagtaagttctccggctctggcagtggcacagattttaccttgaccatttccagcctgcagtctgaggatttcgctacctactattgtcagcagtatgacagctatccccccacatttggggggggcactaaggtggagataaaacggacagtggctgccccttctgtctttatt

SEQ ID NO: 18 - protein, 117 aa, Artificial Sequence
  Synthetic polypeptide
DIQMTQSPSSLSASVGDRVTITCRASQGISNYLAWFQQKPGKAPKSLIYAASSLQSGVPSKFSGSGSGTDFTLTISSLQSEDFATYYCQQYDSYPPTFGGGTKVEIKRTVAAPSVFI

SEQ ID NO: 19 - DNA, 351 bp, Artificial Sequence
  Synthetic polynucleotide
agctatgagctgacccagcctctgagcgtatctgtcgctctcggccagacagccagaattacctgtggcggcaataacataggatccaaaaatgttcactggtatcagcaaaaacctggccaagctcccgtgctcgtgatctaccgggactctaaccgacccagtggaatccccgaacgctttagcggttccaactctggaaatacagctactctgactatctccagggctcaggccggggatgaggccgattactactgccaggtgtgggactcaagcacagtggtcttcggcggaggtaccaagttgactgttcttgggcagccaaaggccgcaccttcagtgaccctg

SEQ ID NO: 20 - protein, 117 aa, Artificial Sequence
  Synthetic polypeptide
SYELTQPLSVSVALGQTARITCGGNNIGSKNVHWYQQKPGQAPVLVIYRDSNRPSGIPERFSGSNSGNTATLTISRAQAGDEADYYCQVWDSSTVVFGGGTKLTVLGQPKAAPSVTL

SEQ ID NO: 21 - protein, 5 aa, Artificial Sequence
  Synthetic polypeptide
SCVMT

SEQ ID NO: 22 - protein, 17 aa, Artificial Sequence
  Synthetic polypeptide
VISGSGGSTYYADSVKG

SEQ ID NO: 23 - protein, 12 aa, Artificial Sequence
  Synthetic polypeptide
TPWWLRSPFFDY

SEQ ID NO: 24 - protein, 11 aa, Artificial Sequence
  Synthetic polypeptide
GGNNIGSTSVH

SEQ ID NO: 25 - protein, 7 aa, Artificial Sequence
  Synthetic polypeptide
DDSDRPS

SEQ ID NO: 26 - protein, 10 aa, Artificial Sequence
  Synthetic polypeptide
VRDSSSDHVI

SEQ ID NO: 27 - protein, 5 aa, Artificial Sequence
  Synthetic polypeptide
FSYWV

SEQ ID NO: 28 - protein, 16 aa, Artificial Sequence
  Synthetic polypeptide
TIYYSGNTYYNPSLKS

SEQ ID NO: 29 - protein, 11 aa, Artificial Sequence
  Synthetic polypeptide
RAGILTGYLDS

SEQ ID NO: 30 - protein, 11 aa, Artificial Sequence
  Synthetic polypeptide
GGNNIGSKSVH

SEQ ID NO: 31 - protein, 7 aa, Artificial Sequence
  Synthetic polypeptide
DDSDRPS

SEQ ID NO: 32 - protein, 11 aa, Artificial Sequence
  Synthetic polypeptide
QVWDSSSDHVV

SEQ ID NO: 33 - protein, 5 aa, Artificial Sequence
  Synthetic polypeptide
GCYMH

SEQ ID NO: 34 - protein, 17 aa, Artificial Sequence
  Synthetic polypeptide
RINPNSGGTNYAQKFQG

SEQ ID NO: 35 - protein, 9 aa, Artificial Sequence
  Synthetic polypeptide
DYSGWSFDY

SEQ ID NO: 36 - protein, 11 aa, Artificial Sequence
  Synthetic polypeptide
RASQGISNYLA

SEQ ID NO: 37 - protein, 7 aa, Artificial Sequence
  Synthetic polypeptide
AASSLQS

SEQ ID NO: 38 - protein, 10 aa, Artificial Sequence
  Synthetic polypeptide
QQYDSYPPTF

SEQ ID NO: 39 - protein, 5 aa, Artificial Sequence
  Synthetic polypeptide
SYYWG

SEQ ID NO: 40 - protein, 16 aa, Artificial Sequence
  Synthetic polypeptide
NIYYSGSTYYNPSLMS

SEQ ID NO: 41 - protein, 13 aa, Artificial Sequence
  Synthetic polypeptide
QLSYNWNDRLFDY

SEQ ID NO: 42 - protein, 11 aa, Artificial Sequence
  Synthetic polypeptide
GGNNIGSKNVH

SEQ ID NO: 43 - protein, 7 aa, Artificial Sequence
  Synthetic polypeptide
RDSNRPS

SEQ ID NO: 44 - protein, 9 aa, Artificial Sequence
  Synthetic polypeptide
QVWDSSTVV

SEQ ID NO: 45 - DNA, 1350 bp, Artificial Sequence
  Synthetic polynucleotide
cagctgctgctgcaagaatccggccctggcctggtgaaaccctccgagacactgtccctgacctgcaccgtgtccggcggctccctgtcctccagcttctcctactgggtctggatccggcagccccctggcaagggcctggaatggatcggcaccatctactactccggcaacacctactacaaccccagcctgaagtcccggctgaccatctccgtggacacctccaagaaccacttcagcctgaagctgtcctccgtgaccgccgctgacaccgccgtgtactactgtgccagaagggccggcatcctgaccggctacctggactcttggggccagggcaccctggtgacagtgtcctccgcctccaccaagggcccctccgtgttccctctggccccctccagcaagtccacctctggcggcaccgctgccctgggctgtctggtgaaagactacttccccgagcccgtgaccgtgtcctggaactctggcgccctgacctccggcgtgcacaccttccctgccgtgctgcagtcctccggcctgtactccctgtccagcgtggtgaccgtgccctccagctctctgggcacccagacctacatctgcaacgtgaaccacaagccctccaacaccaaggtggacaagcgggtggaacccaagtcctgcgacaagacccacacctgtcccccctgtcctgcccctgaagctgctggcggccctagcgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtctccaacaaggccctgcctgcccccatcgaaaagaccatctccaaggccaagggccagccccgcgagccccaggtgtacacactgccccctagccgggaagagatgaccaagaaccaggtgtccctgacatgcctggtgaagggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggc

SEQ ID NO: 46 - protein, 450 aa, Artificial Sequence
  Synthetic polypeptide
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - DNA, 642 bp, Artificial Sequence
  Synthetic polynucleotide
tcttacgtgctgacccagtccccctccgtgtccgtggctcctggccagaccgccagaatcacctgtggcggcaacaacatcggctccaagtccgtgcactggtatcagcagaagcccggccaggcccccgtgctggtggtgtacgacgactccgaccggccctctggcatccctgagcggttctccgcctccaactccggcaacaccgccaccctgaccatctccagagtggaagccggcgacgaggccgactactactgccaagtgtgggactcctcctccgaccacgtggtgttcggcggaggcaccaagctgaccgtgctgggccagcctaaggccgctccctccgtgaccctgttccccccatcctccgaggaactgcaggccaacaaggccaccctggtctgcctgatctccgacttctaccctggcgccgtgaccgtggcctggaaggccgacagctctcctgtgaaggccggcgtggaaaccaccaccccctccaagcagtccaacaacaaatacgccgcctcctcctacctgtccctgacccccgagcagtggaagtcccaccggtcctacagctgccaagtgacacacgagggctccaccgtggaaaagaccgtggcccctaccgagtgctcc

SEQ ID NO: 48 - protein, 214 aa, Artificial Sequence
  Synthetic polypeptide
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2023/0049011 A1
(43) Pub. Date: Feb. 16, 2023

(54) METHODS OF TREATING WARM AUTOIMMUNE HEMOLYTIC ANEMIA USING ANTI-FCRN ANTIBODIES

(71) Applicant: Immunovant Sciences GMBH, Basel (CH)

(72) Inventors: Christine Morel COQUERY, Durham, NC (US); Jonathon Andrew KLUFT, Tampa, FL (US); William Thomas SYMONDS, III, Cary, NC (US)

(21) Appl. No.: 17/777,913

(22) PCT Filed: Nov. 18, 2020
(86) PCT No.: PCT/US2020/061028

§ 371 Date: May 18, 2022

Related U.S. Application Data
(60) Provisional application No. 62/937,395, filed on Nov. 19, 2019.

(51) Int. Cl. C07K 16/28; A61P 37/00

(52) U.S. Cl. CPC C07K 16/283; A61P 37/00; C07K 2317/565; C07K 2317/92; A61K 2039/545

(57) ABSTRACT

The present disclosure relates to compositions, methods, and uses for using an isolated anti-FcRn antibody or an antigen-binding fragment thereof that binds to neonatal Fc receptor (FcRn) to prevent, modulate, or treat warm autoimmune hemolytic anemia.

The present disclosure claims the benefit of priority to U.S. Provisional Patent Application No. 62/937,395, filed Nov. 19, 2019, which is incorporated herein by reference in its entirety.

The instant application contains a Sequence Listing which has been submitted electronically in ASCII format and is incorporated herein by reference in its entirety. Said ASCII copy, created on Nov. 12, 2020, is named 15193_0005-00304_SL.txt and is 34,226 bytes in size.

The present disclosure relates to therapeutic methods, uses, and compositions comprising an isolated anti-FcRn antibody or an antigen-binding fragment thereof that binds to neonatal Fc receptor (FcRn) to prevent, modulate, or treat warm autoimmune hemolytic anemia. In certain aspects, the present disclosure provides methods of treating or preventing warm autoimmune hemolytic anemia by administering an anti-FcRn antibody or an antigen-binding fragment thereof to a patient in need thereof. In certain aspects, the present disclosure provides pharmaceutical compositions for treating or preventing warm autoimmune hemolytic anemia comprising an anti-FcRn antibody or an antigen-binding fragment thereof and at least one pharmaceutically acceptable carrier.

Antibodies are immunological proteins that bind to a specific antigen. In most animals, including humans and mice, antibodies are constructed from paired heavy and light polypeptide chains and each chain is made up of two distinct regions, referred to as the variable and constant regions. The heavy and light chain variable regions show significant sequence diversity between antibodies and are responsible for binding to the target antigen. The constant regions show less sequence diversity and are responsible for binding a number of natural proteins to elicit various biochemical events.

Under normal conditions, the average serum half-life of most IgG (i.e., IgG1, IgG2, and IgG4, excluding the IgG3 isotype) is about 21 days in humans (Morell et al., J. Clin. Invest. 49(4):673-80, 1970), which is a prolonged period relative to the serum half-life of other plasma proteins. With respect to this prolonged serum half-life of IgG, IgG that enters cells by endocytosis can strongly bind to neonatal Fc receptor (FcRn) in endosomes at a pH of 6.0 to avoid the degradative lysosomal pathway (FcRn, a type of Fc gamma receptor, is also referred to as FcRP, FcRB, or Brambell receptor). When the IgG-FcRn complex cycles to the plasma membrane, IgG dissociates rapidly from FcRn in the bloodstream at slightly basic pH (˜7.4). By this receptor-mediated recycling mechanism, FcRn effectively rescues the IgG from degradation in lysosomes, thereby prolonging the half-life of IgG (Roopenian et al., J. Immunol. 170:3528, 2003).

FcRn was identified in the neonatal rat gut, where it functions to mediate the absorption of IgG from the mother's milk and facilitate IgG transport to the circulatory system. FcRn has also been isolated from human placenta, where it mediates absorption and transport of maternal IgG to the fetal circulation. In adults, FcRn is expressed in a number of tissues, including epithelial tissues of the lung, intestine, kidney, as well as nasal, vaginal, and biliary tree surfaces.

FcRn is a non-covalent heterodimer that typically resides in the endosomes of endothelial and epithelial cells. FcRn is a membrane bound receptor having three heavy chain alpha domains (α1, α2, and α3) and a single soluble light chain β2-microglobulin (β2m) domain. Structurally, it belongs to a family of major histocompatibility complex class 1 molecules that have β2m as a common light chain. The FcRn chain has a molecular weight of about 46 kDa and is composed of an ectodomain containing the α1, α2, and α3 heavy chain domains and a β2m light chain domain and having a single sugar chain, a single-pass transmembrane, and a relatively short cytoplasmic tail.

In order to study the contributions of FcRn to IgG homeostasis, mice have been engineered to “knockout” at least part of the genes encoding β2m and FcRn heavy chains so that the proteins are not expressed. In these mice, the serum half-life and concentrations of IgG were dramatically reduced, suggesting an FcRn-dependent mechanism for IgG homeostasis. It has also been suggested that anti-human FcRn antibodies may be generated in these FcRn knockout mice, and that the antibodies may prevent binding of IgG to FcRn. The inhibition of IgG binding to FcRn negatively alters IgG serum half-life by preventing IgG recycling.

Autoimmune hemolytic anemia is a rare and heterogenous disease that affects approximately 1 to 3 per 100,000 patients per year (Michel, Expert Rev. Hematol. 4(6):607-18, 2011; Sokol et al., Br. Med. J. (Clin. Res. Ed.) 282(6281):2023-7, 1981). The pathology of the disease is likely caused by increased normal red blood cell (RBC) destruction triggered by autoantibodies reacting against RBC antigens with or without complement activation (Barcellini, Transfus. Med. Hemother. 42(5):287-93, 2015). Autoimmune hemolytic anemia is classified into three major types based on the optimal temperature in which the autoantibodies bind on the patient's RBCs in vivo: warm autoimmune hemolytic anemia, cold agglutinin syndrome, and paroxysmal cold hemoglobinuria. Warm autoimmune hemolytic anemia is the most common type of autoimmune hemolytic anemia, comprising ˜70% to ˜80% of all adult cases and ˜50% of the pediatric cases (Sokol et al., Br. Med. J. (Clin. Res. Ed.) 282(6281):2023-7, 1981).

In warm autoimmune hemolytic anemia, autoantibodies react optimally with the RBCs at about 37° C. RBCs coated by warm-reacting IgG are generally bound by spleen macrophages, which carry Fcγ receptors for the IgG heavy chain, and are either phagocytosed or form microsperocytes subject to further destruction during their next passage through the spleen (Kalfa, Hematology Am. Soc. Hematol. Educ. Program 2016(1):690-7, 2016). When either a high concentration of IgG or IgG with high affinity to complement is bound to RBCs, complement (C1q) can be bound and activated toward C3b. C3b-opsonized RBCs can then be phagocytosed by liver macrophages that carry C3b receptors, further contributing to the destruction of RBCs (Barcellini, Transfus. Med. Hemother. 42(5):287-93, 2015; Berentsen, Transfus. Med. Hemother. 42(5):303-10, 2015; LoBuglio et al., Science 158(3808):1582-5, 1967). Thus, autoantibodies such as IgG may play a role in the pathogenesis of warm autoimmune hemolytic anemia.

The present disclosure provides, in various embodiments, therapeutic methods, uses, and compositions for treating a patient suffering from warm autoimmune hemolytic anemia. The present disclosure more specifically provides, in various embodiments, a method of treating a patient suffering from warm autoimmune hemolytic anemia by administering to the patient a therapeutically effective amount of an anti-FcRn antibody or an antigen-binding fragment thereof. In various embodiments, the antibody or antigen-binding fragment is formulated as a pharmaceutical composition. Therapeutic uses of the antibodies, antigen-binding fragments, and pharmaceutical compositions described herein are also provided.

In various embodiments, treatment with an antibody, antigen-binding fragment, or pharmaceutical composition described herein reduces the level of at least one autoantibody and/or pathogenic antibody (e.g., at least one IgG, e.g., a pathogenic IgG (e.g., a pathogenic IgG1, IgG2, IgG3, or IgG4), serum IgG1, serum IgG2, serum IgG3, or serum IgG4) in a patient and/or in a sample from a patient, e.g., a patient suffering from warm autoimmune hemolytic anemia. In various embodiments, treatment with an antibody, antigen-binding fragment, or pharmaceutical composition described herein reduces the level of at least one autoantibody and/or pathogenic antibody (e.g., at least one IgG) in a patient and/or a sample from a patient by at least about 25%, about 35%, about 45%, about 50%, about 60%, about 70%, or about 80% relative to the level of the at least one autoantibody and/or pathogenic antibody in the patient and/or sample prior to treatment. In various embodiments, treatment with an antibody, antigen-binding fragment, or pharmaceutical composition described herein reduces the level of at least one IgG in a patient and/or a sample from a patient, e.g., a patient suffering from warm autoimmune hemolytic anemia. In various embodiments, the at least one IgG comprises a pathogenic IgG (e.g., a pathogenic IgG1, IgG2, IgG3, or IgG4). In various embodiments, the at least one IgG comprises serum IgG1. In various embodiments, the at least one IgG comprises serum IgG2. In various embodiments, the at least one IgG comprises serum IgG3. In various embodiments, the at least one IgG comprises serum IgG4.

In various embodiments, maximum reduction in the level of at least one autoantibody and/or pathogenic antibody (e.g., at least one IgG) occurs at about 5 days to about 30 days after administration of the antibody, antigen-binding fragment, or pharmaceutical composition. In some embodiments, maximum reduction in the level of at least one autoantibody and/or pathogenic antibody (e.g., at least one IgG) occurs at about 8 days following a single dose of the antibody, antigen-binding fragment, or pharmaceutical composition. In some embodiments, steady state is reached after about 3 to 4 doses of the antibody, antigen-binding fragment, or pharmaceutical composition.

In various embodiments, treatment with an antibody, antigen-binding fragment, or pharmaceutical composition described herein reduces the level of total serum IgG in a patient and/or in a sample from a patient, e.g., a patient suffering from warm autoimmune hemolytic anemia. In various embodiments, treatment with an antibody, antigen-binding fragment, or pharmaceutical composition described herein reduces the level of total serum IgG in a patient and/or a sample from a patient by at least about 25%, about 35%, about 45%, about 50%, about 60%, about 70%, or about 80% relative to the level of total serum IgG in the patient and/or sample prior to treatment. In various embodiments, treatment with an antibody, antigen-binding fragment, or pharmaceutical composition described herein reduces the level of total serum IgG in a patient and/or a sample from a patient by at least about 40% (e.g., about 40% to about 50%) after about 1 or 2 weeks of weekly dosing, relative to the level of total serum IgG in the patient and/or sample prior to treatment. In various embodiments, treatment with an antibody, antigen-binding fragment, or pharmaceutical composition described herein reduces the level of total serum IgG in a patient and/or a sample from a patient by at least about 60% (e.g., about 60% to about 70%) after about 3 weeks of weekly dosing, relative to the level of total serum IgG in the patient and/or sample prior to treatment. In various embodiments, treatment with an antibody, antigen-binding fragment, or pharmaceutical composition described herein reduces the level of total serum IgG in a patient and/or a sample from a patient by at least about 70% (e.g., about 70% to about 80%) after about 5 weeks of weekly dosing, relative to the level of total serum IgG in the patient and/or sample prior to treatment. In various embodiments, maximum reduction in the level of total serum IgG occurs at about 5 days to about 30 days after administration of the antibody, antigen-binding fragment, or pharmaceutical composition. In various embodiments, maximum reduction in the level of total serum IgG occurs after about 3 to 5 doses (e.g., after about 4 doses) of the antibody, antigen-binding fragment, or pharmaceutical composition.

In various embodiments, treatment with an antibody, antigen-binding fragment, or pharmaceutical composition described herein increases the level of hemoglobin in a patient and/or in a sample from a patient, e.g., a patient suffering from warm autoimmune hemolytic anemia. In various embodiments, treatment with an antibody, antigen-binding fragment, or pharmaceutical composition described herein increases the level of hemoglobin in a patient and/or a sample from a patient by at least about 5%, about 10%, about 15%, or about 20% (e.g., about 5% to about 30%) relative to the level of hemoglobin in the patient and/or sample prior to treatment. In various embodiments, treatment with an antibody, antigen-binding fragment, or pharmaceutical composition described herein increases the level of hemoglobin in a patient and/or a sample from a patient by at least about 10% (e.g., about 10% to about 15%) after about 1 or 2 weeks of weekly dosing, relative to the level of hemoglobin in the patient and/or sample prior to treatment. In various embodiments, treatment with an antibody, antigen-binding fragment, or pharmaceutical composition described herein increases the level of hemoglobin in a patient and/or a sample from a patient by at least about 20% (e.g., about 20% to about 25%) after about 1 or 2 weeks of weekly dosing, relative to the level of hemoglobin in the patient and/or sample prior to treatment. In some embodiments, an increase in the level of hemoglobin in a patient and/or a sample from a patient (e.g., an increase of about 10%, about 20%, or more) is maintained during the entire treatment period or a portion thereof. In some embodiments, an increase in the level of hemoglobin in a patient and/or a sample from a patient (e.g., an increase of about 10%, about 20%, or more) is maintained for at least 2, 3, or 4 weeks (e.g., 4 weeks or longer). In some embodiments, an increase in the level of hemoglobin in a patient and/or a sample from a patient (e.g., an increase of about 10%, about 20%, or more) is maintained for about 2 to about 6 weeks.

In various embodiments, the present disclosure provides therapeutic methods, uses, and compositions for treating or preventing warm autoimmune hemolytic anemia.

In various embodiments, the present disclosure provides a method of treating or preventing warm autoimmune hemolytic anemia in a patient in need thereof, comprising administering to the patient (i) a therapeutically effective amount of an anti-FcRn antibody or an antigen-binding fragment thereof; or (ii) a pharmaceutical composition comprising at least one pharmaceutically acceptable carrier and a therapeutically effective amount of an anti-FcRn antibody or an antigen-binding fragment thereof.

In various embodiments, the present disclosure provides an anti-FcRn antibody or an antigen-binding fragment thereof for use in a method of treating or preventing warm autoimmune hemolytic anemia in a patient in need thereof, the method comprising administering to the patient (i) a therapeutically effective amount of the antibody or antigen-binding fragment, or (ii) a pharmaceutical composition comprising at least one pharmaceutically acceptable carrier and a therapeutically effective amount of the antibody or antigen-binding fragment.

In various embodiments, the present disclosure provides a use of an anti-FcRn antibody or an antigen-binding fragment thereof in a method of treating or preventing warm autoimmune hemolytic anemia in a patient in need thereof, comprising administering to the patient (i) a therapeutically effective amount of the antibody or antigen-binding fragment; or (ii) a pharmaceutical composition comprising at least one pharmaceutically acceptable carrier and a therapeutically effective amount of the antibody or antigen-binding fragment.

In various embodiments, the present disclosure provides a use of an anti-FcRn antibody or an antigen-binding fragment thereof in the manufacture of a medicament for treating or preventing warm autoimmune hemolytic anemia in a patient in need thereof.

In various embodiments, the present disclosure provides a kit comprising an anti-FcRn antibody or an antigen-binding fragment thereof and instructions for use of the antibody or antigen-binding fragment in treating or preventing warm autoimmune hemolytic anemia in a patient in need thereof.

In various embodiments, the present disclosure provides a pharmaceutical composition for use in treating or preventing warm autoimmune hemolytic anemia in a patient in need thereof, the pharmaceutical composition comprising at least one pharmaceutically acceptable carrier and an anti-FcRn antibody or an antigen-binding fragment thereof.

In some embodiments of the therapeutic methods, uses, and compositions disclosed herein (e.g., for treating or preventing warm autoimmune hemolytic anemia), the antibody or antigen-binding fragment comprises a heavy chain variable region comprising an amino acid sequence of SEQ ID No: 27 (HCDR1), an amino acid sequence of SEQ ID No: 28 (HCDR2), and an amino acid sequence of SEQ ID No: 29 (HCDR3); and a light chain variable region comprising an amino acid sequence of SEQ ID No: 30 (LCDR1), an amino acid sequence of SEQ ID No: 31 (LCDR2), and an amino acid sequence of SEQ ID No: 32 (LCDR3). In some embodiments, the antibody or antigen-binding fragment comprises a heavy chain variable region comprising an amino acid sequence of SEQ ID No: 21 (HCDR1), an amino acid sequence of SEQ ID No: 22 (HCDR2), and an amino acid sequence of SEQ ID No: 23 (HCDR3); and a light chain variable region comprising an amino acid sequence of SEQ ID No: 24 (LCDR1), an amino acid sequence of SEQ ID No: 25 (LCDR2), and an amino acid sequence of SEQ ID No: 26 (LCDR3). In some embodiments, the therapeutically effective amount of the antibody or antigen-binding fragment is about 170 mg to about 1500 mg. In some embodiments, the therapeutically effective amount of the antibody or antigen-binding fragment is about 300 mg to about 800 mg. In some embodiments, the therapeutically effective amount of the antibody or antigen-binding fragment is about 340 mg or about 680 mg. In some embodiments, the therapeutically effective amount of the antibody or antigen-binding fragment is about 340 mg or about 680 mg administered once weekly. In some embodiments, the therapeutically effective amount of the antibody or antigen-binding fragment is about 340 mg or about 680 mg administered once weekly as one or more subcutaneous injections. In some embodiments, the therapeutically effective amount of the antibody or antigen-binding fragment is about 340 mg or about 680 mg (e.g., about 680 mg) administered once weekly for at least 2 weeks (e.g., 2 weeks, 3 weeks, 4 weeks, 5 weeks, 6 weeks, 7 weeks, 8 weeks, 10 weeks, 12 weeks, or longer). In some embodiments, the therapeutically effective amount of the antibody or antigen-binding fragment is about 340 mg or about 680 mg (e.g., about 680 mg) administered once weekly for at least 4 weeks. In some embodiments, the therapeutically effective amount of the antibody or antigen-binding fragment is about 340 mg or about 680 mg (e.g., about 680 mg) administered once weekly for at least 7 weeks. In some embodiments, the therapeutically effective amount of the antibody or antigen-binding fragment is about 340 mg or about 680 mg (e.g., about 680 mg) administered once weekly for at least 12 weeks.

In various embodiments of the therapeutic methods, uses, and compositions disclosed herein, the antibody or antigen-binding fragment is any of the antibodies or antigen-binding fragments disclosed in Intl. App. No. PCT/KR2015/004424 (Pub No. WO 2015/167293 A1), which is incorporated herein by reference.

In various embodiments of the therapeutic methods, uses, and compositions disclosed herein, the antibody or antigen-binding fragment comprises:

CDR1 comprising one or more amino acid sequences selected from the group consisting of SEQ ID Nos: 21, 24, 27, 30, 33, 36, 39, and 42;

CDR2 comprising one or more amino acid sequences selected from the group consisting of SEQ ID Nos: 22, 25, 28, 31, 34, 37, 40, and 43; and

CDR3 comprising one or more amino acid sequences selected from the group consisting of SEQ ID Nos: 23, 26, 29, 32, 35, 38, 41, and 44.

In various embodiments of the therapeutic methods, uses, and compositions disclosed herein, the antibody or antigen-binding fragment comprises:

CDR1 comprising an amino acid sequence that is at least 90% identical to one or more amino acid sequences selected from the group consisting of SEQ ID Nos: 21, 24, 27, 30, 33, 36, 39, and 42;

CDR2 comprising an amino acid sequence that is at least 90% identical to one or more amino acid sequences selected from the group consisting of SEQ ID Nos: 22, 25, 28, 31, 34, 37, 40, and 43; and

CDR3 comprising an amino acid sequence that is at least 90% identical to one or more amino acid sequences selected from the group consisting of SEQ ID Nos: 23, 26, 29, 32, 35, 38, 41, and 44.

In various embodiments, the antibody or antigen-binding fragment comprises a heavy chain variable region comprising an amino acid sequence of SEQ ID No: 27 (HCDR1), an amino acid sequence of SEQ ID No: 28 (HCDR2), and an amino acid sequence of SEQ ID No: 29 (HCDR3); and a light chain variable region comprising an amino acid sequence of SEQ ID No: 30 (LCDR1), an amino acid sequence of SEQ ID No: 31 (LCDR2), and an amino acid sequence of SEQ ID No: 32 (LCDR3). In various embodiments, the antibody or antigen-binding fragment comprises a heavy chain variable region comprising an amino acid sequence of SEQ ID No: 21 (HCDR1), an amino acid sequence of SEQ ID No: 22 (HCDR2), and an amino acid sequence of SEQ ID No: 23 (HCDR3); and a light chain variable region comprising an amino acid sequence of SEQ ID No: 24 (LCDR1), an amino acid sequence of SEQ ID No: 25 (LCDR2), and an amino acid sequence of SEQ ID No: 26 (LCDR3).

In various embodiments of the therapeutic methods, uses, and compositions disclosed herein, the antibody or antigen-binding fragment comprises one or more heavy chain variable regions and one or more light chain variable regions, wherein the heavy chain variable regions and light chain variable regions comprise one or more amino acid sequences selected from the group consisting of amino acid sequences of SEQ ID Nos: 2, 4, 6, 8, 10, 12, 14, 16, 18, and 20.

In various embodiments, the antibody or antigen-binding fragment comprises a heavy chain variable region comprising an amino acid sequence of SEQ ID No: 4 or SEQ ID No: 6; and a light chain variable region comprising an amino acid sequence of SEQ ID No: 14 or SEQ ID No: 16. In various embodiments, the antibody or antigen-binding fragment comprises a heavy chain variable region comprising an amino acid sequence of SEQ ID No: 6; and a light chain variable region comprising an amino acid sequence of SEQ ID No: 16. In various embodiments, the antibody or antigen-binding fragment comprises a heavy chain variable region comprising an amino acid sequence of SEQ ID No: 4; and a light chain variable region comprising an amino acid sequence of SEQ ID No: 14. In various embodiments, the antibody or antigen-binding fragment comprises a heavy chain variable region comprising an amino acid sequence of SEQ ID No: 2; and a light chain variable region comprising an amino acid sequence of SEQ ID No: 12.

In various embodiments of the therapeutic methods, uses, and compositions disclosed herein, the antibody or antigen-binding fragment comprises one or more heavy chain variable regions and one or more light chain variable regions, wherein the heavy chain variable regions and light chain variable regions comprise an amino acid sequence that is at least 90% identical to one or more amino acid sequences selected from the group consisting of amino acid sequences of SEQ ID Nos: 2, 4, 6, 8, 10, 12, 14, 16, 18, and 20. In various embodiments, the heavy chain variable regions and light chain variable regions comprise an amino acid sequence that is at least 91%, at least 92%, at least 93%, at least 94%, at least 95%, at least 96%, at least 97%, at least 98%, or at least 99% identical to one or more amino acid sequences selected from the group consisting of amino acid sequences of SEQ ID Nos: 2, 4, 6, 8, 10, 12, 14, 16, 18, and 20.

In various embodiments, the antibody or antigen-binding fragment comprises a heavy chain variable region comprising an amino acid sequence that is at least 90% identical to SEQ ID No: 6; and a light chain variable region comprising an amino acid sequence that is at least 90% identical to SEQ ID No: 16. In various embodiments, the antibody or antigen-binding fragment comprises a heavy chain variable region comprising an amino acid sequence that is at least 90% identical to SEQ ID No: 4; and a light chain variable region comprising an amino acid sequence that is at least 90% identical to SEQ ID No: 14. In various embodiments, the antibody or antigen-binding fragment comprises a heavy chain variable region comprising an amino acid sequence that is at least 90% identical to SEQ ID No: 2; and a light chain variable region comprising an amino acid sequence that is at least 90% identical to SEQ ID No: 12.

In various embodiments, the antibody or antigen-binding fragment binds to FcRn with a K_{D} (dissociation constant) of about 0.01 nM to about 2 nM at pH 6.0 or pH 7.4, as measured by, e.g., surface plasmon resonance (SPR). In various embodiments, the K_{D} is measured by surface plasmon resonance (e.g., human FcRn-immobilized surface plasmon resonance). In various embodiments, the K_{D} is measured by human FcRn-immobilized surface plasmon resonance.

In various embodiments, the antibody or antigen-binding fragment is any of the antibodies or antigen-binding fragments disclosed or incorporated by reference herein.

In various embodiments of the therapeutic methods, uses, and compositions disclosed herein, a patient or a sample from a patient (e.g., a patient affected with warm autoimmune hemolytic anemia) has a detectable level of an anti-red blood cell IgG (anti-RBC IgG). In some embodiments, the anti-RBC IgG is an anti-RBC IgG1. In some embodiments, the anti-RBC IgG is an anti-RBC IgG2. In some embodiments, the anti-RBC IgG is an anti-RBC IgG3. In some embodiments, the anti-RBC IgG is an anti-RBC IgG4.

In various embodiments of the therapeutic methods, uses, and compositions disclosed herein, the antibody, antigen-binding fragment, or pharmaceutical composition is administered subcutaneously. In various embodiments, the antibody, antigen-binding fragment, or pharmaceutical composition is administered as one or more subcutaneous injections. In various embodiments, the antibody, antigen-binding fragment, or pharmaceutical composition is administered as one or more intravenous injections. In various embodiments, the antibody, antigen-binding fragment, or pharmaceutical composition is administered without intravenous administration (e.g., intravenous induction) prior to one or more subcutaneous injections. In various embodiments, the antibody, antigen-binding fragment, or pharmaceutical composition is contained in a syringe prior to administration. In various embodiments, the antibody, antigen-binding fragment, or pharmaceutical composition is administered as a single (i.e., one) subcutaneous injection. In various embodiments, the antibody, antigen-binding fragment, or pharmaceutical composition is administered as two or more (e.g., two) consecutive subcutaneous injections. In various embodiments, the antibody, antigen-binding fragment, or pharmaceutical composition is administered as a fixed dose.

In some embodiments, the antibody, antigen-binding fragment, or pharmaceutical composition is administered to the patient once as a single dose or once weekly. In some embodiments, the antibody, antigen-binding fragment, or pharmaceutical composition is administered to the patient once weekly as a single subcutaneous injection. In some embodiments, the antibody, antigen-binding fragment, or pharmaceutical composition is administered to the patient once weekly as two or more consecutive subcutaneous injections. In some embodiments, the antibody, antigen-binding fragment, or pharmaceutical composition is administered to the patient once weekly for at least 2 weeks, at least 3 weeks, at least 4 weeks, at least 5 weeks, at least 6 weeks, at least 7 weeks, at least 8 weeks, at least 9 weeks, at least 10 weeks, at least 12 weeks, at least 20 weeks, at least 24 weeks, at least 30 weeks, at least 40 weeks, at least 50 weeks, at least 60 weeks, at least 70 weeks, at least 76 weeks, at least 80 weeks, or longer. In some embodiments, the antibody, antigen-binding fragment, or pharmaceutical composition is administered to the patient once weekly for at least 4 weeks. In some embodiments, the antibody, antigen-binding fragment, or pharmaceutical composition is administered to the patient once weekly for 6 to 76 weeks, or any time period in between. In some embodiments, the antibody, antigen-binding fragment, or pharmaceutical composition is administered to the patient once weekly for at least 6 weeks. In some embodiments, the antibody, antigen-binding fragment, or pharmaceutical composition is administered to the patient once weekly for at least 7 weeks. In some embodiments, the antibody, antigen-binding fragment, or pharmaceutical composition is administered to the patient once weekly for at least 12 weeks. In some embodiments, the antibody, antigen-binding fragment, or pharmaceutical composition is administered to the patient once weekly for at least 24 weeks. In some embodiments, the antibody, antigen-binding fragment, or pharmaceutical composition is administered to the patient once weekly for at least 76 weeks. In some embodiments, the antibody, antigen-binding fragment, or pharmaceutical composition is administered to the patient once weekly until sufficient to treat, prevent, reduce the severity, delay the onset, and/or reduce the risk of occurrence of one or more symptoms of warm autoimmune hemolytic anemia.

In some embodiments, the patient has warm autoimmune hemolytic anemia. In some embodiments, the antibody, antigen-binding fragment, or pharmaceutical composition is administered to the patient once weekly as a single subcutaneous injection for at least 4 weeks (e.g., at a dose of about 340 mg). In some embodiments, the antibody, antigen-binding fragment, or pharmaceutical composition is administered to the patient once weekly as a single subcutaneous injection for at least 7 weeks (e.g., at a dose of about 340 mg). In some embodiments, the antibody, antigen-binding fragment, or pharmaceutical composition is administered to the patient once weekly as a single subcutaneous injection for at least 12 weeks (e.g., at a dose of about 340 mg). In some embodiments, the antibody, antigen-binding fragment, or pharmaceutical composition is administered to the patient once weekly as two or more (e.g., two) consecutive subcutaneous injections for at least 4 weeks (e.g., at a dose of about 680 mg). In some embodiments, the antibody, antigen-binding fragment, or pharmaceutical composition is administered to the patient once weekly as two or more (e.g., two) consecutive subcutaneous injections for at least 7 weeks (e.g., at a dose of about 680 mg). In some embodiments, the antibody, antigen-binding fragment, or pharmaceutical composition is administered to the patient once weekly as two or more (e.g., two) consecutive subcutaneous injections for at least 12 weeks (e.g., at a dose of about 680 mg). In some embodiments, the antibody, antigen-binding fragment, or pharmaceutical composition is administered to the patient once weekly as one or more subcutaneous injections until sufficient to treat, prevent, reduce the severity, delay the onset, and/or reduce the risk of occurrence of one or more symptoms of the patient's warm autoimmune hemolytic anemia. In some embodiments, the antibody, antigen-binding fragment, or pharmaceutical composition is administered to the patient at a dose of about 340 mg or about 680 mg.

In some embodiments, the antibody, antigen-binding fragment, or pharmaceutical composition is administered to the patient once every 2 weeks (bi-weekly). In some embodiments, the antibody, antigen-binding fragment, or pharmaceutical composition is administered to the patient once every 2 weeks as a single subcutaneous injection. In some embodiments, the antibody, antigen-binding fragment, or pharmaceutical composition is administered to the patient once every 2 weeks as two or more consecutive subcutaneous injections. In some embodiments, the antibody, antigen-binding fragment, or pharmaceutical composition is administered to the patient once every 2 weeks for at least 2 weeks, at least 4 weeks, at least 6 weeks, at least 8 weeks, at least 10 weeks, at least 12 weeks, at least 20 weeks, at least 24 weeks, at least 30 weeks, at least 40 weeks, at least 50 weeks, at least 60 weeks, at least 70 weeks, at least 76 weeks, at least 80 weeks, or longer. In some embodiments, the antibody, antigen-binding fragment, or pharmaceutical composition is administered to the patient once every 2 weeks for 6 to 76 weeks, or any time period in between. In some embodiments, the antibody, antigen-binding fragment, or pharmaceutical composition is administered to the patient once every 2 weeks for at least 6 weeks. In some embodiments, the antibody, antigen-binding fragment, or pharmaceutical composition is administered to the patient once every 2 weeks for at least 12 weeks. In some embodiments, the antibody, antigen-binding fragment, or pharmaceutical composition is administered to the patient once every 2 weeks for at least 24 weeks. In some embodiments, the antibody, antigen-binding fragment, or pharmaceutical composition is administered to the patient once every 2 weeks for at least 76 weeks. In some embodiments, the antibody, antigen-binding fragment, or pharmaceutical composition is administered to the patient once every 2 weeks until sufficient to treat, prevent, reduce the severity, delay the onset, and/or reduce the risk of occurrence of one or more symptoms of warm autoimmune hemolytic anemia.

In some embodiments, the antibody, antigen-binding fragment, or pharmaceutical composition is administered to the patient once monthly. In some embodiments, the antibody, antigen-binding fragment, or pharmaceutical composition is administered to the patient once monthly as a single subcutaneous injection. In some embodiments, the antibody, antigen-binding fragment, or pharmaceutical composition is administered to the patient once monthly as two or more consecutive subcutaneous injections. In some embodiments, the antibody, antigen-binding fragment, or pharmaceutical composition is administered to the patient once monthly for at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or longer. In some embodiments, the antibody, antigen-binding fragment, or pharmaceutical composition is administered to the patient once monthly until sufficient to treat, prevent, reduce the severity, delay the onset, and/or reduce the risk of occurrence of one or more symptoms of warm autoimmune hemolytic anemia.

In some embodiments, the antibody, antigen-binding fragment, or pharmaceutical composition is administered to the patient once or more than once over a period of about 1 day, 2 days, 3 days, 4 days, 5 days, 6 days, 7 days, 8 days, 9 days, 10 days, 11 days, 12 days, 13 days, 14 days, 1 week, 2 weeks, 3 weeks, 4 weeks, 1 month, 2 months, 3 months, 4 months, 5 months, 6 months, 7 months, 8 months, 9 months, 10 months, 11 months, 12 months, 18 months, 24 months, 30 months, 36 months, or longer.

In various embodiments, the antibody, antigen-binding fragment, or pharmaceutical composition is self-administered by the patient. In various embodiments, the antibody, antigen-binding fragment, or pharmaceutical composition is self-administered by the patient at home. In various embodiments, the antibody, antigen-binding fragment, or pharmaceutical composition is administered by a treating clinician. In various embodiments, the antibody, antigen-binding fragment, or pharmaceutical composition is administered alone, i.e., as a single agent. In various embodiments, the antibody, antigen-binding fragment, or pharmaceutical composition is administered in combination with at least one additional therapeutic agent.

In various embodiments of the therapeutic methods, uses, and compositions disclosed herein, the therapeutically effective amount of the antibody or antigen-binding fragment is about 170 mg to about 300 mg. In various embodiments, the therapeutically effective amount of the antibody or antigen-binding fragment is about 300 mg to about 500 mg. In various embodiments, the therapeutically effective amount of the antibody or antigen-binding fragment is about 500 mg to about 700 mg. In various embodiments, the therapeutically effective amount of the antibody or antigen-binding fragment is about 700 mg to about 900 mg. In various embodiments, the therapeutically effective amount of the antibody or antigen-binding fragment is about 900 mg to about 1100 mg. In various embodiments, the therapeutically effective amount of the antibody or antigen-binding fragment is about 1100 mg to about 1300 mg. In various embodiments, the therapeutically effective amount of the antibody or antigen-binding fragment is about 1300 mg to about 1500 mg. In various embodiments, the therapeutically effective amount of the antibody or antigen-binding fragment is the amount required to reduce the level of at least one autoantibody and/or pathogenic antibody (e.g., at least one IgG) in a patient and/or a sample from a patient by at least about 25%, about 35%, about 45%, about 50%, about 60%, about 70%, about 80%, or more. In various embodiments, the therapeutically effective amount of the antibody or antigen-binding fragment is the amount required to reduce the level of total serum IgG in a patient and/or a sample from a patient by at least about 25%, about 35%, about 45%, about 50%, about 60%, about 70%, about 80%, or more. In various embodiments, the therapeutically effective amount of the antibody or antigen-binding fragment is the amount required to increase the level of hemoglobin in a patient and/or a sample from a patient by about 5%, about 10%, about 15%, about 20%, or more.

In various embodiments of the therapeutic methods, uses, and compositions disclosed herein, the therapeutically effective amount of the antibody or antigen-binding fragment is about 300 mg to about 900 mg. In various embodiments, the therapeutically effective amount of the antibody or antigen-binding fragment is about 300 mg to about 900 mg administered once weekly or once every 2 weeks. In various embodiments, the therapeutically effective amount of the antibody or antigen-binding fragment is about 300 mg to about 400 mg, about 400 mg to about 500 mg, about 500 mg to about 600 mg, about 600 mg to about 700 mg, about 700 mg to about 800 mg, or about 800 mg to about 900 mg. In various embodiments, the therapeutically effective amount of the antibody or antigen-binding fragment is about 300 mg to about 400 mg, about 400 mg to about 500 mg, about 500 mg to about 600 mg, about 600 mg to about 700 mg, about 700 mg to about 800 mg, or about 800 mg to about 900 mg administered once weekly or once every 2 weeks.

In various embodiments, the therapeutically effective amount of the antibody or antigen-binding fragment is about 300 mg to about 400 mg (e.g., about 300 mg to about 350 mg, e.g., about 340 mg). In various embodiments, the therapeutically effective amount of the antibody or antigen-binding fragment is about 340 mg. In various embodiments, the therapeutically effective amount of the antibody or antigen-binding fragment is about 340 mg administered once weekly. In various embodiments, the therapeutically effective amount of the antibody or antigen-binding fragment is about 340 mg administered once weekly as a single subcutaneous injection. In some embodiments, the therapeutically effective amount of the antibody or antigen-binding fragment is about 340 mg administered once weekly for at least 2 weeks (e.g., 2 weeks, 3 weeks, 4 weeks, 5 weeks, 6 weeks, 7 weeks, 8 weeks, 10 weeks, 12 weeks, or longer). In some embodiments, the therapeutically effective amount of the antibody or antigen-binding fragment is about 340 mg administered once weekly for at least 4 weeks. In some embodiments, the therapeutically effective amount of the antibody or antigen-binding fragment is about 340 mg administered once weekly for at least 7 weeks. In some embodiments, the therapeutically effective amount of the antibody or antigen-binding fragment is about 340 mg administered once weekly for at least 12 weeks.

In various embodiments, the therapeutically effective amount of the antibody or antigen-binding fragment is about 650 mg to about 750 mg (e.g., about 650 mg to about 700 mg, e.g., about 680 mg). In various embodiments, the therapeutically effective amount of the antibody or antigen-binding fragment is about 680 mg. In various embodiments, the therapeutically effective amount of the antibody or antigen-binding fragment is about 680 mg administered once weekly. In various embodiments, the therapeutically effective amount of the antibody or antigen-binding fragment is about 680 mg administered once weekly as two or more (e.g., two) consecutive subcutaneous injections. In various embodiments, each subcutaneous injection comprises an approximately equal amount (e.g., about 340 mg) of the antibody or antigen-binding fragment. In some embodiments, the therapeutically effective amount of the antibody or antigen-binding fragment is about 680 mg administered once weekly for at least 2 weeks (e.g., 2 weeks, 3 weeks, 4 weeks, 5 weeks, 6 weeks, 7 weeks, 8 weeks, 10 weeks, 12 weeks, or longer). In some embodiments, the therapeutically effective amount of the antibody or antigen-binding fragment is about 680 mg administered once weekly for at least 4 weeks. In some embodiments, the therapeutically effective amount of the antibody or antigen-binding fragment is about 680 mg administered once weekly for at least 7 weeks. In some embodiments, the therapeutically effective amount of the antibody or antigen-binding fragment is about 680 mg administered once weekly for at least 12 weeks.

In various embodiments of the therapeutic methods, uses, and compositions disclosed herein, treatment with an antibody, antigen-binding fragment, or pharmaceutical composition of the present disclosure reduces the level of at least one autoantibody and/or pathogenic antibody (e.g., at least one IgG) in a patient and/or a sample from a patient, e.g., a patient suffering from warm autoimmune hemolytic anemia. In some embodiments, treatment reduces the level of at least one autoantibody and/or pathogenic antibody (e.g., at least one IgG) in a patient and/or a sample from a patient by at least about 20%, about 25%, about 30%, about 35%, about 40%, about 45%, about 50%, about 55%, about 60%, about 65%, about 70%, about 75%, about 80%, about 85%, or about 90%, i.e., relative to the level of the at least one autoantibody and/or pathogenic antibody in the patient and/or sample prior to treatment. In some embodiments, treatment reduces the level of at least one autoantibody and/or pathogenic antibody (e.g., at least one IgG) in a patient and/or a sample from a patient by at least about 40%, about 45%, about 50%, about 55%, about 60%, about 65%, about 70%, about 75%, about 80%, about 85%, or about 90%, i.e., relative to the level of the at least one autoantibody and/or pathogenic antibody in the patient and/or sample prior to treatment. In some embodiments, the level of at least one autoantibody and/or pathogenic antibody (e.g., at least one IgG) is measured at the start of treatment and/or at about 1 week, about 2 weeks, about 3 weeks, about 4 weeks, about 5 weeks, about 6 weeks, about 7 weeks, and/or about 8 weeks following the start of treatment. In some embodiments, maximum reduction in the level of the at least one autoantibody and/or pathogenic antibody (e.g., at least one IgG) in the patient occurs at about 5 days to about 40 days or about 5 days to about 30 days after administration of the antibody, antigen-binding fragment, or pharmaceutical composition. In some embodiments, maximum reduction in the level of the at least one autoantibody and/or pathogenic antibody (e.g., at least one IgG) in the patient occurs at about 15 days to about 30 days after administration of the antibody, antigen-binding fragment, or pharmaceutical composition.

In some embodiments, the at least one IgG comprises a pathogenic IgG (e.g., a pathogenic IgG1, IgG2, IgG3, or IgG4). In some embodiments, the at least one IgG comprises an anti-RBC IgG (e.g., an anti-RBC IgG1, an anti-RBC IgG2, an anti-RBC IgG3, and/or an anti-RBC IgG4). In some embodiments, the at least one IgG comprises an IgG1, an IgG2, an IgG3, or an IgG4. In some embodiments, the at least one IgG comprises serum IgG1. In some embodiments, the at least one IgG comprises serum IgG2. In some embodiments, the at least one IgG comprises serum IgG3. In some embodiments, the at least one IgG comprises serum IgG4.

In various embodiments of the therapeutic methods, uses, and compositions disclosed herein, treatment with an anti-FcRn antibody, antigen-binding fragment, or pharmaceutical composition of the present disclosure reduces the level of total serum IgG in a patient and/or a sample from a patient, e.g., a patient suffering from warm autoimmune hemolytic anemia. In some embodiments, treatment reduces the level of total serum IgG in a patient and/or a sample from a patient by at least about 20%, about 25%, about 30%, about 35%, about 40%, about 45%, about 50%, about 55%, about 60%, about 65%, about 70%, about 75%, about 80%, about 85%, or about 90%, i.e., relative to the level of total serum IgG in the patient and/or sample prior to treatment. In some embodiments, treatment reduces the level of total serum IgG in a patient and/or a sample from a patient by at least about 40%, about 45%, about 50%, about 55%, about 60%, about 65%, about 70%, about 75%, about 80%, about 85%, or about 90%, i.e., relative to the level of total serum IgG in the patient and/or sample prior to treatment. In various embodiments, treatment reduces the level of total serum IgG in a patient and/or a sample from a patient by at least about 40% (e.g., about 40% to about 50%) after about 1 or 2 weeks of weekly dosing, i.e., relative to the level of total serum IgG in the patient and/or sample prior to treatment. In various embodiments, treatment reduces the level of total serum IgG in a patient and/or a sample from a patient by at least about 60% (e.g., about 60% to about 70%) after about 3 weeks of weekly dosing, i.e., relative to the level of total serum IgG in the patient and/or sample prior to treatment. In various embodiments, treatment reduces the level of total serum IgG in a patient and/or a sample from a patient by at least about 70% (e.g., about 70% to about 80%) after about 5 weeks of weekly dosing, i.e., relative to the level of total serum IgG in the patient and/or sample prior to treatment. In some embodiments, the level of total serum IgG is measured at the start of treatment and/or at about 1 week, about 2 weeks, about 3 weeks, about 4 weeks, about 5 weeks, about 6 weeks, about 7 weeks, and/or about 8 weeks following the start of treatment. In some embodiments, maximum reduction in the level of total serum IgG in the patient occurs at about 5 days to about 40 days or about 5 days to about 30 days after administration of the antibody, antigen-binding fragment, or pharmaceutical composition. In some embodiments, maximum reduction in the level of total serum IgG in the patient occurs at about 15 days to about 30 days after administration of the antibody, antigen-binding fragment, or pharmaceutical composition. In some embodiments, maximum reduction in the level of total serum IgG occurs after about 3 to 5 doses (e.g., after about 4 doses) of the antibody, antigen-binding fragment, or pharmaceutical composition.

In various embodiments of the therapeutic methods, uses, and compositions disclosed herein, treatment with an anti-FcRn antibody, antigen-binding fragment, or pharmaceutical composition of the present disclosure increases the level of hemoglobin in a patient and/or a sample from a patient, e.g., a patient suffering from warm autoimmune hemolytic anemia. In various embodiments, treatment increases the level of hemoglobin in a patient and/or a sample from a patient by at least about 5%, about 10%, about 15%, or about 20% (e.g., about 5% to about 30%), i.e., relative to the level of hemoglobin in the patient and/or sample prior to treatment. In various embodiments, treatment increases the level of hemoglobin in a patient and/or a sample from a patient by at least about 10% (e.g., about 10% to about 15%) after about 1 or 2 weeks of weekly dosing, i.e., relative to the level of hemoglobin in the patient and/or sample prior to treatment. In various embodiments, treatment increases the level of hemoglobin in a patient and/or a sample from a patient by at least about 20% (e.g., about 20% to about 25%) after about 1 or 2 weeks of weekly dosing, i.e., relative to the level of hemoglobin in the patient and/or sample prior to treatment. In some embodiments, an increase in the level of hemoglobin in a patient and/or a sample from a patient (e.g., an increase of about 10%, about 20%, or more) is maintained during the entire treatment period or a portion thereof. In some embodiments, an increase in the level of hemoglobin in a patient and/or a sample from a patient (e.g., an increase of about 10%, about 20%, or more) is maintained for at least 2, 3, or 4 weeks (e.g., 4 weeks or longer). In some embodiments, an increase in the level of hemoglobin in a patient and/or a sample from a patient (e.g., an increase of about 10%, about 20%, or more) is maintained for about 2 to about 6 weeks.

### BRIEF DESCRIPTION OF THE DRAWINGS

FIG. 1 shows the results of analyzing the expression of antibodies in CHO-S cells and analyzing HL161A, HL161B, HL161C, and HL161D antibody proteins, obtained by protein A purification, on SDS-PAGE gel under a reduced or non-reduced condition. Under a non-reduced condition, each of the HL161 antibodies had a whole human IgG1 type structure having a size of about 160 kDa, and under a reduced condition, the heavy chain had a size of about 55 kDa, and the light chain had a size of about 25 kDa. In FIG. 1, lane 1 represents a molecular weight (M.W.) marker, lane 2 represents 2 μg non-reduced (*NEM-treated) antibody, and lane 3 represents 2 μg reduced antibody.

FIG. 2A to FIG. 2H show the results of analysis performed using a surface plasmon resonance (SPR) system in order to determine the kinetic dissociation (K_{D}) of four antibodies (HL161A, HL161B, HL161C, and HL161D) that bind to FcRn. The results in FIG. 2A to FIG. 2H were obtained by analyzing the interaction between human FcRn and the HL161A, HL161B, HL161C, or HL161D antibody at pH 6.0 and pH 7.4 using a Proteon GLC chip and a Proteon XPR36 (Bio-Rad) system.

FIG. 2A shows the results of analyzing the interaction between human FcRn and the HL161A antibody at pH 6.0.

FIG. 2B shows the results of analyzing the interaction between human FcRn and the HL161A antibody at pH 7.4.

FIG. 2C shows the results of analyzing the interaction between human FcRn and the HL161B antibody at pH 6.0.

FIG. 2D shows the results of analyzing the interaction between human FcRn and the HL161B antibody at pH 7.4.

FIG. 2E shows the results of analyzing the interaction between human FcRn and the HL161C antibody at pH 6.0.

FIG. 2F shows the results of analyzing the interaction between human FcRn and the HL161C antibody at pH 7.4.

FIG. 2G shows the results of analyzing the interaction between human FcRn and the HL161D antibody at pH 6.0.

FIG. 2H shows the results of analyzing the interaction between human FcRn and the HL161D antibody at pH 7.4.

FIG. 3 shows the ability of two selected antibodies to bind to the cell surface, and shows the results obtained by treating human FcRn-overexpressing HEK293 cells with selected HL161A and HL161B antibodies binding to human FcRn present on the cell surface and analyzing the antibodies binding to the cell surface at pH 6.0 and pH 7.4. The binding of each of the HL161A and HL161B antibodies to human FcRn was expressed as an MFI value obtained by performing fluorescent activated cell sorter (FACS) using Alexa488-labeled anti-human goat antibody after treating cells with each antibody at varying pHs.

FIG. 4 shows the results of analyzing the ability to block the binding of human IgG to human FcRn-expressing cells at pH 6.0, and shows the results of observing whether two selected antibodies binding to cell surface human FcRn can block the binding of human IgG to human FcRn, at the cell level. A profile of the ability to block the binding of Alexa488-labeled human IgG to human FcRn was obtained by diluting each of HL161A and HL161B antibodies, confirmed to bind to human FcRn-overexpressing HEK293 cells, serially 4-fold from 200 nM.

FIG. 5A and FIG. 5B show the results of analyzing the effects of HL161A and HL161B antibodies, selected from human FcRn-expressing transgenic mouse Tg32 (hFcRn+/+, hβ2m+/+, mFcRn−/−, mβ2m−/−), on the catabolism of hIgG1. At 0 hour, 5 mg/kg of biotin-hIgG and 495 mg/kg of human IgG were intraperitoneally administered to saturate IgG in vivo. Regarding drug administration, at 24, 48, 72, and 96 hours after administration of biotin-IgG, IgG1, HL161A, HL161B, or PBS was injected intraperitoneally at doses of 5, 10 and 20 mg/kg once a day. Sample collection was performed at 24, 48, 72, 96, 120, and 168 hours after administration of biotin-IgG. At 24, 48, 72, and 96 hours, blood was collected before drug administration, and the remaining amount of biotin-IgG was analyzed by an ELISA method. The results were expressed as the ratio of the remaining amount at each time point to 100% for the remaining amount in the blood sample collected at 24 hours.

FIG. 6A to FIG. 6C show the results of analyzing the change in blood level of monkey IgG caused by administration of two antibodies (HL161A and HL161B) to cynomolgus monkeys having a sequence homology of 96% to human FcRn. Each of HL161A and HL161B antibodies was administered intravenously to cynomolgus monkeys at doses of 5 mg/kg and 20 mg/kg once a day.

FIG. 6A shows the serum IgG-reducing effects of HL161A and HL161B antibodies at varying antibody concentrations.

FIG. 6B shows the serum IgG-reducing effects of HL161A and HL161B antibodies (concentration: (5 mg/kg) in monkey individuals).

FIG. 6C shows the serum IgG-reducing effects of HL161A and HL161B antibodies (concentration: (20 mg/kg) in monkey individuals).

FIG. 7A and FIG. 7B show the results of analyzing the pharmacokinetic profiles of HL161A and HL161B in an experiment performed using cynomolgus monkeys.

FIG. 8A to FIG. 8C show the results of analyzing the changes in blood levels of monkey IgM, IgA, and albumin caused by administration of HL161A and HL161B antibodies in an experiment performed using cynomolgus monkeys.

FIG. 8A shows a change in the serum IgM level of monkeys.

FIG. 8B shows a change in the serum IgA level of monkeys.

FIG. 8C shows a change in the serum albumin level of monkeys.

FIG. 9 shows single and multiple doses of RVT-1401 (HL161BKN) in healthy subjects following subcutaneous (SC) or intravenous (IV) administration (N=RVT-1401:placebo).

FIG. 10A and FIG. 10B show mean concentration-time profiles in healthy subjects following single dose IV and SC administration of RVT-1401 (FIG. 10A: IV; FIG. 10B: SC).

FIG. 11A and FIG. 11B show mean concentration-time profiles in healthy subjects following weekly SC administration of RVT-1401 at 340 mg or 680 mg (FIG. 11A: linear plot; FIG. 11B: semi-log plot).

FIG. 12 shows serum IgG concentration-time profiles in healthy subjects following weekly SC administration of RVT-1401 at 340 mg or 680 mg.

FIG. 13A shows percent (%) serum IgG reduction from baseline in healthy subjects following single dose IV administration of RVT-1401 (340 mg, 765 mg, 1530 mg) or placebo. Arrow indicates time of RVT-1401 administration. FIG. 13B shows percent (%) serum IgG reduction from baseline in healthy subjects following single dose SC administration of RVT-1401 (340 mg, 765 mg) or placebo. Arrow indicates time of RVT-1401 administration.

FIG. 14A to FIG. 14E show percent (%) serum IgG (total and subclass) reduction from baseline in healthy subjects following multiple dose SC administration of RVT-1401 (340 mg, 680 mg) or placebo. Arrows indicate time of RVT-1401 administration (once weekly×4 weeks).

FIG. 14A shows percent (%) serum IgG (total) reduction from baseline in healthy subjects following multiple dose SC administration of RVT-1401 (340 mg, 680 mg) or placebo.

FIG. 14B shows percent (%) serum IgG1 reduction from baseline in healthy subjects following multiple dose SC administration of RVT-1401 (340 mg, 680 mg) or placebo.

FIG. 14C shows percent (%) serum IgG2 reduction from baseline in healthy subjects following multiple dose SC administration of RVT-1401 (340 mg, 680 mg) or placebo.

FIG. 14D shows percent (%) serum IgG3 reduction from baseline in healthy subjects following multiple dose SC administration of RVT-1401 (340 mg, 680 mg) or placebo.

FIG. 14E shows percent (%) serum IgG4 reduction from baseline in healthy subjects following multiple dose SC administration of RVT-1401 (340 mg, 680 mg) or placebo.

FIG. 15 shows the study design of a non-randomized, open-label study to assess the safety, tolerability, PK, PD, and efficacy of RVT-1401 (680 mg weekly and 340 mg weekly) in patients with warm autoimmune hemolytic anemia (WAIHA). Patients diagnosed with WAIHA are treated with once weekly SC injections of RVT-1401: Dosing Regimen A (680 mg weekly for 12 weeks (Cohort 1)), and Dosing Regimen B (340 mg weekly for 12 weeks (Cohort 2)). Dosing Regimen A (680 mg weekly) is administered as two SC injections per week, and Dosing Regimen B (340 mg weekly) is administered as a single SC injection per week. Asterisks (**) indicate that Cohort 1 enrolls first, followed by Cohort 2.

### DETAILED DESCRIPTION

In order that the disclosure may be more readily understood, certain terms are defined throughout the detailed description. Unless defined otherwise herein, all scientific and technical terms used in connection with the present disclosure have the same meaning as commonly understood by those of ordinary skill in the art. All references cited herein are also incorporated by reference for any purpose. To the extent a cited reference conflicts with the disclosure herein, the specification will control.

As used herein, the singular forms of a word also include the plural form, unless the context clearly dictates otherwise; as examples, the terms “a,” “an,” and “the” are understood to be singular or plural. By way of example, “an element” means one or more element. The term “or” means “and/or” unless the specific context indicates otherwise. All ranges, including those stated in the form of “between value X and value Y,” include the endpoints and all points in between unless the specific context indicates otherwise.

In some embodiments, the present disclosure is directed to a method of treating or preventing warm autoimmune hemolytic anemia by administering an anti-FcRn antibody or an antigen-binding fragment thereof, or by administering a pharmaceutical composition comprising at least one pharmaceutically acceptable carrier and an anti-FcRn antibody or an antigen-binding fragment thereof, to a patient in need of treatment. In some embodiments, the present disclosure is directed to a use of an anti-FcRn antibody or an antigen-binding fragment thereof in a method of treating or preventing warm autoimmune hemolytic anemia by administering the anti-FcRn antibody or antigen-binding fragment, or by administering a pharmaceutical composition comprising at least one pharmaceutically acceptable carrier and the anti-FcRn antibody or antigen-binding fragment, to a patient in need of treatment. In some embodiments, the present disclosure is directed to a use of an anti-FcRn antibody or an antigen-binding fragment thereof in the manufacture of a medicament for treating or preventing warm autoimmune hemolytic anemia. In some embodiments, the present disclosure is directed to an anti-FcRn antibody or an antigen-binding fragment thereof for use in a method of treating or preventing warm autoimmune hemolytic anemia. Pharmaceutical compositions comprising an anti-FcRn antibody or an antigen-binding fragment thereof and at least one pharmaceutically acceptable carrier are also disclosed and are useful in the therapeutic methods and uses described herein.

As used herein, the term “treat” and its cognates refer to an amelioration of a disease, disorder, or condition (e.g., warm autoimmune hemolytic anemia), or at least one discernible symptom thereof (e.g., any one or more of the signs and symptoms described herein). The term “treat” encompasses but is not limited to complete treatment or complete amelioration of one or more symptoms of warm autoimmune hemolytic anemia. In some embodiments, “treat” refers to at least partial amelioration of at least one measurable physical parameter, not necessarily discernible by the patient, e.g., a reduction in the level of at least one autoantibody and/or pathogenic antibody (e.g., a pathogenic IgG) and/or the level of total serum IgG, or an increase in the level of hemoglobin. In some embodiments, “treat” refers to inhibiting the progression of warm autoimmune hemolytic anemia, either physically (e.g., stabilization of a discernible symptom), physiologically (e.g., stabilization of a physical parameter), or both. In some embodiments, “treat” refers to slowing the progression or reversing the progression of warm autoimmune hemolytic anemia. As used herein, “treat” and its cognates also encompass delaying the onset or reducing the risk of acquiring warm autoimmune hemolytic anemia. The antibodies, antigen-binding fragments, and pharmaceutical compositions disclosed herein can also be used in the prevention or prophylaxis of warm autoimmune hemolytic anemia. For instance, a prophylactic method can comprise administering to a subject at risk of developing warm autoimmune hemolytic anemia an antibody, antigen-binding fragment, or pharmaceutical composition disclosed herein to prevent or reduce the odds developing warm autoimmune hemolytic anemia, or at least one discernible symptom thereof. In some embodiments, the disease, disorder, or condition to be treated is warm autoimmune hemolytic anemia.

The terms “subject” and “patient” are used interchangeably herein to refer to any human or non-human animal. Non-human animals include all vertebrates (e.g., mammals and non-mammals) such as any mammal. Non-limiting examples of mammals include humans, mice, rats, rabbits, dogs, monkeys, and pigs. In various embodiments, the subject is a human. In various embodiments, the subject is a human having or suspected of having warm autoimmune hemolytic anemia.

As used herein, the term “warm autoimmune hemolytic anemia” or “WAIHA” refers to an autoimmune condition defined by the presence of autoantibodies that attach to and destroy red blood cells (with or without complement activation) at temperatures equal to or greater than normal body temperature. Warm autoimmune hemolytic anemia may also be referred to as warm antibody hemolytic anemia, idiopathic warm antibody hemolytic anemia, warm antibody autoimmune hemolytic anemia, and/or warm reacting antibody disease. Generally, antibodies in warm autoimmune hemolytic anemia react optimally at 37 degrees Celsius. The most common antibody isotype involved in warm autoimmune hemolytic anemia is IgG, with a greater prevalence of IgG1 and IgG3 (Kalfa, Hematology Am. Soc. Hematol. Educ. Program 2016(1):690-7, 2016). Intravascular destruction of RBCs through complement mediated mechanisms contributes in only a minor percentage of warm autoimmune hemolytic anemia patients. In most patients, erythrocytes coated by warm reacting IgG are bound by spleen macrophages via FcRn, which can cause them to be either phagocytosed or to have part of their membrane removed in the spleen. In the latter case, such erythrocytes can form microspherocytes that are then subject to further destruction during their next passage through the spleen (Kalfa, Hematology Am. Soc. Hematol. Educ. Program 2016(1):690-7, 2016). CD8⁺ T cells, and natural killer (NK cells) may also contribute to RBC lysis through antibody dependent cell mediated cytotoxicity (ADCC).

The clinical presentation of WAIHA is typically characterized by fatigue, exertional dyspnea, pallor, and splenomegaly. Common laboratory findings include but are not limited to: decreased hemoglobin (Hb), reticulocytosis, elevated unconjugated bilirubin and lactate dehydrogenase, serum aspartate aminotransferase disproportionately higher than serum alanine aminotransferase, and decreased haptoglobin (Kalfa, Hematology Am. Soc. Hematol. Educ. Program 2016(1):690-7, 2016). Signs and symptoms of warm autoimmune hemolytic anemia can include, but are not limited to, abnormal paleness of the skin (pallor), fatigue, difficulty breathing upon exertion, dizziness, palpitations, yellowing of the skin and/or whites of the eyes (jaundice), enlargement of the spleen (splenomegaly), and enlargement of the liver (hepatomegaly). Affected individuals, especially those with a gradual onset of anemia, may also be asymptomatic and not display any signs or symptoms. Diagnosis of warm autoimmune hemolytic anemia may involve a thorough clinical evaluation, a detailed patient history, identification of characteristic symptoms, and/or a variety of tests, e.g., blood tests that measure hemoglobin and/or hematocrit. Blood tests may also show elevated levels of bilirubin in the blood and/or elevated levels of immature red blood cells (reticulocytes), which can occur when the body is forced to produce extra red blood cells to make up for those that are destroyed prematurely. In addition, specialized tests such as Coombs and/or dithiothreitol (DTT) tests may be performed. A Coombs test may be used to detect antibodies that act against red blood cells. For a Coombs test, in some embodiments, a sample of blood is taken and then exposed to the Coombs reagent. A positive Coombs test may be indicated when red blood cells clump or aggregate in the presence of the reagent. A DTT test may also be performed, e.g., to distinguish warm autoimmune hemolytic anemia caused by IgM autoantibodies from the more common form caused by IgG autoantibodies, as DTT generally reacts with IgM, but not with IgG.

In some embodiments, a patient in need of treatment or being treated for warm autoimmune hemolytic anemia is evaluated using a rating scale, e.g., any of the rating scales described herein.

In some embodiments, a patient in need of treatment or being treated for warm autoimmune hemolytic anemia is evaluated using a Functional Assessment of Chronic Illness Therapy-Fatigue (FACIT-F) scale. A FACIT-F scale is a validated scale which measures the physical, emotional and social implications of fatigue, one of the main clinical manifestations of warm autoimmune hemolytic anemia (Acaster et al., Health Qual. Life Outcomes 13(1):60-9, 2015; Webster et al., Health Qual. Life Outcomes 1(79):1-7, 2003). Scores range from 0-52, with a higher score indicating a higher quality of life. A score of less than 30 generally indicates severe fatigue.

In some embodiments, a patient in need of treatment or being treated for warm autoimmune hemolytic anemia is evaluated using a Medical Research Council (MRC) Breathlessness scale. The MRC Breathlessness scale is a questionnaire that consists of five statements about perceived breathlessness. The focus of the scale is to quantify the disability associated with breathlessness and not the severity of the breathlessness (Stenton, Occupational Med. 58:226-7, 2008). This scale has undergone iterations with the current Modified MRC Subject Version, ranging from Grade 0 (limited to no disability) to Grade 4 (severe disability). This scale has been used in patients with chronic obstructive pulmonary disease (COPD) and further stratified on patients with low hemoglobin levels, to demonstrate that anemic COPD patients can have significantly higher MRC (Ferrari et al., BMC Pulm. Med. 15:58, 2015). The scale can be self-administered by asking patients to select a phrase that best describes their condition. The score is the number that best fits the patient's level of activity.

In some embodiments, a patient in need of treatment or being treated for warm autoimmune hemolytic anemia is evaluated using a EQ-5D-3L scale. The EQ-5D-3L is a validated measurement of health-related quality of life (Devlin et al., Health Econ. 27(1):7-22, 2018; Hernandez et al., EEPRU Report: “Quality review of a proposed EQ-5D-5L value set for England” [online]). The scale consists of two components, the EQ-5D descriptive system and the EQ visual analogue scale. The descriptive system evaluates mobility, self-care, usual activities, pain/discomfort, and anxiety/depression. The scale can be self-administered by patients who select the most appropriate statement within each category. A lower score corresponds to a better quality of life. The EQ VAS records the patient's self-rated health on a vertical visual analogue scale where the endpoints are labeled ‘Best imaginable health state’ (100) and ‘Worst imaginable health state’ (0). The patient can select any number from 0-100.

In some embodiments, a patient in need of treatment for warm autoimmune hemolytic anemia exhibits one or more signs and symptoms of warm autoimmune hemolytic anemia (e.g., pallor, fatigue, jaundice, and/or enlargement of the spleen) and/or has been diagnosed with any form of the condition by a treating clinician. In some embodiments, a patient in need of treatment for warm autoimmune hemolytic anemia (or a sample from the patient) has a detectable level of an anti-red blood cell IgG (anti-RBC IgG), i.e., an IgG capable of binding to at least one red blood cell. In some embodiments, the anti-RBC IgG acts and/or contributes to disease pathogenesis by complement fixation (CF). In some embodiments, the anti-RBC IgG acts and/or contributes to disease pathogenesis by engagement of one or more Fc receptors (e.g., activation of a patient's innate immune system, including, e.g., cytokine release and/or phagocytosis). In some embodiments, the anti-RBC IgG acts and/or contributes to disease pathogenesis by activation of a patient's innate immune system, including, e.g., cytokine release and/or phagocytosis. In some embodiments, the anti-RBC IgG is present in the patient's blood. In some embodiments, the anti-RBC IgG is an anti-RBC IgG1. In some embodiments, the anti-RBC IgG is an anti-RBC IgG2. In some embodiments, the anti-RBC IgG is an anti-RBC IgG3. In some embodiments, the anti-RBC IgG is an anti-RBC IgG4.

One embodiment is a method of treating or preventing warm autoimmune hemolytic anemia in a patient in need thereof, comprising administering to the patient (i) a therapeutically effective amount of an anti-FcRn antibody or an antigen-binding fragment thereof; or (ii) a pharmaceutical composition comprising at least one pharmaceutically acceptable carrier and a therapeutically effective amount of an anti-FcRn antibody or an antigen-binding fragment thereof.

Another embodiment is an anti-FcRn antibody or an antigen-binding fragment thereof for use in a method of treating or preventing warm autoimmune hemolytic anemia in a patient in need thereof, the method comprising administering to the patient (i) a therapeutically effective amount of the antibody or antigen-binding fragment, or (ii) a pharmaceutical composition comprising at least one pharmaceutically acceptable carrier and a therapeutically effective amount of the antibody or antigen-binding fragment.

Another embodiment is a use of an anti-FcRn antibody or an antigen-binding fragment thereof in a method of treating or preventing warm autoimmune hemolytic anemia in a patient in need thereof, comprising administering to the patient (i) a therapeutically effective amount of the antibody or antigen-binding fragment; or (ii) a pharmaceutical composition comprising at least one pharmaceutically acceptable carrier and a therapeutically effective amount of the antibody or antigen-binding fragment.

Another embodiment is a use of an anti-FcRn antibody or an antigen-binding fragment thereof in the manufacture of a medicament for treating or preventing warm autoimmune hemolytic anemia in a patient in need thereof.

In various embodiments of the therapeutic methods, uses, and compositions disclosed herein, an anti-FcRn antibody or antigen-binding fragment acts as a non-competitive inhibitor of IgG in binding to FcRn. In various embodiments, the binding of the antibody or antigen-binding fragment to FcRn inhibits the binding of at least one autoantibody and/or pathogenic antibody to FcRn. In various embodiments, such inhibition promotes clearance (i.e., removal) of the at least one autoantibody and/or pathogenic antibody from the body of the subject. In various embodiments, such inhibition reduces the half-life of the at least one autoantibody and/or pathogenic antibody. In various embodiments, such inhibition reduces the level of the at least one autoantibody and/or pathogenic antibody in the subject and/or in a sample from the subject. In various embodiments, a reduction in the level of the at least one autoantibody and/or pathogenic antibody results in and/or correlates with an improvement in at least one clinical parameter of warm autoimmune hemolytic anemia.

As used herein, the term “autoantibody” refers to an antibody produced by an organism's immune system that is directed against one or more of the organism's own proteins, tissues, and/or organs. For instance, one or more autoantibodies may be produced by a human patient's immune system when it fails to distinguish between “self” and “non-self.” In some embodiments, the autoantibody is a pathogenic antibody (e.g., a pathogenic IgG, e.g., a pathogenic IgG1, IgG2, IgG3, or IgG4). The term “pathogenic antibody,” as used herein, refers to an antibody (e.g., an autoantibody) that contributes to the pathogenesis of and/or causes one or more diseases, disorders, or conditions (e.g., warm autoimmune hemolytic anemia).

In some embodiments, the pathogenic antibody is a pathogenic IgG (e.g., a pathogenic IgG1, IgG2, IgG3, or IgG4). In some embodiments, the pathogenic antibody and/or pathogenic IgG is an anti-red blood cell IgG (anti-RBC IgG).

In some embodiments, the autoantibody and/or pathogenic antibody is an autoantibody capable of binding to a red blood cell (RBC) (i.e., at least one red blood cell antigen). In some embodiments, the autoantibody and/or pathogenic antibody is an anti-red blood cell IgG (anti-RBC IgG). In some embodiments, the autoantibody and/or pathogenic antibody is an anti-red blood cell IgG1 (anti-RBC IgG1). In some embodiments, the autoantibody and/or pathogenic antibody is an anti-red blood cell IgG2 (anti-RBC IgG2). In some embodiments, the autoantibody and/or pathogenic antibody is an anti-red blood cell IgG3 (anti-RBC IgG3). In some embodiments, the autoantibody and/or pathogenic antibody is an anti-red blood cell IgG4 (anti-RBC IgG4). In some embodiments, treatment of a patient with an antibody, antigen-binding fragment, or pharmaceutical composition described herein, e.g., using a method described herein, reduces the level of an anti-RBC IgG (e.g., an anti-RBC IgG1, an anti-RBC IgG2, an anti-RBC IgG3, and/or an anti-RBC IgG4) by least about 20%, about 25%, about 30%, about 35%, about 40%, about 45%, about 50%, i.e., relative to the level of the anti-RBC IgG prior to treatment.

In some embodiments, the autoantibody and/or pathogenic antibody is an IgG, IgM, IgA, IgD, or IgE. In some embodiments, the autoantibody and/or pathogenic antibody is an IgG (e.g., a pathogenic IgG). In some embodiments, the autoantibody and/or pathogenic antibody is an IgG1, IgG2, IgG3, or IgG4. In some embodiments, the autoantibody and/or pathogenic antibody is an IgG1 (e.g., a pathogenic IgG1, e.g., an anti-RBC IgG1). In some embodiments, the autoantibody and/or pathogenic antibody is an IgG2 (e.g., a pathogenic IgG2, e.g., an anti-RBC IgG2). In some embodiments, the autoantibody and/or pathogenic antibody is an IgG3 (e.g., a pathogenic IgG3, e.g., an anti-RBC IgG3). In some embodiments, the autoantibody and/or pathogenic antibody is an IgG4 (e.g., a pathogenic IgG4, e.g., an anti-RBC IgG4). In some embodiments, the autoantibody is a pathogenic antibody.

In various embodiments of the therapeutic methods, uses, and compositions disclosed herein, an anti-FcRn antibody or antigen-binding fragment can non-competitively inhibit the binding of at least one autoantibody and/or pathogenic antibody (e.g., at least one IgG) to FcRn at physiological pH (i.e., pH 7.0-7.4). Without wishing to be bound by theory, it is believed that FcRn binds to its ligand (i.e., IgG) and does not substantially show affinity for IgG at physiological pH rather than acidic pH. Thus, in various embodiments, at physiological pH, the anti-FcRn antibody or antigen-binding fragment may act as a non-competitive inhibitor of the binding of IgG to FcRn, and the binding of the anti-FcRn antibody or antigen-binding fragment to FcRn is not influenced by the presence of IgG. Thus, in various embodiments, the anti-FcRn antibody or antigen-binding fragment that binds specifically to FcRn non-competitively with IgG in a pH-independent manner has an advantage over conventional competitive inhibitors (i.e., antibodies that bind to FcRn competitively with IgG) in that it can provide therapeutic or prophylactic effects even at significantly low concentrations by the FcRn-mediated signaling of IgG. In addition, in various embodiments, in the procedure of intracellular migration in a state bound to FcRn, the anti-FcRn antibody or antigen-binding fragment can maintain its binding to FcRn with an affinity higher than IgG in blood. Thus, in various embodiments, the anti-FcRn antibody or antigen-binding fragment can inhibit the binding of IgG to FcRn even in endosomes that are acidic pH environments in which IgG can bind to FcRn, thereby promoting the clearance of IgG. In various embodiments, the anti-FcRn antibody or antigen-binding fragment is RVT-1401 (also referred to herein as HL161BKN). In some embodiments, the antibody or antigen-binding fragment is RVT-1401, or an antigen-binding fragment thereof. In some embodiments, the antibody or antigen-binding fragment comprises three heavy chain CDR amino acid sequences of SEQ ID No: 27 (HCDR1), SEQ ID No: 28 (HCDR2), SEQ ID No: 29 (HCDR3); and three light chain CDR amino acid sequences of SEQ ID No: 30 (LCDR1), SEQ ID No: 31 (LCDR2), SEQ ID No: 32 (LCDR3). In some embodiments, the antibody or antigen-binding fragment comprises a heavy chain variable region amino acid sequence of SEQ ID No: 6; and a light chain variable region amino acid sequence of SEQ ID No: 16. In some embodiments, the antibody or antigen-binding fragment comprises a heavy chain amino acid sequence of SEQ ID No: 46; and a light chain amino acid sequence of SEQ ID No: 48.

Binding “affinity” refers to the strength of interaction between antibody and antigen at single antigenic sites. Within each antigenic site, the variable region of the antibody “arm” interacts through weak non-covalent forces with the antigen at numerous sites. In general, the more interactions, the higher the affinity.

As used herein, the term “specific,” “specifically binds,” and “binds specifically” refers to a binding reaction between an antibody or an antigen-binding fragment thereof (e.g., an anti-FcRn antibody or an antigen-binding fragment thereof) and a target antigen (e.g., FcRn) in a heterogeneous population of proteins and other biologics. Antibodies can be tested for specificity of binding by comparing binding to an appropriate antigen with binding to an alternate antigen or antigen mixture under a given set of conditions. If the antibody binds to the appropriate antigen with at least 2 times, at least 5 times, or at least 10 times (or more) higher affinity than to the alternate antigen or antigen mixture, then it is considered to be specific.

A“specific antibody” or a “target-specific antibody” is one that only binds the target antigen (e.g., FcRn), but does not bind (or exhibits minimal binding) to other antigens. In some embodiments, an antibody or an antigen-binding fragment thereof that specifically binds the target antigen (e.g., FcRn) has a K_{D} of less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M at pH 6.0 or pH 7.4. In some embodiments, the K_{D} is about 0.01 nM to about 2 nM at pH 6.0 or pH 7.4. In some embodiments, the K_{D} is about 300 pM or less to about 2 nM or less at pH 7.4. In some embodiments, the K_{D} is about 2 nM or less to 900 pM or less at pH 6.0.

As used herein, the term “K_{D}” refers to the equilibrium dissociation constant for antibody-antigen binding, which is obtained from the ratio of k_{d} to kₐ (i.e., k_{d}/kₐ) and is generally expressed as a molar concentration (M). The term “k_{assoc}” or “kₐ” refers to the association rate of a particular antibody-antigen interaction, whereas the term “k_{dis}” or “k_{d}” refers to the dissociation rate of a particular antibody-antigen interaction. The measurement of k_{d} and/or kₐ can be performed at 25° C. or 37° C. K_{D} values for antibodies and antigen-binding fragments can be determined using methods well established in the art (see, e.g., Pollard, Mol. Biol. Cell 21(23):4061-7, 2010). In some embodiments, the K_{D} is measured by direct binding and/or competition binding assays (e.g., surface plasmon resonance and/or competition ELISA). In some embodiments, the K_{D} is measured by surface plasmon resonance (e.g., human FcRn-immobilized surface plasmon resonance). In some embodiments, the K_{D} of an anti-FcRn antibody or antigen-binding fragment disclosed herein is measured by human FcRn-immobilized surface plasmon resonance.

In some embodiments of the therapeutic methods, uses, and compositions disclosed herein, the anti-FcRn antibody or antigen-binding fragment has a K_{D} (dissociation constant) of about 0.01 nM to 2 nM at pH 6.0 and pH 7.4, as determined by, e.g., surface plasmon resonance. In some embodiments, the anti-FcRn antibody or antigen-binding fragment has a K_{D} from about 300 pM or less to about 2 nM or less at pH 7.4 and/or has a K_{D} from about 2 nM or less to about 900 pM or less at pH 6.0, as determined by, e.g., surface plasmon resonance. In some embodiments, the anti-FcRn antibody or antigen-binding fragment binds to the outside of cells and when bound maintains its binding to endosomes. In some embodiments, the anti-FcRn antibody or antigen-binding fragment effectively blocks the binding of one or more autoantibodies to FcRn (e.g., human FcRn), as determined by, e.g., a blocking assay performed using human FcRn-expressing cells and FACS.

As used herein, the term “anti-FcRn antibody” or “antibody that binds specifically to FcRn” refers to any form of an antibody or an antigen-binding fragment thereof that binds specifically to FcRn, e.g., those binding with a K_{D} of less than 2 nM at pH 6.0 or pH 7.4, as determined by, e.g., surface plasmon resonance (e.g., human FcRn-immobilized surface plasmon resonance). The term encompasses monoclonal antibodies (including full-length monoclonal antibodies), polyclonal antibodies, and biologically functional fragments so long as they bind specifically to FcRn.

In some embodiments of the therapeutic methods, uses, and compositions disclosed herein, the anti-FcRn antibody or antigen-binding fragment comprises:

CDR1 comprising an amino acid sequence that is at least 90% identical to one or more amino acid sequences selected from the group consisting of SEQ ID Nos: 21, 24, 27, 30, 33, 36, 39, and 42;

CDR2 comprising an amino acid sequence that is at least 90% identical to one or more amino acid sequences selected from the group consisting of SEQ ID Nos: 22, 25, 28, 31, 34, 37, 40, and 43; and

CDR3 comprising an amino acid sequence that is at least 90% identical to one or more amino acid sequences selected from the group consisting of SEQ ID Nos: 23, 26, 29, 32, 35, 38, 41, and 44.

In some embodiments of the therapeutic methods, uses, and compositions disclosed herein, the anti-FcRn antibody or antigen-binding fragment comprises:

CDR1 comprising an amino acid sequence that is at least 91%, at least 92%, at least 93%, at least 94%, at least 95%, at least 96%, at least 97%, at least 98%, or at least 99% identical to one or more amino acid sequences selected from the group consisting of SEQ ID Nos: 21, 24, 27, 30, 33, 36, 39, and 42;

CDR2 comprising an amino acid sequence that is at least 91%, at least 92%, at least 93%, at least 94%, at least 95%, at least 96%, at least 97%, at least 98%, or at least 99% identical to one or more amino acid sequences selected from the group consisting of SEQ ID Nos: 22, 25, 28, 31, 34, 37, 40, and 43; and

CDR3 comprising an amino acid sequence that is at least 91%, at least 92%, at least 93%, at least 94%, at least 95%, at least 96%, at least 97%, at least 98%, or at least 99% identical to one or more amino acid sequences selected from the group consisting of SEQ ID Nos: 23, 26, 29, 32, 35, 38, 41, and 44.

In some embodiments of the therapeutic methods, uses, and compositions disclosed herein, the anti-FcRn antibody or antigen-binding fragment may comprise one or more amino acid deletions, additions, or substitutions in the amino acid sequences described herein.

In some embodiments of the therapeutic methods, uses, and compositions disclosed herein, the anti-FcRn antibody or antigen-binding fragment may comprise amino acid sequences identical to or having homology with the amino acid sequences described herein. The term “identity” or “homology” refers to a relationship between the sequences of two or more polypeptides, as determined by comparing the sequences. The term “identity” also means the degree of sequence relatedness between the polypeptides, as determined by the number of matches between strings of two or more amino acid residues. The percent “identity” between the two sequences is a function of the number of identical positions shared by the sequences (i.e., percent identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Additionally, or alternatively, the amino acid sequences disclosed herein can further be used as a “query sequence” to perform a search against public databases to, for example, identify related sequences. For example, such searches can be performed using the BLAST program of Altschul et al. (J. Mol. Biol. 215:403-10, 1990).

Two sequences are “substantially identical” if two sequences have a specified percentage of amino acid residues that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 10 amino acids in length, or over a region that is about 20, 50, 200 or more amino acids in length. In some embodiments, the anti-FcRn antibodies and antigen-binding fragments described herein comprise at least one amino acid sequence that is at least 90% identical to a sequence selected from the group consisting of SEQ ID Nos: 2, 4, 6, 8, 10, 12, 14, 16, 18, and 20-48. In some embodiments, the anti-FcRn antibodies and antigen-binding fragments described herein comprise at least one amino acid sequence that is at least 91%, at least 92%, at least 93%, at least 94%, at least 95%, at least 96%, at least 97%, at least 98%, or at least 99% identical to a sequence selected from the group consisting of SEQ ID Nos: 2, 4, 6, 8, 10, 12, 14, 16, 18, and 20-48.

In some embodiments, the antibody or antigen-binding fragment comprises a heavy chain variable region comprising:

CDR1 comprising an amino acid sequence of SEQ ID No: 21, CDR2 comprising an amino acid sequence of SEQ ID No: 22, and CDR3 comprising an amino acid sequence of SEQ ID No: 23;

CDR1 comprising an amino acid sequence of SEQ ID No: 27, CDR2 comprising an amino acid sequence of SEQ ID No: 28, and CDR3 comprising an amino acid sequence of SEQ ID No: 29;

CDR1 comprising an amino acid sequence of SEQ ID No: 33, CDR2 comprising an amino acid sequence of SEQ ID No: 34, and CDR3 comprising an amino acid sequence of SEQ ID No: 35; or

CDR1 comprising an amino acid sequence of SEQ ID No: 39, CDR2 comprising an amino acid sequence of SEQ ID No: 40, and CDR3 comprising an amino acid sequence of SEQ ID No: 41.

In some embodiments, the antibody or antigen-binding fragment comprises a light chain variable region comprising:

CDR1 comprising an amino acid sequence of SEQ ID No: 24, CDR2 comprising an amino acid sequence of SEQ ID No: 25, and CDR3 comprising an amino acid sequence of SEQ ID No: 26;

CDR1 comprising an amino acid sequence of SEQ ID No: 30, CDR2 comprising an amino acid sequence of SEQ ID No: 31, and CDR3 comprising an amino acid sequence of SEQ ID No: 32;

CDR1 comprising an amino acid sequence of SEQ ID No: 36, CDR2 comprising an amino acid sequence of SEQ ID No: 37, and CDR3 comprising an amino acid sequence of SEQ ID No: 38; or

CDR1 comprising an amino acid sequence of SEQ ID No: 42, CDR2 comprising an amino acid sequence of SEQ ID No: 43, and CDR3 comprising an amino acid sequence of SEQ ID No: 44.

In some embodiments, the antibody or antigen-binding fragment comprises one or more heavy chain variable regions and one or more light chain variable regions selected from the group consisting of:

heavy chain variable region comprising CDR1 comprising an amino acid sequence of SEQ ID No: 21 (HCDR1), CDR2 comprising an amino acid sequence of SEQ ID No: 22 (HCDR2), and CDR3 comprising an amino acid sequence of SEQ ID No: 23 (HCDR3); and light chain variable region comprising CDR1 comprising an amino acid sequence of SEQ ID No: 24 (LCDR1), CDR2 comprising an amino acid sequence of SEQ ID No: 25 (LCDR2), and CDR3 comprising an amino acid sequence of SEQ ID No: 26 (LCDR3);

heavy chain variable region comprising CDR1 comprising an amino acid sequence of SEQ ID No: 27 (HCDR1), CDR2 comprising an amino acid sequence of SEQ ID No: 28 (HCDR2), and CDR3 comprising an amino acid sequence of SEQ ID No: 29 (HCDR3); and light chain variable region comprising CDR1 comprising an amino acid sequence of SEQ ID No: 30 (LCDR1), CDR2 comprising an amino acid sequence of SEQ ID No: 31 (LCDR2), and CDR3 comprising an amino acid sequence of SEQ ID No: 32 (LCDR3);

heavy chain variable region comprising CDR1 comprising an amino acid sequence of SEQ ID No: 33 (HCDR1), CDR2 comprising an amino acid sequence of SEQ ID No: 34 (HCDR2), and CDR3 comprising an amino acid sequence of SEQ ID No: 35 (HCDR3); and light chain variable region comprising CDR1 comprising an amino acid sequence of SEQ ID No: 36 (LCDR1), CDR2 comprising an amino acid sequence of SEQ ID No: 37 (LCDR2), and CDR3 comprising an amino acid sequence of SEQ ID No: 38 (LCDR3); and

heavy chain variable region comprising CDR1 comprising an amino acid sequence of SEQ ID No: 39 (HCDR1), CDR2 comprising an amino acid sequence of SEQ ID No: 40 (HCDR2), and CDR3 comprising an amino acid sequence of SEQ ID No: 41 (HCDR3); and light chain variable region comprising CDR1 comprising an amino acid sequence of SEQ ID No: 42 (LCDR1), CDR2 comprising an amino acid sequence of SEQ ID No: 43 (LCDR2), and CDR3 comprising an amino acid sequence of SEQ ID No: 44 (LCDR3).

In some embodiments, the antibody or antigen-binding fragment comprises one or more heavy chain variable regions and/or one or more light chain variable regions comprising one or more amino acid sequences selected from the group consisting of amino acid sequences of SEQ ID Nos: 2, 4, 6, 8, 10, 12, 14, 16, 18, and 20.

In some embodiments, the antibody or antigen-binding fragment comprises heavy chain variable region comprising an amino acid sequence of SEQ ID Nos: 2, 4, 6, 8, or 10, and/or light chain variable region comprising an amino acid sequence of SEQ ID Nos: 12, 14, 16, 18, or 20.

In some embodiments, the antibody or antigen-binding fragment comprises one or more heavy chain variable regions and one or more light chain variable regions selected from the group consisting of:

heavy chain variable region comprising an amino acid sequence of SEQ ID No: 2 and light chain variable region comprising an amino acid sequence of SEQ ID No: 12;

heavy chain variable region comprising an amino acid sequence of SEQ ID No: 4 and light chain variable region comprising an amino acid sequence of SEQ ID No: 14;

heavy chain variable region comprising an amino acid sequence of SEQ ID No: 6 and light chain variable region comprising an amino acid sequence of SEQ ID No: 16;

heavy chain variable region comprising an amino acid sequence of SEQ ID No: 8 and light chain variable region comprising an amino acid sequence of SEQ ID No: 18; and

heavy chain variable region comprising an amino acid sequence of SEQ ID No: 10 and light chain variable region comprising an amino acid sequence of SEQ ID No: 20.

The terms “fragment,” “antibody fragment,” and “antigen-binding fragment,” as used herein in reference to an antibody, all refer to one or more fragments of a full-length antibody that retain the ability to specifically bind to the target antigen (e.g., FcRn) and/or provide a function of the full-length antibody (e.g., non-competitive interference with the binding of IgG to FcRn). Antigen-binding fragments can also be present in larger macromolecules, e.g., bispecific, trispecific, and multispecific antibodies. Examples of antigen-binding fragments include, but are not limited to, single-chain antibodies, bispecific, trispecific, and multispecific antibodies such as diabodies, triabodies and tetrabodies, Fab fragments, F(ab′)₂ fragments, Fd, scFv, domain antibodies, dual-specific antibodies, minibodies, scap (sterol regulatory binding protein cleavage activating protein), chelating recombinant antibodies, tribodies or bibodies, intrabodies, nanobodies, small modular immunopharmaceuticals (SMIP), binding-domain immunoglobulin fusion proteins, camelized antibodies, VHH containing antibodies, IgD antibodies, IgE antibodies, IgM antibodies, IgG1 antibodies, IgG2 antibodies, IgG3 antibodies, IgG4 antibodies, derivatives in antibody constant regions, and synthetic antibodies based on protein scaffolds that have the ability to bind to FcRn. In some embodiments, an antigen-binding fragment shows the same or similar properties as those of the full-length antibody. Without limitation, an antigen-binding fragment can be produced by any suitable method known in the art. For instance, the various antigen-binding fragments described herein may be produced by enzymatic or chemical modification of full-length antibodies, synthesized de novo using recombinant DNA methodologies (e.g., scFv), or identified using phage display libraries (see, e.g., Pini and Bracci, Curr. Protein Pept. Sci. 1(2):155-69, 2000). Antigen-binding fragments may be screened for utility (e.g., specificity, binding affinity, activity) in the same manner as are full-length antibodies.

In addition, antibodies or antigen-binding fragments having a mutation in the variable and/or constant region may be used in the therapeutic methods, uses, and compositions described herein. Examples of such antibodies or antigen-binding fragments include antibodies having a conservative substitution of an amino acid residue in the variable region and/or constant region. As used herein, the term “conservative substitution” refers to a substitution with another amino acid residue having properties similar to those of the original amino acid residue. For example, lysine, arginine and histidine have similar properties in that they have a basic side-chain, and aspartic acid and glutamic acid have similar properties in that they have an acidic side chain. In addition, glycine, asparagine, glutamine, serine, threonine, tyrosine, cysteine and tryptophan have similar properties in that they have an uncharged polar side-chain, and alanine, valine, leucine, threonine, isoleucine, proline, phenylalanine and methionine have similar properties in that they have a non-polar side-chain. Also, tyrosine, phenylalanine, tryptophan and histidine have similar properties in that they have an aromatic side-chain. Thus, it will be obvious to those skilled in the art that, even when substitution of amino acid residues in groups showing similar properties as described above occurs, it will likely show no significant change in the properties of the antibody or antigen-binding fragment.

In addition, in some embodiments, the antibody or antigen-binding fragment may be conjugated to another substance (e.g., a therapeutic agent or a detectable label). Substances that may be conjugated to, or administered in combination with, an antibody or antigen-binding fragment described herein include but are not limited to therapeutic agents that are generally used for the treatment of warm autoimmune hemolytic anemia (e.g., a standard-of-care agent, e.g., any one or more of the standard-of-care agents described and/or incorporated by reference herein); substances that are capable of inhibiting the activity of FcRn; and moieties that may be physically associated with the antibody or antigen-binding fragment, e.g., to improve its stabilization and/or retention in circulation, e.g., in blood, serum, lymph, or other tissues. For example, the antibody or antigen-binding fragment can be associated with a polymer, e.g., a non-antigenic polymer such as polyalkylene oxide or polyethylene oxide. Suitable polymers will vary substantially by weight. Polymers having molecular number average weights ranging from about 200 to about 35,000 (or about 1,000 to about 15,000, and 2,000 to about 12,500) can be used. For example, the antibody or antigen-binding fragment can be conjugated to water soluble polymers, e.g., hydrophilic polyvinyl polymers, e.g., polyvinylalcohol and polyvinylpyrrolidone. Non-limiting examples of such polymers include, but are not limited to, polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In various embodiments of the therapeutic methods, uses, and compositions disclosed herein, the antibody or antigen-binding fragment comprises a heavy chain variable region comprising an amino acid sequence of SEQ ID No: 27 (HCDR1), an amino acid sequence of SEQ ID No: 28 (HCDR2), and an amino acid sequence of SEQ ID No: 29 (HCDR3); and a light chain variable region comprising an amino acid sequence of SEQ ID No: 30 (LCDR1), an amino acid sequence of SEQ ID No: 31 (LCDR2), and an amino acid sequence of SEQ ID No: 32 (LCDR3).

In various embodiments, the antibody or antigen-binding fragment comprises a heavy chain variable region comprising an amino acid sequence of SEQ ID No: 6; and a light chain variable region comprising an amino acid sequence of SEQ ID No: 16. In various embodiments, the antibody or antigen-binding fragment comprises a heavy chain variable region comprising an amino acid sequence that is at least 90% identical to SEQ ID No: 6; and a light chain variable region comprising an amino acid sequence that is at least 90% identical to SEQ ID No: 16. In various embodiments, the antibody or antigen-binding fragment comprises a heavy chain variable region comprising an amino acid sequence of SEQ ID No: 4; and a light chain variable region comprising an amino acid sequence of SEQ ID No: 14. In various embodiments, the antibody or antigen-binding fragment comprises a heavy chain variable region comprising an amino acid sequence that is at least 90% identical to SEQ ID No: 4; and a light chain variable region comprising an amino acid sequence that is at least 90% identical to SEQ ID No: 14. In various embodiments, the antibody or antigen-binding fragment binds to FcRn with a K_{D} (dissociation constant) of 0.01 nM to 2 nM at pH 6.0 or pH 7.4, as measured by, e.g., surface plasmon resonance.

In various embodiments of the therapeutic methods, uses, and compositions disclosed herein, the antibody or antigen-binding fragment comprises a heavy chain variable region comprising an amino acid sequence of SEQ ID No: 21 (HCDR1), an amino acid sequence of SEQ ID No: 22 (HCDR2), and an amino acid sequence of SEQ ID No: 23 (HCDR3); and a light chain variable region comprising an amino acid sequence of SEQ ID No: 24 (LCDR1), an amino acid sequence of SEQ ID No: 25 (LCDR2), and an amino acid sequence of SEQ ID No: 26 (LCDR3).

In various embodiments, the antibody or antigen-binding fragment comprises a heavy chain variable region comprising an amino acid sequence of SEQ ID No: 2; and a light chain variable region comprising an amino acid sequence of SEQ ID No: 12. In various embodiments, the antibody or antigen-binding fragment comprises a heavy chain variable region comprising an amino acid sequence that is at least 90% identical to SEQ ID No: 2; and a light chain variable region comprising an amino acid sequence that is at least 90% identical to SEQ ID No: 12. In various embodiments, the antibody or antigen-binding fragment binds to FcRn with a K_{D} of 0.01 nM to 2 nM at pH 6.0 or pH 7.4, as measured by, e.g., surface plasmon resonance.

In various embodiments, the antibody or antigen-binding fragment comprises a heavy chain amino acid sequence of SEQ ID No: 46, or a sequence that is at least 90% identical to SEQ ID No: 46. In various embodiments, the antibody or antigen-binding fragment comprises a light chain amino acid sequence of SEQ ID No: 48, or a sequence that is at least 90% identical to SEQ ID No: 48. In various embodiments, the antibody or antigen-binding fragment comprises a heavy chain amino acid sequence of SEQ ID No: 46, and a light chain amino acid sequence of SEQ ID No: 48. In various embodiments, the antibody or antigen-binding fragment comprises a heavy chain amino acid sequence that is at least 95%, at least 96%, at least 97%, at least 98%, at least 99%, or 100% identical to SEQ ID No: 46, and a light chain amino acid sequence that is at least 95%, at least 96%, at least 97%, at least 98%, at least 99%, or 100% identical to SEQ ID No: 48.

RVT-1401 (also referred to herein as HL161BKN) is an example of an anti-FcRn antibody. In some embodiments, the antibody or antigen-binding fragment is RVT-1401, or an antigen-binding fragment thereof. In some embodiments, the antibody or antigen-binding fragment comprises the three heavy chain CDR amino acid sequences of RVT-1401 (HCDR1 (SEQ ID No: 27), HCDR2 (SEQ ID No: 28), HCDR3 (SEQ ID No: 29)); and the three light chain CDR amino acid sequences of RVT-1401 (LCDR1 (SEQ ID No: 30), LCDR2 (SEQ ID No: 31), LCDR3 (SEQ ID No: 32)). In some embodiments, the antibody or antigen-binding fragment comprises the heavy chain variable region amino acid sequence of RVT-1401 (SEQ ID No: 6); and the light chain variable region amino acid sequence of RVT-1401 (SEQ ID No: 16). In some embodiments, the antibody or antigen-binding fragment comprises the heavy chain amino acid sequence of RVT-1401 (SEQ ID No: 46); and the light chain amino acid sequence of RVT-1401 (SEQ ID No: 48).

In various embodiments of the therapeutic methods and uses disclosed herein, the antibody or antigen-binding fragment is administered alone. In various embodiments, the antibody or antigen-binding fragment is administered in combination with at least one additional therapeutic agent. In various embodiments, the at least one additional therapeutic agent may comprise or consist of a standard-of-care agent for warm autoimmune hemolytic anemia.

Administered “in combination” or “co-administration,” as used herein, means that two or more different treatments are delivered to a subject during the subject's affliction with warm autoimmune hemolytic anemia. For example, in some embodiments, the two or more treatments are delivered after the subject has been diagnosed with the disease, and before the disease has been cured or eliminated, or when a subject is identified as being at risk but before the subject has developed symptoms of the disease. In some embodiments, the delivery of one treatment is still occurring when the delivery of the second treatment begins, so that there is overlap. In some embodiments, the first and second treatment are initiated at the same time. These types of delivery are sometimes referred to herein as “simultaneous,” “concurrent,” or “concomitant” delivery. In other embodiments, the delivery of one treatment ends before delivery of the second treatment begins. This type of delivery is sometimes referred to herein as “successive” or “sequential” delivery. In some embodiments, the antibody or antigen-binding fragment and the at least at one additional therapeutic agent are administered simultaneously. In some embodiments, the antibody or antigen-binding fragment and the at least at one additional therapeutic agent are administered sequentially.

In some embodiments, the two treatments (e.g., an anti-FcRn antibody or antigen-binding fragment and a second therapeutic agent) are comprised in the same composition. Such compositions may be administered in any appropriate form and by any suitable route. In other embodiments, the two treatments (e.g., an anti-FcRn antibody or antigen-binding fragment and a second therapeutic agent) are administered in separate compositions, in any appropriate form and by any suitable route. For example, a composition comprising an anti-FcRn antibody or antigen-binding fragment and a composition comprising a second therapeutic agent (e.g., a standard-of-care agent for warm autoimmune hemolytic anemia) may be administered concurrently or sequentially, in any order at different points in time; in either case, they should be administered sufficiently close in time so as to provide the desired therapeutic or prophylactic effect.

The term “agent,” as used herein, refers to a chemical compound, a mixture of chemical compounds, a biological macromolecule, or an extract made from biological materials. The term “therapeutic agent” or “drug” refers to an agent that is capable of modulating a biological process and/or has biological activity. The anti-FcRn antibodies and antigen-binding fragments described herein are examples of therapeutic agents.

As used herein, the term “standard-of-care agent” refers to any therapeutic agent or other form of therapy that is accepted as a proper treatment for a certain type of disease (e.g., warm autoimmune hemolytic anemia). The term “standard dosage” or “standard dosing regimen,” as used herein, refers to any usual or routine dosing regimen for a therapeutic agent, e.g., a regimen proposed by the manufacturer, approved by regulatory authorities, or otherwise tested in human subjects to meet the average patient's needs.

One example of a standard-of-care agent for warm autoimmune hemolytic anemia is IVIG. In some embodiments, a standard dosing regimen for IVIG comprises or consists of: IVIG 1 g/kg/day for 2 days. Another example of a standard-of-care agent for warm autoimmune hemolytic anemia is one or more corticosteroids (e.g., prednisone). In some embodiments, a standard dosing regimen for one or more corticosteroids (e.g., prednisone) comprises or consists of: prednisone 1.0-1.5 mg/kg/day for 1-3 weeks until hemoglobin levels greater than 10 g/dL are achieved; and subsequent doses of prednisone slowly tapering off at 10-15 mg weekly to a daily dose of 20-30 mg, then by 5 mg every 1-2 weeks to a dose of 15 mg, and then by 2.5 mg every 2 weeks with the ultimate aim of withdrawing the drug. Additional standard-of-care agents for warm autoimmune hemolytic anemia, as well as standard dosing regimens for such agents, are known in the art and disclosed in, e.g., Kalfa, Hematology Am. Soc. Hematol. Educ. Program 2016(1):690-7, 2016; and Zanella and Barcellini, Haematologica 99(10):1547-54, 2014, which are both incorporated herein by reference for such agents and dosing regimens.

The anti-FcRn antibodies and antigen-binding fragments described herein may be administered in combination with any of the exemplary standard-of-care agents described and/or incorporated by reference herein.

Also provided herein are pharmaceutical compositions comprising the anti-FcRn antibody or an antigen-binding fragment thereof formulated together with at least one pharmaceutically acceptable carrier. The compositions may also contain one or more additional therapeutic agents that are suitable for treating or preventing, for example, warm autoimmune hemolytic anemia (e.g., a standard-of-care agent for warm autoimmune hemolytic anemia). Methods of formulating pharmaceutical compositions and suitable formulations are known in the art (see, e.g., “Remington's Pharmaceutical Sciences,” Mack Publishing Co., Easton, Pa.). Appropriate formulation may depend on the route of administration.

As used herein, a “pharmaceutical composition” refers to a preparation of an anti-FcRn antibody or an antigen-binding fragment thereof in addition to other components suitable for administration to a patient, such as a pharmaceutically acceptable carrier and/or excipient. The pharmaceutical compositions provided herein may be suitable for administration in vitro and/or in vivo. In some embodiments, the pharmaceutical compositions provided herein are in such form as to permit administration and subsequently provide the intended biological activity of the active ingredient(s) and/or to achieve a therapeutic effect. The pharmaceutical compositions provided herein preferably contain no additional components which are unacceptably toxic to a subject to which the formulation would be administered.

As used herein, the terms “pharmaceutically acceptable carrier” and “physiologically acceptable carrier,” which may be used interchangeably, refer to a carrier, diluent, or excipient that does not cause significant irritation to a subject and does not abrogate the biological activity and properties of the administered antibody or antigen-binding fragment. Thus, pharmaceutically acceptable carriers should be compatible with the active ingredient such as the antibody or an antigen-binding fragment thereof and may include physiological saline, sterile water, Ringer's solution, buffered saline, dextrose solution, maltodextrin solution, glycerol, ethanol, or a mixture of two or more thereof. Pharmaceutically acceptable carriers may also enhance or stabilize the composition, or can be used to facilitate preparation of the composition. Pharmaceutically acceptable carriers can include other conventional additives, such as antioxidants, buffers, solvents, bacteriostatic agents, dispersion media, coatings, antibacterial and antifungal agents, isotonic and absorption delaying agents, and the like that are physiologically compatible. The carrier may be selected to minimize adverse side effects in the subject, and/or to minimize degradation of the active ingredient(s).

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated napthalenes. Other excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20.

In various embodiments of the therapeutic methods, uses, and compositions disclosed herein, the anti-FcRn antibody, antigen-binding fragment, or pharmaceutical composition can be administered by a variety of methods known in the art. The route and/or mode of administration may vary depending upon the desired results. In some embodiments, the antibody, antigen-binding fragment, or pharmaceutical composition is administered by oral, intravenous, intramuscular, intra-arterial, intramedullary, intradural, intracardial, transdermal, subcutaneous, intraperitoneal, gastrointestinal, sublingual, or local routes. In some embodiments, the antibody, antigen-binding fragment, or pharmaceutical composition is administered orally or parenterally. In some embodiments, the antibody, antigen-binding fragment, or pharmaceutical composition is administered parenterally, e.g., intravenously or subcutaneously (e.g., by injection or infusion). In some embodiments, the antibody, antigen-binding fragment, or pharmaceutical composition is administered subcutaneously (e.g., by injection or infusion). In some embodiments, the antibody, antigen-binding fragment, or pharmaceutical composition is administered as one or more subcutaneous injections. In some embodiments, the antibody, antigen-binding fragment, or pharmaceutical composition is administered as a single (i.e., one) subcutaneous injection. In some embodiments, the antibody, antigen-binding fragment, or pharmaceutical composition is administered as two or more (e.g., two) consecutive subcutaneous injections. In some embodiments, the antibody, antigen-binding fragment, or pharmaceutical composition is administered without intravenous administration (e.g., intravenous induction) prior to the one or more subcutaneous injections. In some embodiments, the antibody, antigen-binding fragment, or pharmaceutical composition is delivered via a syringe, a catheter, a pump delivery system, or a stent. In some embodiments, the antibody, antigen-binding fragment, or pharmaceutical composition is delivered via a syringe (e.g., a pre-filled syringe). Depending on the route of administration, the active compound(s), i.e., the anti-FcRn antibody or antigen-binding fragment, may be coated in a material to protect the compound(s) from the action of acids and other natural conditions that may inactivate the compound(s).

An antibody, antigen-binding fragment, or pharmaceutical composition may be formulated as various forms such as a powder, tablet, capsule, liquid, injection, ointment, or syrup, and/or comprised in a single-dosage or multi-dosage container such as a sealed ampoule, vial, or syringe. In some embodiments, the antibody, antigen-binding fragment, or pharmaceutical composition is formulated as an injectable form. In some embodiments, the antibody, antigen-binding fragment, or pharmaceutical composition is formulated as an aqueous solution, suspension, or emulsion, with one or more excipients, diluents, dispersants, surfactants, binders, and/or lubricants. In some embodiments, the antibody, antigen-binding fragment, or pharmaceutical composition is comprised in a syringe (e.g., a pre-filled syringe). In some embodiments, the antibody, antigen-binding fragment, or pharmaceutical composition is comprised in a syringe having and/or compatible with a small gauge needle (e.g., a needle greater than about 25 gauge, greater than about 26 gauge, greater than about 27 gauge, greater than about 28 gauge, greater than about 29 gauge, and/or greater than about 30 gauge).

In some embodiments, the antibody, antigen-binding fragment, or pharmaceutical composition is formulated to achieve stability and/or to prevent or minimize physical and/or chemical degradation prior to administration. Physical instability may involve processes such as denaturation and aggregation, while common chemical degradation pathways include but are not limited to cross-linking, deamidation, isomerization, oxidation, and fragmentation (see, e.g., Wang et al., J. Pharm. Sci. 91(1):1-26, 2007). As used herein, the term “stable” or “stability” when used to describe an antibody or an antigen-binding fragment thereof means that the antibody or antigen-binding fragment remains intact in a manner to retain activity (e.g., binding to FcRn) and/or achieve a therapeutic effect. In some embodiments, the antibody, antigen-binding fragment, or pharmaceutical composition is formulated with one or more pharmaceutically acceptable carriers (e.g., one or more excipients) such that it is stable under standard storage conditions. In some embodiments, the antibody, antigen-binding fragment, or pharmaceutical composition is formulated with one or more pharmaceutically acceptable carriers (e.g., one or more excipients) such that it is stable at high concentrations. In some embodiments, the antibody or antigen-binding fragment can be stably concentrated at formulations up to about 170 mg/mL or above. In some embodiments, the antibody or antigen-binding fragment can be stably concentrated at formulations above about 170 mg/mL (e.g., at about 180 mg/mL, at about 200 mg/mL, at about 220 mg/mL, or above). In some embodiments, the stably concentrated formulation (e.g., a formulation comprising up to about 170 mg/mL or above of the antibody or antigen-binding fragment) retains acceptable viscosity for administration via a small gauge needle. In some embodiments, the small gauge needle is greater than about 25 gauge, greater than about 26 gauge, greater than about 27 gauge, greater than about 28 gauge, greater than about 29 gauge, or greater than about 30 gauge.

Dosage regimens for the anti-FcRn antibody or antigen-binding fragment, either alone or in combination with one or more additional therapeutic agents, may be adjusted to provide the optimum desired response (e.g., a therapeutic response). For example, a single bolus of the anti-FcRn antibody or antigen-binding fragment may be administered at one time, several divided doses may be administered over a predetermined period of time, or the dose of the anti-FcRn antibody or antigen-binding fragment may be proportionally reduced or increased as indicated by the exigencies of the therapeutic situation. For any particular subject, specific dosage regimens may be adjusted over time according to the individual's need, and the professional judgment of the treating clinician. For instance, in some embodiments, the dose of the anti-FcRn antibody or antigen-binding fragment can be suitably determined by taking into consideration the patient's severity, condition, age, case history, and the like.

The anti-FcRn antibody or antigen-binding fragment may be formulated into pharmaceutically acceptable dosage forms by conventional methods known to those of skill in the art. Parenteral compositions, for example, may be formulated in dosage unit form for ease of administration and uniformity of dosage. As used herein, “dosage unit form” refers to physically discrete units suited as unitary dosages for the subjects to be treated; each unit contains a predetermined quantity of active compound calculated to produce the desired therapeutic effect in association with a pharmaceutically acceptable carrier. In some embodiments, the antibody, antigen-binding fragment, or pharmaceutical composition is formulated in dosage unit form. In some embodiments, the antibody, antigen-binding fragment, or pharmaceutical composition is formulated in dosage unit form for subcutaneous administration. In some embodiments, the antibody, antigen-binding fragment, or pharmaceutical composition is formulated in dosage unit form for administration as one or more subcutaneous injections (e.g., one subcutaneous injection or two or more (e.g., two) consecutive subcutaneous injections). In some embodiments, the antibody, antigen-binding fragment, or pharmaceutical composition is formulated in dosage unit form (e.g., as one or more subcutaneous injections) for self-administration by the patient and/or for administration by a treating clinician.

Dosage values for the anti-FcRn antibody or antigen-binding fragment, compositions comprising the anti-FcRn antibody or antigen-binding fragment, and/or any additional therapeutic agent(s), may be selected based on the unique characteristics of the active compound(s) and the particular therapeutic effect to be achieved. A physician or veterinarian can start doses of the antibodies or antigen-binding fragments at levels lower than those required to achieve the desired therapeutic effect and gradually increase the dosage until the desired effect is achieved. A physician or veterinarian can also start doses of the antibodies or antigen-binding fragments at levels higher than those required to achieve the desired therapeutic effect and gradually decrease the dosage until the desired effect is achieved. In general, effective doses of the antibodies or antigen-binding fragments for the treatment of warm autoimmune hemolytic anemia may vary depending upon many different factors, including whether the treatment is prophylactic or therapeutic. The selected dosage level may also depend upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. In some embodiments, the treatment may be administered once or several times. Intermittent and/or chronic (continuous) dosing strategies may be applied in view of the condition of the particular patient.

In some embodiments, a therapeutically effective amount of the anti-FcRn antibody or antigen-binding fragment is employed in the methods, uses, and pharmaceutical compositions of the present disclosure.

As used herein, the terms “therapeutically effective amount” and “therapeutically effective dose” are used interchangeably herein to refer to an amount sufficient to decrease at least one symptom or measurable parameter associated with a disease, disorder, or condition; to normalize body functions in a disease, disorder, or condition that results in the impairment of specific bodily functions; and/or to provide improvement in, or slow the progression of, one or more clinically measured parameters of a disease, disorder, or condition. A therapeutically effective amount may, for example, be sufficient to treat, prevent, reduce the severity, delay the onset, and/or reduce the risk of occurrence of one or more symptoms of warm autoimmune hemolytic anemia. A therapeutically effective amount, as well as a therapeutically effective frequency of administration, can be determined by methods known in the art and discussed herein. In some embodiments of the methods, uses, and compositions described herein, the anti-FcRn antibody or antigen-binding fragment is administered in an amount that is therapeutically effective when administered as a single agent. In some embodiments, the anti-FcRn antibody or antigen-binding fragment and at least one additional therapeutic agent are each administered in an amount that is therapeutically effective when the agents are used in combination. In some embodiments, a therapeutically effective amount of the anti-FcRn antibody or antigen-binding fragment is the amount required to reduce the level of total serum IgG and/or the level of at least one autoantibody (e.g., at least one IgG) in a patient having or suspected of having warm autoimmune hemolytic anemia. In some embodiments, a therapeutically effective amount of the anti-FcRn antibody or antigen-binding fragment is the amount required to increase the level of hemoglobin in a patient having or suspected of having warm autoimmune hemolytic anemia.

In some embodiments, a therapeutically effective amount of the anti-FcRn antibody or antigen-binding fragment is the amount required to reduce the level of total serum IgG and/or the level of at least one autoantibody and/or pathogenic antibody (e.g., at least one IgG) in a warm autoimmune hemolytic anemia patient and/or in a sample from the patient by at least about 20%, about 25%, about 30%, about 35%, about 40%, about 45%, about 50%, about 55%, about 60%, about 65%, about 70%, about 75%, or about 80% relative to the level prior to treatment with the anti-FcRn antibody or antigen-binding fragment. In some embodiments, a therapeutically effective amount of the anti-FcRn antibody or antigen-binding fragment is the amount required to reduce the level of total serum IgG and/or the level of at least one autoantibody and/or pathogenic antibody (e.g., at least one IgG) in a warm autoimmune hemolytic anemia patient and/or in a sample from the patient by at least about 40%, about 50%, about 60%, about 70%, or about 80% relative to the level prior to treatment with the anti-FcRn antibody or antigen-binding fragment. In some embodiments, a therapeutically effective amount of the anti-FcRn antibody or antigen-binding fragment is the amount required to reduce the serum endogenous IgG concentration in a warm autoimmune hemolytic anemia patient and/or in a sample from the patient to less than about 40%, about 50%, about 60%, about 70%, or about 80% of pretreatment values.

The phrase “total IgG level” or “level of total serum IgG,” as used herein, refers to the serum endogenous IgG concentration, e.g., in a patient or in a biological sample (e.g., a blood sample) from a patient.

The phrase “level of at least one autoantibody,” as used herein, refers to the serum endogenous concentration of the at least one autoantibody, e.g., in a patient or in a biological sample from a patient.

The phrase “level of at least one IgG,” as used herein, refers to the serum endogenous concentration of the at least one IgG, e.g., in a patient or in a biological sample from a patient. In some embodiments, the at least one IgG comprises a pathogenic IgG. In some embodiments, the at least one IgG comprises serum IgG1. In some embodiments, the at least one IgG comprises serum IgG2. In some embodiments, the at least one IgG comprises serum IgG3. In some embodiments, the at least one IgG comprises serum IgG4.

In some embodiments, a therapeutically effective amount of the anti-FcRn antibody or antigen-binding fragment is the amount required to increase the level of hemoglobin in a warm autoimmune hemolytic anemia patient and/or in a sample from the patient by at least about 5%, about 10%, about 15%, or about 20% (e.g., about 5% to about 30%) relative to the level prior to treatment with the anti-FcRn antibody or antigen-binding fragment. In some embodiments, a therapeutically effective amount of the anti-FcRn antibody or antigen-binding fragment is the amount required to increase the level of hemoglobin in a warm autoimmune hemolytic anemia patient and/or in a sample from the patient by at least about 10% (e.g., about 10% to about 15%) after about 1 or 2 weeks of weekly dosing, relative to the level prior to treatment with the anti-FcRn antibody or antigen-binding fragment. In some embodiments, a therapeutically effective amount of the anti-FcRn antibody or antigen-binding fragment is the amount required to increase the level of hemoglobin in a warm autoimmune hemolytic anemia patient and/or in a sample from the patient by at least about 20% (e.g., about 20% to about 25%) after about 1 or 2 weeks of weekly dosing, relative to the level prior to treatment with the anti-FcRn antibody or antigen-binding fragment. In some embodiments, an increase in the level of hemoglobin in a patient and/or a sample from a patient (e.g., an increase of about 10%, about 20%, or more) is maintained during the entire treatment period or a portion thereof. In some embodiments, an increase in the level of hemoglobin in a patient and/or a sample from a patient (e.g., an increase of about 10%, about 20%, or more) is maintained for at least 2, 3, or 4 weeks (e.g., 4 weeks or longer). In some embodiments, an increase in the level of hemoglobin in a patient and/or a sample from a patient (e.g., an increase of about 10%, about 20%, or more) is maintained for about 2 to about 6 weeks.

The term “about” or “approximately,” as used herein in the context of numerical values and ranges, refers to values or ranges that approximate or are close to the recited values or ranges such that the embodiment may perform as intended, as is apparent to the skilled person from the teachings contained herein. These terms encompass values beyond those resulting from systematic error. In some embodiments, “about” or “approximately” means plus or minus 10% of a numerical amount.

In various embodiments of the therapeutic methods and uses disclosed herein, the antibody or antigen-binding fragment is administered to a patient as a fixed dose. In various embodiments of the therapeutic methods and uses disclosed herein, the antibody or antigen-binding fragment is administered to a patient as a weight-based dose, i.e., a dose dependent on the patient's bodyweight. In various embodiments of the therapeutic methods and uses disclosed herein, the antibody or antigen-binding fragment is administered to a patient as a body surface area-based dose, i.e., a dose dependent on the patient's body surface area (BSA). In various embodiments, the dose administered to the patient comprises a therapeutically effective amount of the antibody or antigen-binding fragment.

In some embodiments, the antibody or antigen-binding fragment is administered to the patient at dose of about 170 mg to about 1500 mg. In some embodiments, the antibody or antigen-binding fragment is administered to the patient at dose of about 300 mg to about 800 mg. In some embodiments, the antibody or antigen-binding fragment is administered to the patient at a dose of about 170 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, or about 1500 mg, e.g., once weekly or once every 2 weeks.

In some embodiments, the antibody or antigen-binding fragment is administered to the patient at a dose of about 170 mg to about 300 mg. In some embodiments, the antibody or antigen-binding fragment is administered to the patient at a dose of about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg.

In some embodiments, the antibody or antigen-binding fragment is administered to the patient at a dose of about 300 mg to about 500 mg. In some embodiments, the antibody or antigen-binding fragment is administered to the patient at a dose of about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg.

In some embodiments, the antibody or antigen-binding fragment is administered to the patient at a dose of about 300 mg to about 400 mg. In some embodiments, the antibody or antigen-binding fragment is administered to the patient at a dose of about 300 mg, about 310 mg, about 320 mg, about 330 mg, about 340 mg, about 350 mg, about 360 mg, about 370 mg, about 380 mg, about 390 mg, or about 400 mg. In some embodiments, the antibody or antigen-binding fragment is administered to the patient at a dose of about 320 mg, about 330 mg, about 340 mg, about 350 mg, or about 360 mg. In some embodiments, the antibody or antigen-binding fragment is administered to the patient at a dose of about 340 mg. In some embodiments, the antibody or antigen-binding fragment is administered to the patient at a dose of about 340 mg once weekly or once every 2 weeks. In some embodiments, the antibody or antigen-binding fragment is administered to the patient at a dose of about 340 mg once weekly. In some embodiments, the antibody or antigen-binding fragment is administered to the patient at a dose of about 340 mg once weekly as a single subcutaneous injection. In some embodiments, the antibody or antigen-binding fragment is administered to the patient at a dose of about 340 mg once weekly for at least 2 weeks (e.g., 2 weeks, 3 weeks, 4 weeks, 5 weeks, 6 weeks, 7 weeks, 8 weeks, 10 weeks, 12 weeks, or longer). In some embodiments, the antibody or antigen-binding fragment is administered to the patient at a dose of about 340 mg once weekly for at least 4 weeks. In some embodiments, the antibody or antigen-binding fragment is administered to the patient at a dose of about 340 mg once weekly for at least 7 weeks. In some embodiments, the antibody or antigen-binding fragment is administered to the patient at a dose of about 340 mg once weekly for at least 12 weeks.

In some embodiments, the antibody or antigen-binding fragment is administered to the patient at a dose of about 500 mg to about 700 mg. In some embodiments, the antibody or antigen-binding fragment is administered to the patient at a dose of about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg.

In some embodiments, the antibody or antigen-binding fragment is administered to the patient at a dose of about 650 mg to about 750 mg. In some embodiments, the antibody or antigen-binding fragment is administered to the patient at a dose of about 650 mg, about 660 mg, about 670 mg, about 680 mg, about 690 mg, about 700 mg, about 710 mg, about 720 mg, about 730 mg, about 740 mg, or about 750 mg. In some embodiments, the antibody or antigen-binding fragment is administered to the patient at a dose of about 660 mg, about 670 mg, about 680 mg, about 690 mg, or about 700 mg. In some embodiments, the antibody or antigen-binding fragment is administered to the patient at a dose of about 680 mg. In some embodiments, the antibody or antigen-binding fragment is administered to the patient at a dose of about 680 mg once weekly or once every 2 weeks. In some embodiments, the antibody or antigen-binding fragment is administered to the patient at a dose of about 680 mg once weekly. In some embodiments, the antibody or antigen-binding fragment is administered to the patient at a dose of about 680 mg once weekly as two or more (e.g., two) consecutive subcutaneous injections. In some embodiments, the antibody or antigen-binding fragment is administered to the patient at a dose of about 680 mg once weekly for at least 2 weeks (e.g., 2 weeks, 3 weeks, 4 weeks, 5 weeks, 6 weeks, 7 weeks, 8 weeks, 10 weeks, 12 weeks, or longer). In some embodiments, the antibody or antigen-binding fragment is administered to the patient at a dose of about 680 mg once weekly for at least 4 weeks. In some embodiments, the antibody or antigen-binding fragment is administered to the patient at a dose of about 680 mg once weekly for at least 7 weeks. In some embodiments, the antibody or antigen-binding fragment is administered to the patient at a dose of about 680 mg once weekly for at least 12 weeks.

In some embodiments, the antibody or antigen-binding fragment is administered to the patient at a dose of about 700 mg to about 900 mg. In some embodiments, the antibody or antigen-binding fragment is administered to the patient at a dose of about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, or about 900 mg.

In some embodiments, the antibody or antigen-binding fragment is administered to the patient at a dose of about 900 mg to about 1100 mg. In some embodiments, the antibody or antigen-binding fragment is administered to the patient at a dose of about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, or about 1100 mg.

In some embodiments, the antibody or antigen-binding fragment is administered to the patient at a dose of about 1100 mg to about 1300 mg. In some embodiments, the antibody or antigen-binding fragment is administered to the patient at a dose of about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, or about 1300 mg.

In some embodiments, the antibody or antigen-binding fragment is administered to the patient at a dose of about 1300 mg to about 1500 mg. In some embodiments, the antibody or antigen-binding fragment is administered to the patient at a dose of about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, or about 1500 mg.

In some embodiments, the antibody or antigen-binding fragment is administered to the patient at a dose of about 1 mg/kg to about 2000 mg/kg bodyweight. In some embodiments, the antibody or antigen-binding fragment is administered to the patient at a dose of about 1 mg/kg to about 200 mg/kg, about 200 mg/kg to about 400 mg/kg, about 400 mg/kg to about 600 mg/kg, about 600 mg/kg to about 800 mg/kg, about 800 mg/kg to about 1000 mg/kg, about 1000 mg/kg to about 1200 mg/kg, about 1200 mg/kg to about 1400 mg/kg, about 1400 mg/kg to about 1600 mg/kg, about 1600 mg/kg to about 1800 mg/kg, or about 1800 mg/kg to about 2000 mg/kg. In some embodiments, the antibody or antigen-binding fragment is administered to the patient at a dose of about 1 mg/kg to about 200 mg/kg. In some embodiments, the antibody or antigen-binding fragment is administered to the patient at a dose of about 1 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the antibody or antigen-binding fragment is administered to the patient at a dose of about 1 mg/kg to about 40 mg/kg. In some embodiments, the antibody or antigen-binding fragment is administered to the patient at a dose of about 1 mg/kg, about 5 mg/kg, about 10 mg/kg, about 15 mg/kg, about 20 mg/kg, about 25 mg/kg, about 30 mg/kg, about 35 mg/kg, or about 40 mg/kg.

The frequency with which the antibody or antigen-binding fragment is administered to the patient, as a single agent or in combination with one or more additional therapeutic agents, may be once or more than once. In some embodiments, the antibody or antigen-binding fragment is administered on a single occasion. In some embodiments, the antibody or antigen-binding fragment is administered on multiple occasions. Intervals between dosages can be, e.g., daily, weekly, bi-weekly, monthly, or yearly. Intervals can also be irregular, e.g., based on measuring blood levels of the antibody or antigen-binding fragment in the patient in order to maintain a relatively consistent plasma concentration of the antibody or antigen-binding fragment; based on measuring levels of at least one autoantibody and/or pathogenic antibody (e.g., at least one IgG) in order to maintain a reduced level of the at least one autoantibody and/or pathogenic antibody (e.g., the at least one IgG) so as to provide the desired therapeutic or prophylactic effect; based on measuring levels of total serum IgG in order to maintain a reduced level of total serum IgG so as to provide the desired therapeutic or prophylactic effect; and/or based on measuring levels of hemoglobin in order to maintain an increased level of hemoglobin so as to provide the desired therapeutic or prophylactic effect. Alternatively, in some embodiments, the antibody or antigen-binding fragment can be administered as a sustained release formulation, in which case less frequent administration is required. Dosage and frequency may vary depending on the half-life of the antibody or antigen-binding fragment in the patient. The dosage and frequency of administration may also vary depending on whether the treatment is prophylactic or therapeutic. In prophylactic applications, a relatively low dosage may be administered at relatively infrequent intervals over a long period of time. Some patients may continue to receive treatment for the rest of their lives. In therapeutic applications, a relatively higher dosage at relatively shorter intervals is sometimes administered until progression of the disease is reduced or terminated, and optionally until the patient shows partial or complete amelioration of one or more symptoms of the disease. Thereafter, the patient may be administered a lower, e.g., prophylactic regime.

In some embodiments, the antibody, antigen-binding fragment, or pharmaceutical composition is administered to the patient once or more than once over a period of about 1 day, 2 days, 3 days, 4 days, 5 days, 6 days, 7 days, 8 days, 9 days, 10 days, 11 days, 12 days, 13 days, 14 days, 1 week, 2 weeks, 3 weeks, 4 weeks, 1 month, 2 months, 3 months, 4 months, 5 months, 6 months, 7 months, 8 months, 9 months, 10 months, 11 months, 12 months, 18 months, 24 months, 30 months, 36 months, or longer.

In some embodiments, the antibody, antigen-binding fragment, or pharmaceutical composition is administered to the patient once as a single dose.

In some embodiments, the antibody, antigen-binding fragment, or pharmaceutical composition is administered to the patient once weekly. In some embodiments, the antibody, antigen-binding fragment, or pharmaceutical composition is administered to the patient once weekly for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 12 weeks, at least 20 weeks, at least 24 weeks, at least 30 weeks, at least 40 weeks, at least 50 weeks, at least 60 weeks, at least 70 weeks, at least 76 weeks, at least 80 weeks, or longer. In some embodiments, the antibody, antigen-binding fragment, or pharmaceutical composition is administered to the patient once weekly for 6 to 76 weeks, or any time period in between. In some embodiments, the antibody, antigen-binding fragment, or pharmaceutical composition is administered to the patient once weekly for at least 2 weeks, at least 3 weeks, at least 4 weeks, or at least 6 weeks. In some embodiments, the antibody, antigen-binding fragment, or pharmaceutical composition is administered to the patient once weekly for at least 4 weeks. In some embodiments, the antibody, antigen-binding fragment, or pharmaceutical composition is administered to the patient once weekly for at least 7 weeks. In some embodiments, the antibody, antigen-binding fragment, or pharmaceutical composition is administered to the patient once weekly for at least 12 weeks. In some embodiments, the antibody, antigen-binding fragment, or pharmaceutical composition is administered to the patient once weekly until sufficient to treat, prevent, reduce the severity, delay the onset, and/or reduce the risk of occurrence of one or more symptoms of warm autoimmune hemolytic anemia (e.g., pallor, fatigue, jaundice, enlargement of the spleen).

In some embodiments, the antibody, antigen-binding fragment, or pharmaceutical composition is administered to the patient once weekly as a single (i.e., one) subcutaneous injection. In some embodiments, the antibody, antigen-binding fragment, or pharmaceutical composition is administered to the patient once weekly as two or more consecutive subcutaneous injections (e.g., two consecutive subcutaneous injections). The term “consecutive,” as used herein in the context of subcutaneous injections (or other routes of administration), refers to two or more subcutaneous injections administered one after another, but sufficiently close in time so as to provide the desired therapeutic or prophylactic effect. In some embodiments, consecutive subcutaneous injections are administered within about 30 seconds, within about 1 minute, within about 2 minutes, within about 5 minutes, within about 10 minutes, within about 30 minutes, within about 1 hour, within about 2 hours, or within about 5 hours of one another.

In some embodiments, the antibody, antigen-binding fragment, or pharmaceutical composition is administered to the patient once every 2 weeks (bi-weekly). In some embodiments, the antibody, antigen-binding fragment, or pharmaceutical composition is administered to the patient once every 2 weeks for at least 2 weeks, at least 4 weeks, at least 6 weeks, at least 8 weeks, at least 10 weeks, at least 12 weeks, at least 20 weeks, at least 24 weeks, at least 30 weeks, at least 40 weeks, at least 50 weeks, at least 60 weeks, at least 70 weeks, at least 76 weeks, at least 80 weeks, or longer. In some embodiments, the antibody, antigen-binding fragment, or pharmaceutical composition is administered to the patient once every 2 weeks for 6 to 76 weeks, or any time period in between. In some embodiments, the antibody, antigen-binding fragment, or pharmaceutical composition is administered to the patient once every 2 weeks for at least 12 weeks. In some embodiments, the antibody, antigen-binding fragment, or pharmaceutical composition is administered to the patient once every 2 weeks until sufficient to treat, prevent, reduce the severity, delay the onset, and/or reduce the risk of occurrence of one or more symptoms of warm autoimmune hemolytic anemia. In some embodiments, the antibody, antigen-binding fragment, or pharmaceutical composition is administered to the patient once every 2 weeks as a single subcutaneous injection. In some embodiments, the antibody, antigen-binding fragment, or pharmaceutical composition is administered to the patient once every 2 weeks as two or more consecutive subcutaneous injections.

In some embodiments, the antibody, antigen-binding fragment, or pharmaceutical composition is administered to the patient once monthly. In some embodiments, the antibody, antigen-binding fragment, or pharmaceutical composition is administered to the patient once monthly for at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or longer. In some embodiments, the antibody, antigen-binding fragment, or pharmaceutical composition is administered to the patient once monthly until sufficient to treat, prevent, reduce the severity, delay the onset, and/or reduce the risk of occurrence of one or more symptoms of warm autoimmune hemolytic anemia. In some embodiments, the antibody, antigen-binding fragment, or pharmaceutical composition is administered to the patient once monthly as a single subcutaneous injection. In some embodiments, the antibody, antigen-binding fragment, or pharmaceutical composition is administered to the patient once monthly as two or more consecutive subcutaneous injections.

In some embodiments of the therapeutic methods, uses, and compositions disclosed herein, the therapeutically effective amount of the antibody or antigen-binding fragment is about 170 mg to about 1500 mg administered once as a single dose. More specifically, in some embodiments, the therapeutically effective amount of the antibody or antigen-binding fragment is about 170 mg to about 300 mg administered once as a single dose. In some embodiments, the therapeutically effective amount of the antibody or antigen-binding fragment is about 300 mg to about 500 mg administered once as a single dose. In some embodiments, the therapeutically effective amount of the antibody or antigen-binding fragment is about 500 mg to about 700 mg administered once as a single dose. In some embodiments, the therapeutically effective amount of the antibody or antigen-binding fragment is about 700 mg to about 900 mg administered once as a single dose. In some embodiments, the therapeutically effective amount of the antibody or antigen-binding fragment is about 900 mg to about 1100 mg administered once as a single dose. In some embodiments, the therapeutically effective amount of the antibody or antigen-binding fragment is about 1100 mg to about 1300 mg administered once as a single dose. In some embodiments, the therapeutically effective amount of the antibody or antigen-binding fragment is about 1300 mg to about 1500 mg administered once as a single dose.

In some embodiments of the therapeutic methods, uses, and compositions disclosed herein, the therapeutically effective amount of the antibody or antigen-binding fragment is about 300 mg to about 800 mg administered once as a single dose. In some embodiments, the therapeutically effective amount of the antibody or antigen-binding fragment is about 300 mg to about 400 mg administered once as a single dose. In some embodiments, the therapeutically effective amount of the antibody or antigen-binding fragment is about 340 mg administered once as a single dose. In some embodiments, the therapeutically effective amount of the antibody or antigen-binding fragment is about 450 mg to about 550 mg administered once as a single dose. In some embodiments, the therapeutically effective amount of the antibody or antigen-binding fragment is about 500 mg administered once as a single dose. In some embodiments, the therapeutically effective amount of the antibody or antigen-binding fragment is about 700 mg to about 800 mg administered once as a single dose. In some embodiments, the therapeutically effective amount of the antibody or antigen-binding fragment is about 765 mg administered once as a single dose. In some embodiments, treatment reduces the level of total serum IgG in a patient by at least about 20%, about 25%, about 30%, about 35%, about 40%, about 45%, or about 50%. In some embodiments, treatment reduces the level of total serum IgG in a patient by at least about 25%. In some embodiments, treatment reduces the level of total serum IgG in a patient by at least about 35%. In some embodiments, treatment reduces the level of total serum IgG in a patient by at least about 45%. In some embodiments, maximum reduction in the level of total serum IgG in a patient occurs at about 5 days to about 20 days after administration of the antibody or antigen-binding fragment, or a pharmaceutical composition comprising the antibody or antigen-binding fragment. In some embodiments, maximum reduction in the level of total serum IgG in a patient occurs at about 8 days to about 15 days after administration of the antibody or antigen-binding fragment, or a pharmaceutical composition comprising the antibody or antigen-binding fragment. In some embodiments, maximum reduction in the level of total serum IgG occurs after about 3 to 5 doses (e.g., after about 4 doses) of the antibody or antigen-binding fragment, or a pharmaceutical composition comprising the antibody or antigen-binding fragment. In some embodiments, treatment increases the level of hemoglobin in a patient by at least about 5%, about 10%, about 15%, or about 20% (e.g., about 5% to about 30%). In some embodiments, treatment increases the level of hemoglobin in a patient by at least about 10% (e.g., about 10% to about 15%). In some embodiments, treatment increases the level of hemoglobin in a patient by at least about 20% (e.g., about 20% to about 25%). In some embodiments, treatment increases the level of hemoglobin in a patient by more than about 20% (e.g., about 25%, about 30%, or more).

In some embodiments of the therapeutic methods, uses, and compositions disclosed herein, the therapeutically effective amount of the antibody or antigen-binding fragment is about 170 mg to about 1500 mg administered once weekly. In some embodiments, the therapeutically effective amount of the antibody or antigen-binding fragment is about 170 mg to about 300 mg administered once weekly. In some embodiments, the therapeutically effective amount of the antibody or antigen-binding fragment is about 300 mg to about 500 mg administered once weekly. In some embodiments, the therapeutically effective amount of the antibody or antigen-binding fragment is about 500 mg to about 700 mg administered once weekly. In some embodiments, the therapeutically effective amount of the antibody or antigen-binding fragment is about 700 mg to about 900 mg administered once weekly. In some embodiments, the therapeutically effective amount of the antibody or antigen-binding fragment is about 900 mg to about 1100 mg administered once weekly. In some embodiments, the therapeutically effective amount of the antibody or antigen-binding fragment is about 1100 mg to about 1300 mg administered once weekly. In some embodiments, the therapeutically effective amount of the antibody or antigen-binding fragment is about 1300 mg to about 1500 mg administered once weekly.

In some embodiments of the therapeutic methods, uses, and compositions disclosed herein, the therapeutically effective amount of the antibody or antigen-binding fragment is about 300 mg to about 800 mg administered once weekly. In some embodiments, the therapeutically effective amount of the antibody or antigen-binding fragment is about 300 mg to about 400 mg administered once weekly. In some embodiments, the therapeutically effective amount of the antibody or antigen-binding fragment is about 340 mg administered once weekly. In some embodiments, the therapeutically effective amount of the antibody or antigen-binding fragment is about 650 mg to about 750 mg administered once weekly. In some embodiments, the therapeutically effective amount of the antibody or antigen-binding fragment is about 680 mg administered once weekly. In some embodiments, treatment reduces the level of total serum IgG in a patient by at least about 50%, about 55%, about 60%, about 65%, about 70%, about 75%, or about 80%. In some embodiments, treatment reduces the level of total serum IgG in a patient by at least about 60%. In some embodiments, treatment reduces the level of total serum IgG in a patient by at least about 70%. In some embodiments, treatment reduces the level of total serum IgG in a patient by at least about 80%. In some embodiments, maximum reduction in the level of total serum IgG in a patient occurs at about 20 days to about 30 days after administration of the antibody or antigen-binding fragment, or a pharmaceutical composition comprising the antibody or antigen-binding fragment. In some embodiments, maximum reduction in the level of total serum IgG in a patient occurs at about 24 days after administration of the antibody or antigen-binding fragment, or a pharmaceutical composition comprising the antibody or antigen-binding fragment. In some embodiments, maximum reduction in the level of total serum IgG occurs after about 3 to 5 doses (e.g., after about 4 doses) of the antibody or antigen-binding fragment, or a pharmaceutical composition comprising the antibody or antigen-binding fragment. In some embodiments, treatment increases the level of hemoglobin in a patient by at least about 5%, about 10%, about 15%, or about 20% (e.g., about 5% to about 30%). In some embodiments, treatment increases the level of hemoglobin in a patient by more than about 20%. In some embodiments, treatment increases the level of hemoglobin in a patient by at least about 10% (e.g., about 10% to about 15%) after about 1 or 2 weeks of weekly dosing (e.g., 680 mg administered once weekly). In some embodiments, treatment increases the level of hemoglobin in a patient by at least about 20% (e.g., about 20% to about 25%) after about 1 or 2 weeks of weekly dosing (e.g., 680 mg administered once weekly). In some embodiments, an increase in the level of hemoglobin in a patient (e.g., an increase of about 10%, about 20%, or more) is maintained during the entire treatment period or a portion thereof. In some embodiments, an increase in the level of hemoglobin in a patient (e.g., an increase of about 10%, about 20%, or more) is maintained for at least 4 weeks (e.g., at least 4 weeks, 6 weeks, 8 weeks, 10 weeks, 12 weeks, or longer).

In some embodiments of the therapeutic methods, uses, and compositions disclosed herein, the therapeutically effective amount of the antibody or antigen-binding fragment is about 170 mg to about 1500 mg administered once every 2 weeks. In some embodiments, the therapeutically effective amount of the antibody or antigen-binding fragment is about 300 mg to about 800 mg administered once every 2 weeks. In some embodiments, the therapeutically effective amount of the antibody or antigen-binding fragment is about 170 mg to about 300 mg administered once every 2 weeks. In some embodiments, the therapeutically effective amount of the antibody or antigen-binding fragment is about 300 mg to about 500 mg administered once every 2 weeks. In some embodiments, the therapeutically effective amount of the antibody or antigen-binding fragment is about 500 mg to about 700 mg administered once every 2 weeks. In some embodiments, the therapeutically effective amount of the antibody or antigen-binding fragment is about 700 mg to about 900 mg administered once every 2 weeks. In some embodiments, the therapeutically effective amount of the antibody or antigen-binding fragment is about 900 mg to about 1100 mg administered once every 2 weeks. In some embodiments, the therapeutically effective amount of the antibody or antigen-binding fragment is about 1100 mg to about 1300 mg administered once every 2 weeks. In some embodiments, the therapeutically effective amount of the antibody or antigen-binding fragment is about 1300 mg to about 1500 mg administered once every 2 weeks.

In some embodiments of the therapeutic methods, uses, and compositions disclosed herein, the therapeutically effective amount of the antibody or antigen-binding fragment is about 170 mg to about 1500 mg administered once monthly. In some embodiments, the therapeutically effective amount of the antibody or antigen-binding fragment is about 300 mg to about 800 mg administered once monthly. In some embodiments, the therapeutically effective amount of the antibody or antigen-binding fragment is about 170 mg to about 300 mg administered once monthly. In some embodiments, the therapeutically effective amount of the antibody or antigen-binding fragment is about 300 mg to about 500 mg administered once monthly. In some embodiments, the therapeutically effective amount of the antibody or antigen-binding fragment is about 500 mg to about 700 mg administered once monthly. In some embodiments, the therapeutically effective amount of the antibody or antigen-binding fragment is about 700 mg to about 900 mg administered once monthly. In some embodiments, the therapeutically effective amount of the antibody or antigen-binding fragment is about 900 mg to about 1100 mg administered once monthly. In some embodiments, the therapeutically effective amount of the antibody or antigen-binding fragment is about 1100 mg to about 1300 mg administered once monthly. In some embodiments, the therapeutically effective amount of the antibody or antigen-binding fragment is about 1300 mg to about 1500 mg administered once monthly.

In some embodiments of the therapeutic methods, uses, and compositions disclosed herein, the therapeutically effective amount of an antibody or antigen-binding fragment is about 340 mg or about 680 mg (e.g., about 680 mg) administered once weekly.

In some embodiments, treatment with the antibody or antigen-binding fragment at a dose of about 340 mg or about 680 mg (e.g., about 680 mg) administered once weekly reduces the level of total serum IgG in a patient and/or a sample from a patient by at least about 40% (e.g., about 40% to about 50%) after about 1 or 2 weeks of weekly dosing, relative to the level of total serum IgG in the patient and/or sample prior to treatment. In some embodiments, treatment with the antibody or antigen-binding fragment at a dose of about 340 mg or about 680 mg (e.g., about 680 mg) administered once weekly reduces the level of total serum IgG in a patient and/or a sample from a patient by at least about 60% (e.g., about 60% to about 70%) after about 3 weeks of weekly dosing, relative to the level of total serum IgG in the patient and/or sample prior to treatment. In some embodiments, treatment with the antibody or antigen-binding fragment at a dose of about 340 mg or about 680 mg (e.g., about 680 mg) administered once weekly reduces the level of total serum IgG in a patient and/or a sample from a patient by at least about 70% (e.g., about 70% to about 80%) after about 5 weeks of weekly dosing, relative to the level of total serum IgG in the patient and/or sample prior to treatment. In some embodiments, the therapeutically effective amount of the antibody or antigen-binding fragment is about 680 mg administered once weekly. In some embodiments, the therapeutically effective amount of the antibody or antigen-binding fragment is about 680 mg administered once weekly for at least 2 weeks (e.g., 2 weeks, 3 weeks, 4 weeks, 5 weeks, 6 weeks, 7 weeks, 8 weeks, 10 weeks, 12 weeks, or longer, e.g., 4 weeks, 7 weeks, 12 weeks, or longer).

In some embodiments, treatment with the antibody or antigen-binding fragment at a dose of about 340 mg or about 680 mg (e.g., about 680 mg) administered once weekly increases the level of hemoglobin in a patient and/or a sample from a patient by at least about 10% (e.g., about 10% to about 15%) after about 1 or 2 weeks of weekly dosing, relative to the level of hemoglobin in the patient and/or sample prior to treatment. In some embodiments, treatment with the antibody or antigen-binding fragment at a dose of about 340 mg or about 680 mg (e.g., about 680 mg) administered once weekly increases the level of hemoglobin in a patient and/or a sample from a patient by at least about 20% (e.g., about 20% to about 25%) after about 1 or 2 weeks of weekly dosing, relative to the level of hemoglobin in the patient and/or sample prior to treatment. In some embodiments, an increase in the level of hemoglobin in a patient and/or a sample from a patient (e.g., an increase of about 10%, about 20%, or more) is maintained during the entire treatment period or a portion thereof. In some embodiments, an increase in the level of hemoglobin in a patient and/or a sample from a patient (e.g., an increase of about 10%, about 20%, or more) is maintained for at least 2, 3, or 4 weeks (e.g., 4 weeks or longer). In some embodiments, an increase in the level of hemoglobin in a patient and/or a sample from a patient (e.g., an increase of about 10%, about 20%, or more) is maintained for about 2 to about 6 weeks. In some embodiments, the therapeutically effective amount of the antibody or antigen-binding fragment is about 680 mg administered once weekly for at least 2 weeks (e.g., 2 weeks, 3 weeks, 4 weeks, 5 weeks, 6 weeks, 7 weeks, 8 weeks, 10 weeks, 12 weeks, or longer, e.g., 4 weeks, 7 weeks, 12 weeks, or longer).

In various embodiments, the present disclosure also provides a kit for use in the therapeutic applications described herein. In various embodiments, the present disclosure provides a kit comprising the anti-FcRn antibody or an antigen-binding fragment thereof for use in the treatment of prevention of warm autoimmune hemolytic anemia. In various embodiments, the kit further comprises one or more additional components, including but not limited to: instructions for use; other agents, e.g., one or more additional therapeutic agents (e.g., one or more standard-of-care agents); devices, containers, or other materials for preparing the antibody or antigen-binding fragment for therapeutic administration; pharmaceutically acceptable carriers (e.g., excipients); and devices, containers, or other materials for administering the antibody or antigen-binding fragment to a patient. Instructions for use can include guidance for therapeutic applications including suggested dosages and/or modes of administration, e.g., in a patient having or suspected of having warm autoimmune hemolytic anemia. In various embodiments, the kit comprises the anti-FcRn antibody or an antigen-binding fragment thereof and instructions for therapeutic use, e.g., the use of the antibody or antigen-binding fragment to treat or prevent warm autoimmune hemolytic anemia in a patient. In various embodiments, the kit further contains at least one additional therapeutic agent (e.g., for administering in combination with the antibody or antigen-binding fragment). In various embodiments, the antibody or antigen-binding fragment is formulated as a pharmaceutical composition.

In some embodiments, the anti-FcRn antibody or antigen-binding fragment is produced by expression and purification using a gene recombination method. In some embodiments, polynucleotide sequences that encode the variable regions of the antibody or antigen-binding fragment are produced by expression in separate host cells or simultaneously in a single host cell.

As used herein, the term “recombinant vector” refers to an expression vector capable of expressing a protein of interest in a suitable host cell. The term encompasses a DNA construct including essential regulatory elements operably linked to express a nucleic acid insert.

As used herein, the term “operably linked” refers to a nucleic acid expression control sequence functionally linked to a nucleic acid sequence encoding a protein of interest so as to execute general functions. Operable linkage with the recombinant vector can be performed using a gene recombination technique well known in the art, and site-specific DNA cleavage and ligation can be easily performed using enzymes generally known in the art.

A suitable expression vector may include expression regulatory elements such as a promoter, an operator, an initiation codon, a stop codon, a polyadenylation signal, and an enhancer, as well as a signal sequence for membrane targeting or secretion. The initiation and stop codons are generally considered as part of a nucleotide sequence encoding the immunogenic target protein, and are necessary to be functional in an individual to whom a genetic construct has been administered, and must be in frame with the coding sequence. Promoters may generally be constitutive or inducible. Prokaryotic promoters include, but are not limited to, lac, tac, T3 and T7 promoters. Eukaryotic promoters include, but are not limited to, simian virus 40 (SV40) promoter, mouse mammary tumor virus (MMTV) promoter, human immunodeficiency virus (HIV) promoter such as the HIV Long Terminal Repeat (LTR) promoter, moloney virus promoter, cytomegalovirus (CMV) promoter, epstein barr virus (EBV) promoter, rous sarcoma virus (RSV) promoter, as well as promoters from human genes such as human β-actin, human hemoglobin, human muscle creatine, and human metallothionein. The expression vector may include a selectable marker that allows selection of host cells containing the vector. Genes coding for products that confer selectable phenotypes, such as resistance to drugs, a nutrient requirement, or resistance to cytotoxic agents or expression of surface proteins, may be used as general selectable markers. Since only cells expressing a selectable marker survive in the environment treated with a selective agent, transformed cells can be selected. Also, a replicable expression vector may include a replication origin, a specific nucleic acid sequence that initiates replication. Recombinant expression vectors that may be used include various vectors such as plasmids, viruses, and cosmids. The kind of recombinant vector is not limited, and the recombinant vector could function to express a desired gene and produce a desired protein in various host cells such as prokaryotic and eukaryotic cells. In some embodiments, a vector that can produce a large amount of a foreign protein similar to a natural protein while having strong expression ability with a promoter showing strong activity is used.

A variety of expression host/vector combinations may be used to express the anti-FcRn antibody or an antigen-binding fragment thereof. For example, expression vectors suitable for the eukaryotic host include, but are not limited to, SV40, bovine papillomavirus, adenovirus, adeno-associated virus, cytomegalovirus, and retrovirus. Expression vectors that may be used for bacterial hosts include bacterial plasmids such as pET, pRSET, pBluescript, pGEX2T, pUC, col E1, pCR1, pBR322, pMB9 and derivatives thereof, a plasmid such as RP4 having a wider host range, phage DNA represented as various phage lambda derivatives such as gt10, gt11 and NM989, and other DNAphages such as M13 and filamentous single-stranded DNA phage. Expression vectors useful in yeast cells include 2 μm plasmid and derivatives thereof. A vector useful in insect cells is pVL941.

In some embodiments, the recombinant vector is introduced into a host cell to form a transformant. Host cells suitable for use include prokaryotic cells such as E. coli, Bacillus subtilis, Streptomyces sp., Pseudomonas sp., Proteus mirabilis and Staphylococcus sp., fungi such as Aspergillus sp., yeasts such as Pichia pastoris, Saccharomyces cerevisiae, Schizosaccharomyces sp., and Neurospora crassa, and eukaryotic cells such as lower eukaryotic cells, and higher other eukaryotic cells such as insect cells.

In some embodiments, host cells are derived from plants or animals (e.g., mammals), and examples thereof include, but are not limited to, monkey kidney cells (COS7), NSO cells, SP2/0, Chinese hamster ovary (CHO) cells, W138, baby hamster kidney (BHK) cells, MDCK, myeloma cells, HuT 78 cells and HEK293 cells. In some embodiments, CHO cells are used.

Transfection or transformation into a host cell may include any method by which nucleic acids can be introduced into organisms, cells, tissues or organs, and, as known in the art, may be performed using a suitable standard technique selected according to the kind of host cell. Methods include, but are not limited to, electroporation, protoplast fusion, calcium phosphate (CaPO₄) precipitation, calcium chloride (CaCl₂)) precipitation, agitation with silicon carbide fiber, and Agrobacterium-, PEG-, dextran sulfate-, lipofectamine- and desiccation/inhibition-mediated transformation.

The anti-FcRn antibody or antigen-binding fragment can be produced in large amounts by culturing the transformant comprising the recombinant vector in nutrient medium, and the medium and culture conditions that are used can be selected depending on the kind of host cell. During culture, conditions, including temperature, the pH of medium, and culture time, can be controlled so as to be suitable for the growth of cells and the mass production of protein. The antibody or antigen-binding fragment produced by the recombination method as described herein can be collected from the medium or cell lysate and can be isolated and purified by conventional biochemical isolation techniques (Sambrook et al., Molecular Cloning: A Laboratory Manual, 2nd Ed., Cold Spring Harbor Laboratory Press (1989); Deuscher, Guide to Protein Purification Methods Enzymology, Vol. 182. Academic Press. Inc., San Diego, Calif. (1990)). These techniques include, but are not limited to, electrophoresis, centrifugation, gel filtration, precipitation, dialysis, chromatography (e.g., ion exchange chromatography, affinity chromatography, immunosorbent chromatography, size exclusion chromatography, etc.), isoelectric point focusing, and various modifications and combinations thereof. In some embodiments, the antibody or antigen-binding fragment is isolated and purified using protein A.

### EXAMPLES

Hereinafter, the present disclosure will be described in further detail with reference to examples. It will be obvious to a person having ordinary skill in the art that these examples are illustrative purposes only and are not to be construed to limit the scope of the present disclosure.

### Example 1: Construction of Anti-FcRn-Expressing Library Using Transgenic Rats

Immunization was performed using a total of six transgenic rats (OmniRat®, OMT). As an immunogen, human FcRn was used. Both footpads of the rats were immunized eight times with 0.0075 mg of human FcRn (each time) together with an adjuvant at 3-day intervals for 24 days. On Day 28, the rats were immunized with 5-10 μg of the immunogen diluted in PBS buffer. On Day 28, rat serum was collected and used to measure the antibody titer. On Day 31, the rats were euthanized, and the popliteal lymph node and the inguinal lymph node were recovered for fusion with P3X63/AG8.653 myeloma cells.

ELISA analysis was performed to measure the antibody titer in rat serum. Specifically, human FcRn was diluted in PBS (pH 6.0 or pH 7.4) buffer to make 2 μg/mL of a solution, and 100 μL of the solution was coated on each well of a 96-well plate, and then incubated at 4° C. for at least 18 hours. Each well was washed three times with 300 μL of washing buffer (0.05% Tween 20 in PBS) to remove unbound human FcRn, and then 200 μL of blocking buffer was added to each well and incubated at room temperature for 2 hours. A test serum sample was diluted at 1/100, and then the solution was serially 2-fold diluted to make a total of 10 test samples having a dilution factor of 1/100 to 1/256,000). After blocking, each well was washed with 300 μL of washing buffer, and then each test sample was added to each cell and incubated at room temperature for 2 hours. After washing three times, 100 μL of a 1:50,000 dilution of secondary detection antibody in PBS buffer was added to each well and incubated at room temperature for 2 hours. After washing three times again, 100 μL of TMB solution was added to each well and allowed to react at room temperature for 10 minutes, and then 50 μL of 1 M sulfuric acid-containing stop solution was added to each well to stop the reaction, after which the OD value at 450 nm was measured with a microplate reader. The anti-human FcRn (hFcRn) IgG titer resulting from immunization was higher than that in the pre-immune serum of the rats.

A total of three hybridoma libraries A, B and C fused using polyethylene glycol were made. Specifically, transgenic rats 1 and 5 were used to make hybridoma library A, and rats 2 and 6 were used to make hybridoma library B, and rats 3 and 4 were used to make hybridoma library C. A hybridoma library fusion mixture for constructing each hybridoma library was cultured in HAT-containing medium for 7 days so that only cells fused to HAT would be selected. Hybridoma cells viable in the HAT medium were collected and cultured in HT media for about 6 days, and then the supernatant was collected, and the amount of rat IgG in the supernatant was measured using a rat IgG ELISA kit (RD-biotech). Specifically, each sample was diluted at 1:100, and 100 μL of the dilution was added to each well of an ELISA plate and mixed with peroxidase-conjugated anti-rat IgG, followed by reaction at room temperature for 15 minutes. 100 μL of TMB solution was added to each well and allowed to react at room temperature for 10 minutes, and then 50 μL of 1 M sulfuric acid-containing stop solution was added to each well to stop the reaction. Next, the OD value at 450 nm was measured with a microplate reader.

### Example 2: Evaluation of the Antigen Binding Affinity and IgG Binding Blocking Ability of Anti-hFcRn Antibodies of Hybridoma Libraries

To analyze the binding of antibodies to hFcRn, the same ELISA analysis (pH 6.0 and pH 7.4) as mentioned above was performed.

Using the culture supernatants of the three hybridoma libraries, the evaluation of the hFcRn binding affinity by FACS at 5 ng/mL and 25 ng/mL was performed at pH 6.0 and pH 7.4. Human FcRn-stable expressing HEK293 cells were detached from a flask, and then suspended in reaction buffer (0.05% BSA in PBS, pH 6.0 or pH 7.4). The suspension was diluted to a cell density of 2×10⁶ cells/mL, and 50 μL of the dilution was added to each well of a 96-well plate. Then, 50 μL of the hybridoma library culture supernatant diluted to each of 10 ng/mL and 50 ng/mL was added to each well and suspended to allow antibody to bind. A488 rabbit anti-IgG goat antibody was diluted at 1:200 in reaction buffer, and 100 μL of the dilution was added to each well and mixed with the cell pellets to perform a binding reaction, and then 150 μL of reaction buffer was added to each well. Measurement was performed in FACS (BD).

Evaluation of the human FcRn blocking ability of the hybridoma library by FACS was performed at pH 6.0. Specifically, naïve HEK293 cells and human FcRn-overexpressing HEK293 cells were suspended in reaction buffer (0.05% BSA in PBS, pH 6.0). 1×10⁵ cells were added to a 96-well plate, and treated with each of 4 nM of each hybridoma library culture supernatant and 0.4 nM of a 10-fold dilution of the supernatant. To confirm the hIgG blocking ability, 100 nM A488-hIgG1 was added to each well, and then incubated on ice for 90 minutes. After completion of the reaction, the cell pellets were washed with 100 μL of reaction buffer, and transferred into a U-shaped round bottom tube, followed by measurement in FACS. The amount of 100 nM A488-hIgG1 remaining in the human FcRn-overexpressing stable cells was measured, and then the blocking (%) was calculated. As an isotype control, hIgG1 was used, and as a positive control, previously developed HL161-1Ag antibody was used to comparatively evaluate the antibody blocking effect. Each control was analyzed at concentrations of 1 μM and 2 μM, and the hybridoma library sample was measured at two concentrations of 0.4 nM and 4 nM.

### Example 3: Isolation of Hybridoma Clone by FACS and Selection of Human Antibodies

Using hybridoma library A showing the highest human FcRn binding affinity and blocking effect, clones were isolated by FACS (flow cytometry) to thereby obtain a total of 442 single clones. The isolated monoclones were cultured in HT media, and the supernatant was collected. Antibody-expressing hybridoma clones binding to hFcRn in the supernatant were selected by FACS.

RNA was isolated from 100 monoclones selected by FACS analysis and the isolated RNA was sequenced. In the first-step sequencing, 88 of the 100 monoclones were sequenced, and divided according to the amino acid sequence into a total of 35 groups (G1 to G38). The culture supernatants of the representative clones of 33 groups excluding two clones (G33 and G35) whose media were not available were diluted at a concentration of 100 ng/mL, and the binding affinity for hFcRn was evaluated by ELISA.

In the same manner as described above, evaluation of the hFcRn binding affinity by FACS was performed at pH 6.0 and 7.4. The order of the binding affinity of the clones was similar between the pHs, and the binding intensity appeared at various levels.

In addition, evaluation of the hFcRn blocking effects of the 33 clones was performed by FACS at pH 6.0. The blocking (%) was calculated based on the measured MFI value. Based on the results of analysis of the blocking % at a concentration of 1667 pM, the clones were divided into a total of the following four groups: group A: 70-100%; group B: 30-70%; group C: 10-30%; and group D: 10% or less.

For kinetic analysis of the hybridoma clones by SPR, human FcRn was immobilized, and then the analysis was performed using the hybridoma culture as an analyte.

Among the five hybridoma clones, the genes of 18 clones having no N-glycosylation site or free cysteine in the CDR sequences of groups A and B divided according to the results of analysis of the hFcRn blocking effect were converted to whole human IgG sequences.

Specifically, the amino acid sequence similarity between the VH and VL of the 18 selected antibodies and the human germ line antibody group was examined using the Ig BLAST program of the NCBI webpage.

In order to clone the 18 human antibody genes, restriction enzyme recognition sites were inserted into both ends of the genes in the following manner. EcoRI/ApaI were inserted into the heavy chain variable domain (VH); EcoRI/XhoI were inserted into the light chain lambda variable domain (VL(λ)); EcoRI/NheI restriction enzyme recognitions sites were inserted into the light chain kappa variable domain (VL(κ)). In the case of the light chain variable domain, the light chain lambda variable (VL(λ)) gene sequence was linked to the human light chain constant (LC(λ)) region gene during gene cloning, and the light chain kappa variable (VL(κ)) gene sequence was linked to the human light chain constant (LC(κ)) region gene.

In cloning into pCHO1.0 expression vectors for expression of antibodies in animal cells, the light chain and heavy chain genes were inserted after cleavage with EcoRV, PacI, AvrII and BstZ17I restriction enzymes. In order to examine whether pCHO1.0 expression vectors containing the 18 selected human antibody genes were consistent with the synthesized gene sequences, DNA sequencing was performed.

Using the pCHO1.0 expression vectors that are animal cell expression systems containing all the antibody light chain and heavy chain genes, whole human IgG was expressed. The human antibody was obtained by transiently transfecting the plasmid DNA of each of the antibodies into CHO-S cells and purifying the antibody, secreted into the medium, by protein A column.

Human IgG was injected into hFcRn-expressing Tg32 (hFcRn+/+, hβ2m+/+, mFcRn−/−, mβ2m−/−) mice (Jackson Laboratory), and then the 18 human antibodies converted to the human IgG sequences were administered to the mice in order to examine whether the antibodies would influence the catabolism of human IgG.

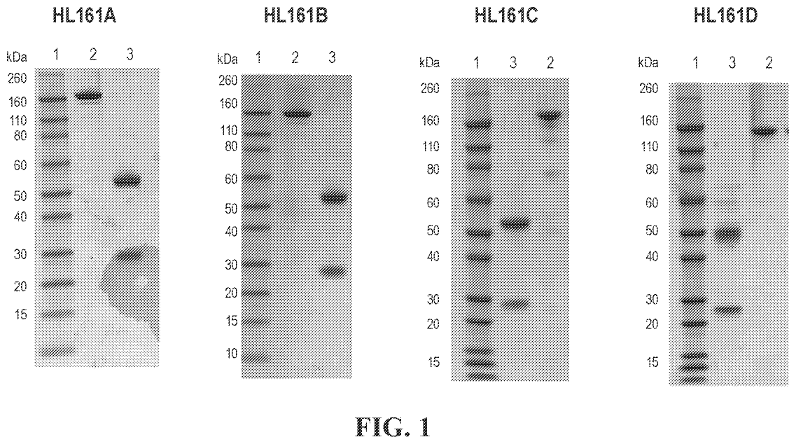

Based on the in vitro analysis results for binding affinity (K_{D}) for the antigen and the analysis of human FcRn binding affinity and blocking effect by FACS, and the in vivo analysis of catabolism of human IgG, four human anti-FcRn antibody proteins (HL161A, HL161B, HL161C and HL161D) were selected (FIG. 1). In addition, an HL161BK antibody having no N-glycosylation site was prepared by substituting the asparagine (N) at position 83 of the heavy chain variable domain of the HL161B antibody with lysine (K). An HL161BKN antibody (RVT-1401) was also prepared by substituting the lysines (K) at positions 238 and 239 of the heavy chain (i.e., within the IgG1 heavy chain constant region) of the HL161BK antibody with alanines (A). Nucleotide sequences, amino acid sequences and CDR sequences of selected human FcRn antibodies are shown in Tables 1-5.

TABLE 1
Polynucleotide sequences of heavy chain and light chain variable
domains of selected human FcRn antibodies

| | Heavy chain variable domain | | Light chain variable domain | |
|---|---|---|---|---|
| | sequence | | sequence | |
| Antibody | SEQ | | SEQ ID | |
| name | ID NO. | Polynucleotide sequence | NO. | Polynucleotide sequence |
| HL161A | 1 | GAAGTGCAGC TGCTGGAATC | 11 | TCTTACGTGC TGACCCAGCC |
| | | CGGCGGAGGC CTGGTGCAGC | | CCCCTCCGTG TCTGTGGCTC |
| | | CTGGCGGCTC TCTGAGACTG | | CTGGCCAGAC CGCCAGAATC |
| | | TCCTGCGCCG CCTCCGAGTT | | ACCTGTGGCG GCAACAACAT |
| | | CACCTTCGGC AGCTGCGTGA | | CGGCTCCACC TCCGTGCACT |
| | | TGACCTGGGT CCGACAGGCT | | GGTATCAGCA GAAGCCCGGC |
| | | CCCGGCAAGG GCCTGGAATG | | CAGGCCCCCG TGCTGGTGGT |
| | | GGTGTCCGTG ATCTCCGGCT | | GCACGACGAC TCCGACCGGC |
| | | CCGGCGGCTC CACCTACTAC | | CTTCTGGCAT CCCTGAGCGG |
| | | GCCGACTCTG TGAAGGGCCG | | TTCTCCGGCT CCAACTCCGG |
| | | GTTCACCATC TCCCGGGACA | | CAACACCGCC ACCCTGACCA |
| | | ACTCCAAGAA CACCCTGTAC | | TCTCCAGAGT GGAAGCCGGC |
| | | CTGCAGATGA ACTCCCTGCG | | GACGAGGCCG ACTACTACTG |
| | | GGCCGAGGAC ACCGCCGTGT | | CCAAGTGCGA GACTCCTCCT |
| | | ACTACTGCGC CAAGACCCCC | | CCGACCACGT GATCTTCGGC |
| | | TGGTGGCTGC GGTCCCCCTT | | GGAGGCACCA AGCTGACCGT |
| | | CTTCGATTAC TGGGGCCAGG | | GCTGGGCCAG CCTAAGGCCG |
| | | GCACCCTGGT GACAGTGTCC | | CTCCCTCCGT GACCCTG |
| | | TCC | | |
| HL161B | 3 | CAACTGTTGC TCCAGGAATC | 13 | TCTTACGTGC TGACCCAGTC |
| | | CGGTCCTGGT CTTGTAAAGC | | CCCCTCCGTG TCCGTGGCTC |
| | | CATCTGAGAC TCTCTCCCTT | | CTGGCCAGAC CGCCAGAATC |
| | | ACCTGTACCG TTAGCGGAGG | | ACCTGTGGCG GCAACAACAT |
| | | AAGTCTTTCC TCAAGCTTCT | | CGGCTCCAAG TCCGTGCACT |
| | | CCTACTGGGT GTGGATCAGA | | GGTATCAGCA GAAGCCCGGC |
| | | CAGCCTCCCG GAAAAGGGTT | | CAGGCCCCCG TGCTGGTGGT |
| | | GGAGTGGATT GGCACAATAT | | GTACGACGAC TCCGACCGGC |
| | | ACTACTCCGG CAACACTTAC | | CCTCTGGCAT CCCTGAGCGG |
| | | TATAACCCCA GCCTGAAGAG | | TTCTCCGCCT CCAACTCCGG |
| | | CAGGCTGACT ATCTCTGTCG | | CAACACCGCC ACCCTGACCA |
| | | ACACCAGTAA AAATCACTTT | | TCTCCAGAGT GGAAGCCGGC |
| | | TCTCTGAATC TGTCTTCAGT | | GACGAGGCCG ACTACTACTG |
| | | GACCGCAGCC GACACCGCCG | | CCAAGTGTGG GACTCCTCCT |
| | | TGTATTATTG CGCTCGGCGC | | CCGACCACGT GGTGTTCGGC |
| | | GCCGGGATTC TGACAGGCTA | | GGAGGCACCA AGCTGACCGT |
| | | TCTGGATTCA TGGGGCCAGG | | GCTGGGCCAG CCTAAGGCCG |
| | | GGACATTGGT TACAGTGTCT | | CTCCCTCCGT GACCCTG |
| | | AGT | | |
| HL161BK | 5 | CAGCTGCTGC TGCAAGAATC | 15 | TCTTACGTGC TGACCCAGTC |
| (HL161BKN) | | CGGCCCTGGC CTGGTGAAAC | | CCCCTCCGTG TCCGTGGCTC |
| | | CCTCCGAGAC ACTGTCCCTG | | CTGGCCAGAC CGCCAGAATC |
| | | ACCTGCACCG TGTCCGGCGG | | ACCTGTGGCG GCAACAACAT |
| | | CTCCCTGTCC TCCAGCTTCT | | CGGCTCCAAG TCCGTGCACT |
| | | CCTACTGGGT CTGGATCCGG | | GGTATCAGCA GAAGCCCGGC |
| | | CAGCCCCCTG GCAAGGGCCT | | CAGGCCCCCG TGCTGGTGGT |
| | | GGAATGGATC GGCACCATCT | | GTACGACGAC TCCGACCGGC |
| | | ACTACTCCGG CAACACCTAC | | CCTCTGGCAT CCCTGAGCGG |
| | | TACAACCCCA GCCTGAAGTC | | TTCTCCGCCT CCAACTCCGG |
| | | CCGGCTGACC ATCTCCGTGG | | CAACACCGCC ACCCTGACCA |
| | | ACACCTCCAA GAACCACTTC | | TCTCCAGAGT GGAAGCCGGC |
| | | AGCCTGAAGC TGTCCTCCGT | | GACGAGGCCG ACTACTACTG |
| | | GACCGCCGCT GACACCGCCG | | CCAAGTGTGG GACTCCTCCT |
| | | TGTACTACTG TGCCAGAAGG | | CCGACCACGT GGTGTTCGGC |
| | | GCCGGCATCC TGACCGGCTA | | GGAGGCACCA AGCTGACCGT |
| | | CCTGGACTCT TGGGGCCAGG | | GCTGGGCCAG CCTAAGGCCG |
| | | GCACCCTGGT GACAGTGTCC | | CTCCCTCCGT GACCCTG |
| | | TCC | | |
| HL161C | 7 | CAGGTGCAGC TCGTGCAGTC | 17 | GACATCCAGA TGACCCAGTC |
| | | CGGCGCAGAG GTCAAAAAGC | | ACCATCATCC CTTTCCGCAT |
| | | CTGGTGCATC TGTGAAAGTG | | CTGTCGGAGA TAGAGTGACT |
| | | AGTTGCAAGG CTAGCGGCTA | | ATCACCTGCA GGGCTTCTCA |
| | | CACCTTTACC GGATGTTATA | | AGGTATTTCC AACTACCTCG |
| | | TGCATTGGGT ACGCCAAGCC | | CCTGGTTCCA GCAAAAGCCA |
| | | CCCGGACAAG GCTTGGAATG | | GGTAAAGGCC CAAAGAGGTT |
| | | GATGGGGCGT ATCAACCCAA | | GATCTACGCC GCTTCTAGTC |
| | | ACTCTGGCGG GACTAATTAC | | TGCAGAGTGG AGTTCCTAGT |
| | | GCCCAGAAGT TTCAGGGAAG | | AAGTTCTCCG GCTCTGGCAG |
| | | GGTGACTATG ACAAGGGACA | | TGGCACAGAT TTTACCTTGA |
| | | CATCCATATC CACCGCTTAT | | CCATTTCCAG CCTGCAGTCT |
| | | ATGGACCTGT CTCGACTGCG | | GAGGATTTCG CTACCTACTA |
| | | GTCTGATGAT ACAGCCGTTT | | TTGTCAGCAG TATGACAGCT |
| | | ATTACTGCGC CAGAGACTAC | | ATCCCCCCAC ATTTGGGGGG |
| | | AGCGGATGGA GCTTCGATTA | | GGCACTAAGG TGGAGATAAA |
| | | TTGGGGGCAG GGTACTTTGG | | ACGGACAGTG GCTGCCCCTT |
| | | TCACAGTTTC AAGT | | CTGTCTTTAT T |
| HL161D | 9 | CAGCTGCAGT TGCAGGAGTC | 19 | AGGTATGAGC TGACCCAGCC |
| | | AGGCCCCGGT TTGGTTAAGC | | TCTGAGCGTA TCTGTCGCTC |
| | | CTTCTGAAAC CCTTTCTCTC | | TCGGCCAGAC AGCCAGAATT |
| | | ACATGCACAG TATCCGGTGG | | ACCTGTGGCG GCAATAACAT |
| | | CTCCATCTCC AGTTCAAGTT | | AGGATCCAAA AATGTTCACT |
| | | ACTACTGGGG ATGGATCCGG | | GGTATCAGGA AAAACCTGGC |
| | | CAACCCCCAG GAAAAGGGCT | | CAAGCTCCCG TGCTCGTGAT |
| | | GGAGTGGATT GGCAATATAT | | CTACCGGGAC TCTAACCGAC |
| | | ATTACTCTGG GTCCACCTAT | | CCAGTGGAAT CCCCGAACGC |
| | | TACAACCCTT CCCTGATGAG | | TTTAGCGGTT CCAACTCTGG |
| | | TAGAGTGACC ATCAGCGTGG | | AAATACAGCT ACTCTGACTA |
| | | ACACAAGCAA AAACCAATTC | | TCTCCAGGGC TCAGGCCGGG |
| | | AGCCTGAAGC TTTCTAGCGT | | GATGAGGCCG ATTACTACTG |
| | | GACCGCTGCC GACACAGGTG | | CCAGGTGTGG GACTCAAGCA |
| | | TCTATTACTG TGCCCGCCAG | | CAGTGGTCTT CGGCGGAGGT |
| | | CTTAGTTATA ACTGGAATGA | | ACCAAGTTGA CTGTTCTTGG |
| | | TAGGCTGTTT GATTACTGGG | | GCAGCCAAAG GCCGCACCTT |
| | | GCCAGGGGAC TCTCGTTACA | | CAGTGACCCT G |
| | | GTCAGCAGC | | |

TABLE 2
Amino acid sequences of heavy chain and light chain variable domains
of selected human FcRn antibodies

| | Heavy chain variable domain | | Light chain variable domain | |
|---|---|---|---|---|
| | sequence | | sequence | |
| Antibody | SEQ | | SEQ ID | |
| name | ID NO. | Amino acid sequence | NO. | Amino acid sequence |
| HL161A | 2 | EVQLLESGGG LVQPGGSLRL | 12 | SYVLTQPPSV SVAPGQTARI |
| | | SCAASEFTFG SCVMTWVRQA | | TCGGNNIGST SVHWYQQKPG |
| | | PGKGLEWVSV ISGSGGSTYY | | QAPVLVVHDD SDRPSGIPER |
| | | ADSVKGRFTI SRDNSKNTLY | | FSGSNSGNTA TLTISRVEAG |
| | | LQMNSLRAED TAVYYCAKTP | | DEADYYCQVR DSSSDHVIFG |
| | | WWLRSPFFDY WGQGTLVTVSS | | GGTKLTVLGQ PKAAPSVTL |
| HL161B | 4 | QLLLQESGPG LVKPSETLSL | 14 | SYVLTQSPSV SVAPGQTARI |
| | | TCTVSGGSLS SSFSYWVWIR | | TCGGNNIGSK SVHWYQQKPG |
| | | QPPGKGLEWI GTIYYSGNTY | | QAPVLVVYDD SDRPSGIPER |
| | | YNPSLKSRLT ISVDTSKNHF | | FSASNSGNTA TLTISRVEAG |
| | | SLNLSSVTAA DTAVYYCARR | | DEADYYCQVW DSSSDHWFG |
| | | AGILTGYLDS WGQGTLVTVSS | | GGTKLTVLGQ PKAAPSVTL |
| HL161BK | 6 | QLLLQESGPG LVKPSETLSL | 16 | SYVLTQSPSV SVAPGQTARI |
| (HL161BKN) | | TCTVSGGSLS SSFSYWVWIR | | TCGGNNIGSK SVHWYQQKPG |
| | | QPPGKGLEWI GTIYYSGNTY | | QAPVLVVYDD SDRPSGIPER |
| | | YNPSLKSRLT ISVDTSKNHF | | FSASNSGNTA TLTISRVEAG |
| | | SLKLSSVTAA DTAVYYCARR | | DEADYYCQVW DSSSDHWFG |
| | | AGILTGYLDS WGQGTLVTVSS | | GGTKLTVLGQ PKAAPSVTL |
| HL161C | 8 | QVQLVQSGAE VKKPGASVKV | 18 | DIQMTQSPSS LSASVGDRVT |
| | | SCKASGYTFT GCYMHWVRQA | | ITCRASQGIS NYLAWFQQKP |
| | | PGQGLEWMGR INPNSGGTNY | | GKAPKSLIYA ASSLQSGVPS |
| | | AQKFQGRVTM TRDTSISTAY | | KFSGSGSGTD FTLTISSLQS |
| | | MDLSRLRSDD TAVYYCARDY | | EDFATYYCQQ YDSYPPTFGG |
| | | SGWSFDYWGQ GTLVTVSS | | GTKVEIKRTV AAPSVFI |
| HL161D | 10 | QLQLQESGPG LVKPSETLSL | 20 | SYELTQPLSV SVALGQTARI |
| | | TCTVSGGSIS SSSYYWGWIR | | TCGGNNIGSK NVHWYQQKPG |
| | | QPPGKGLEWI GNIYYSGSTY | | QAPVLVIYRD SNRPSGIPER |
| | | YNPSLMSRVT ISVDTSKNQF | | FSGSNSGNTA TLTISRAQAG |
| | | SLKLSSVTAA DTAVYYCARQ | | DEADYYCQVW DSSTWFGGG |
| | | LSYNWNDRLF DYWGQGTLVT | | TKLTVLGQPK AAPSVTL |
| | | VSS | | |

TABLE 3
Polynucleotide sequences of full-length heavy chain and light chain of
selected human FcRn antibodies

| | Heavy chain sequence | | Light chain sequence | |
|---|---|---|---|---|
| Antibody | SEQ ID | | SEQ ID | |
| name | NO. | Polynucleotide sequence | NO. | Polynucleotide sequence |
| HL161BKN | 45 | CAG CTG CTG CTG CAA | 47 | TCT TAC GTG CTG ACC |
| | | GAA TCC GGC CCT GGC | | CAG TCC CCC TCC GTG |
| | | CTG GTG AAA CCC TCC | | TCC GTG GCT CCT GGC |
| | | GAG ACA CTG TCC CTG | | CAG ACC GCC AGA ATC |
| | | ACC TGC ACC GTG TCC | | ACC TGT GGC GGC AAC |
| | | GGC GGC TCC CTG TCC | | AAC ATC GGC TCC AAG |
| | | TCC AGC TTC TCC TAC | | TCC GTG CAC TGG TAT |
| | | TGG GTC TGG ATC CGG | | CAG CAG AAG CCC GGC |
| | | CAG CCC CCT GGC AAG | | CAG GCC CCC GTG CTG |
| | | GGC CTG GAA TGG ATC | | GTG GTG TAC GAC GAC |
| | | GGC ACC ATC TAC TAC | | TCC GAC CGG CCC TCT |
| | | TCC GGC AAC ACC TAC | | GGC ATC CCT GAG CGG |
| | | TAC AAC CCC AGC CTG | | TTC TCC GCC TCC AAC |
| | | AAG TCC CGG CTG ACC | | TCC GGC AAC ACC GCC |
| | | ATC TCC GTG GAC ACC | | ACC CTG ACC ATC TCC |
| | | TCC AAG AAC CAC TTC | | AGA GTG GAA GCC GGC |
| | | AGC CTG AAG CTG TCC | | GAC GAG GCC GAC TAC |
| | | TCC GTG ACC GCC GCT | | TAC TGC CAA GTG TGG |
| | | GAC ACC GCC GTG TAC | | GAC TCC TCC TCC GAC |
| | | TAC TGT GCC AGA AGG | | CAC GTG GTG TTC GGC |
| | | GCC GGC ATC CTG ACC | | GGA GGC ACC AAG CTG |
| | | GGC TAC CTG GAC TCT | | ACC GTG CTG GGC CAG |
| | | TGG GGC CAG GGC ACC | | CCT AAG GCC GCT CCC |
| | | CTG GTG ACA GTG TCC | | TCC GTG ACC CTG TTC |
| | | TCC GCC TCC ACC AAG | | CCC CCA TCC TCC GAG |
| | | GGC CCC TCC GTG TTC | | GAA CTG CAG GCC AAC |
| | | CCT CTG GCC CCC TCC | | AAG GCC ACC CTG GTC |
| | | AGC AAG TCC ACC TCT | | TGC CTG ATC TCC GAC |
| | | GGC GGC ACC GCT GCC | | TTC TAC CCT GGC GCC |
| | | CTG GGC TGT CTG GTG | | GTG ACC GTG GCC TGG |
| | | AAA GAC TAC TTC CCC | | AAG GCC GAC AGC TCT |
| | | GAG CCC GTG ACC GTG | | CCT GTG AAG GCC GGC |
| | | TCC TGG AAC TCT GGC | | GTG GAA ACC ACC ACC |
| | | GCC CTG ACC TCC GGC | | CCC TCC AAG CAG TCC |
| | | GTG CAC ACC TTC CCT | | AAC AAC AAA TAC GCC |
| | | GCC GTG CTG CAG TCC | | GCC TCC TCC TAC CTG |
| | | TCC GGC CTG TAC TCC | | TCC CTG ACC CCC GAG |
| | | CTG TCC AGC GTG GTG | | CAG TGG AAG TCC CAC |
| | | ACC GTG CCC TCC AGC | | CGG TCC TAC AGC TGC |
| | | TCT CTG GGC ACC CAG | | CAA GTG ACA CAC GAG |
| | | ACC TAC ATC TGC AAC | | GGC TCC ACC GTG GAA |
| | | GTG AAC CAC AAG CCC | | AAG ACC GTG GCC CCT |
| | | TCC AAC ACC AAG GTG | | ACC GAG TGC TCC |
| | | GAC AAG CGG GTG GAA | | |
| | | CCC AAG TCC TGC GAC | | |
| | | AAG ACC CAC ACC TGT | | |
| | | CCC CCC TGT CCT GCC | | |
| | | CCT GAA GCT GCT GGC | | |
| | | GGC CCT AGC GTG TTC | | |
| | | CTG TTC CCC CCA AAG | | |
| | | CCC AAG GAC ACC CTG | | |
| | | ATG ATC TCC CGG ACC | | |
| | | CCC GAA GTG ACC TGC | | |
| | | GTG GTG GTG GAC GTG | | |
| | | TCC CAC GAG GAC CCT | | |
| | | GAA GTG AAG TTC AAT | | |
| | | TGG TAC GTG GAC GGC | | |
| | | GTG GAA GTG CAC AAC | | |
| | | GCC AAG ACC AAG CCC | | |
| | | AGA GAG GAA CAG TAC | | |
| | | AAC TCC ACC TAC CGG | | |
| | | GTG GTG TCC GTG CTG | | |
| | | ACC GTG CTG CAC CAG | | |
| | | GAC TGG CTG AAC GGC | | |
| | | AAA GAG TAC AAG TGC | | |
| | | AAG GTC TCC AAC AAG | | |
| | | GCC CTG CCT GCC CCC | | |
| | | ATC GAA AAG ACC ATC | | |
| | | TCC AAG GCC AAG GGC | | |
| | | CAG CCC CGC GAG CCC | | |
| | | CAG GTG TAC ACA CTG | | |
| | | CCC CCT AGC CGG GAA | | |
| | | GAG ATG ACC AAG AAC | | |
| | | CAG GTG TCC CTG ACA | | |
| | | TGC CTG GTG AAG GGC | | |
| | | TTC TAC CCC TCC GAC | | |
| | | ATT GCC GTG GAA TGG | | |
| | | GAG TCC AAC GGC CAG | | |
| | | CCC GAG AAC AAC TAC | | |
| | | AAG ACC ACC CCC CCT | | |
| | | GTG CTG GAC TCC GAC | | |
| | | GGC TCA TTC TTC CTG | | |
| | | TAC TCC AAG CTG ACC | | |
| | | GTG GAC AAG TCC CGG | | |
| | | TGG CAG CAG GGC AAC | | |
| | | GTG TTC TCC TGC TCC | | |
| | | GTG ATG CAC GAG GCC | | |
| | | CTG CAC AAC CAC TAC | | |
| | | ACC CAG AAG TCC CTG | | |
| | | TCC CTG AGC CCC GGC | | |

TABLE 4
Amino acid sequences of full-length heavy chain and light chain of
selected human FcRn antibodies

| | Heavy chain sequence | | Light chain sequence | |
|---|---|---|---|---|
| Antibody | SEQ ID | | SEQ ID | |
| name | NO. | Amino acid sequence | NO. | Amino acid sequence |
| HL161BKN | 46 | QLLLQESGPG LVKPSETLSL | 48 | SYVLTQSPSV SVAPGQTARI |
| | | TCTVSGGSLS SSFSYWVWIR | | TCGGNNIGSK SVHWYQQKPG |
| | | QPPGKGLEWI GTIYYSGNTY | | QAPVLVVYDD SDRPSGIPER |
| | | YNPSLKSRLT ISVDTSKNHF | | FSASNSGNTA TLTISRVEAG |
| | | SLKLSSVTAA DTAVYYCARR | | DEADYYCQVW DSSSDHWFG |
| | | AGILTGYLDS WGQGTLVTVS | | GGTKLTVLGQ PKAAPSVTLF |
| | | SASTKGPSVF PLAPSSKSTS | | PPSSEELQAN KATLVCLISD |
| | | GGTAALGCLV KDYFPEPVTV | | FYPGAVTVAW KADSSPVKAG |
| | | SWNSGALTSG VHTFPAVLQS | | VETTTPSKQS NNKYAASSYL |
| | | SGLYSLSSW TVPSSSLGTQ | | SLTPEQWKSH RSYSCQVTHE |
| | | TYICNVNHKP SNTKVDKRVE | | GSTVEKTVAP TECS |
| | | PKSCDKTHTC PPCPAPEAAG | | |
| | | GPSVFLFPPK PKDTLMISRT | | |
| | | PEVTCVVVDV SHEDPEVKFN | | |
| | | WYVDGVEVHN AKTKPREEQY | | |
| | | NSTYRWSVL TVLHQDWLNG | | |
| | | KEYKCKVSNK ALPAPIEKTI | | |
| | | SKAKGQPREP QVYTLPPSRE | | |
| | | EMTKNQVSLT CLVKGFYPSD | | |
| | | IAVEWESNGQ PENNYKTTPP | | |
| | | VLDSDGSFFL YSKLTVDKSR | | |
| | | WQQGNVFSCS VMHEALHNHY | | |
| | | TQKSLSLSPG | | |

TABLE 5
CDR sequences of heavy chain and light chain variable domains of
selected human FcRn antibodies

| | Heavy chain variable domain CDR | | | Light chain variable domain CDR | | |
|---|---|---|---|---|---|---|
| Antibody | CDR1 | CDR2 | CDR3 | CDR1 | CDR2 | CDR3 |
| SEQ ID NO. | 21 | 22 | 23 | 24 | 25 | 26 |
| HL161A | SCVMT | VISGSGGST | TPWWLRSPF | GGNNIGST | DDSDRPS | VRDSSSDHV |
| | | YYADSVKG | FDY | SVH | | I |
| SEQ ID NO. | 27 | 28 | 29 | 30 | 31 | 32 |
| HL161B | FSYWV | TIYYSGNTY | RAGILTGYL | GGNNIGSK | DDSDRPS | QVWDSSSDH |
| (HL161BK) | | YNPSLKS | DS | SVH | | VV |
| (HL161BKN) | | | | | | |
| SEQ ID NO. | 33 | 34 | 35 | 36 | 37 | 38 |
| HL161C | GCYMH | RINPNSGGT | DYSGWSFDY | RASQGISN | AASSLQS | QQYDSYPPT |
| | | NYAQKFQG | | YLA | | F |
| SEQ ID NO. | 39 | 40 | 41 | 42 | 43 | 44 |
| HL161D | SYYWG | NIYYSGSTY | QLSYNWNDR | GGNNIGSK | RDSNRPS | QVWDSSTVV |
| | | YNPSLMS | LFDY | NVH | | |

### Example 4: Measurement of Antigen Binding Affinity of HL161A, HL161B, HL161C, and HL161D Antibodies by Surface Plasmon Resonance (SPR)

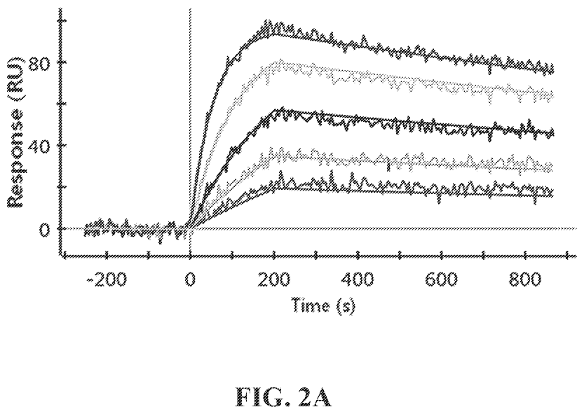

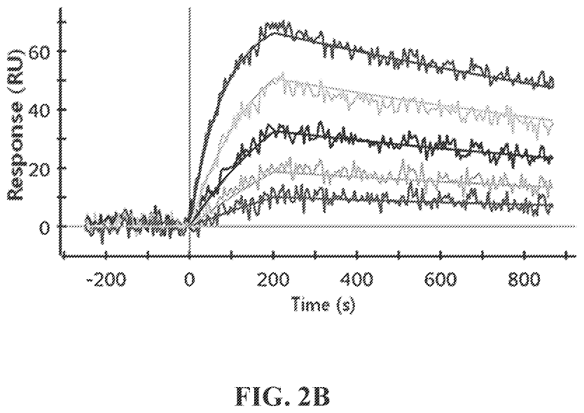

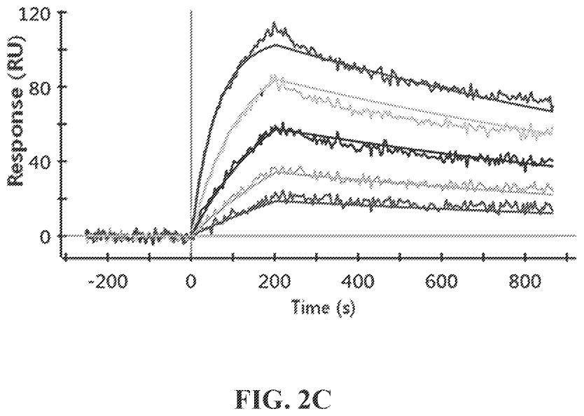

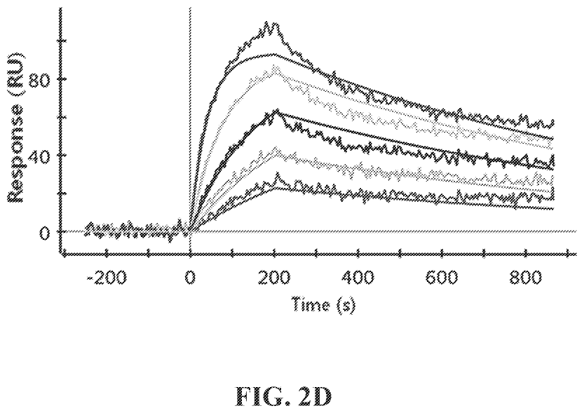

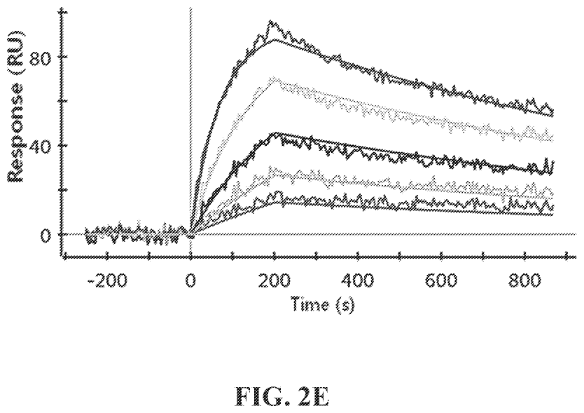

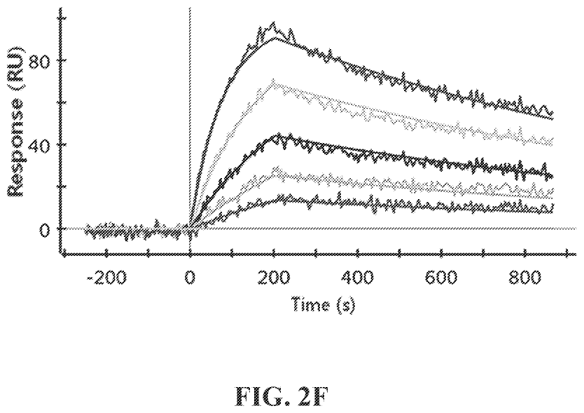

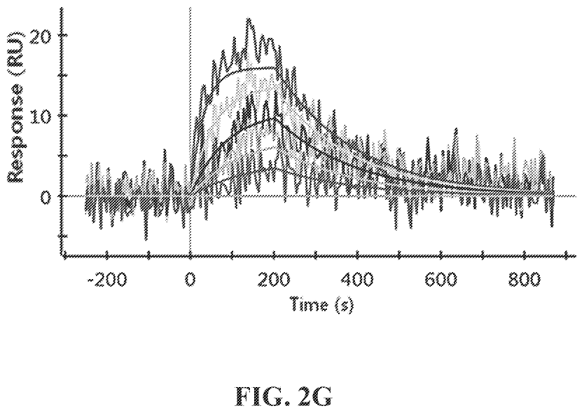

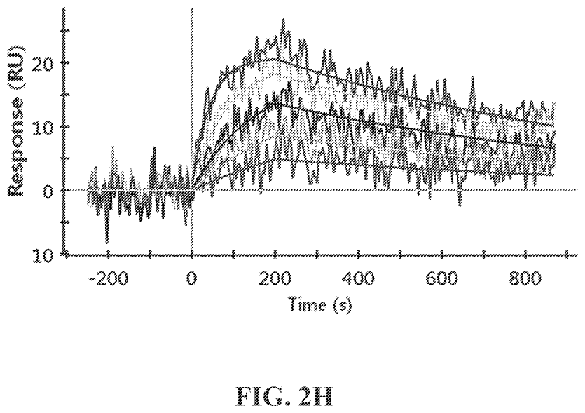

The binding affinities of HL161A, HL161B, HL161C and HL161D antibodies were measured by SPR by immobilizing water-soluble hFcRn as a ligand onto a Proteon GLC chip (Bio-Rad) and measuring the affinity. Kinetic analysis was performed using a Proteon XPR36 system. Water-soluble human FcRn (shFcRn) was immobilized on a GLC chip, and an antibody sample was allowed to react at a concentration of 5, and sensogram results were obtained. In kinetic analysis, a 1:1 Langmuir binding model was used, the analysis was repeated six times at each of pH 6.0 and pH 7.4, and the mean K_{D} value was calculated. Following the immobilization step, the chip was activated under the conditions of EDAC/NHS 0.5×, 30 μL/min and 300 seconds. For immobilization, shFcRn was diluted in acetate buffer (pH 5.5) to concentrations of 2 μg/mL and 250 μL, and the dilution was allowed to flow on the chip at a rate of 30 μL/min. When an immobilization level of 200-300 RU was reached, the reaction was stopped. Then, deactivation was performed using ethanolamine at a rate of 30 μL/min for 300 seconds. Each of the HL161 antibodies was serially 2-fold diluted from a concentration of 10 nM to 5 nM, 2.5 nM, 1.25 nM, 0.625 nM, 0.312 nM, etc., thereby preparing samples. Sample dilution was performed using 1×PBST (pH 7.4) or 1×PBST (pH 6.0) at each pH. For sample analysis, association was performed at 50 μL/min for 200 seconds, and the dissociation step was performed at 50 L/min for 600 seconds, after which regeneration was performed using glycine buffer (pH 2.5) at 100 μL/min for 18 seconds. The kinetic analysis of each sample was repeated six times, and then the mean antigen binding affinity (K_{D}) was measured. The kinetic parameters of the antibodies, which resulted from the SPR analysis, are shown in Table 6 (FIG. 2A to FIG. 2H).

TABLE 6
Results of kinetic analysis of antibody by human FcRn-immobilized SPR

| | pH 6.0 | | | pH 7.4 | | |
|---|---|---|---|---|---|---|
| Antibody | kₒₙ (M⁻¹s⁻¹) | k_{off}(s⁻¹) | K_{D} (M) | kₒₙ (M⁻¹s⁻¹) | k_{off}(s⁻¹) | K_{D} (M) |
| HL161A | 1.81 × 10⁶ | 3.26 × 10⁻⁴ | 1.80 × 10⁻¹⁰ | 1.32 × 10⁶ | 3.27 × 10⁻⁴ | 2.47 × 10⁻¹⁰ |
| HL161B | 9.12 × 10⁵ | 7.35 × 10⁻⁴ | 8.07 × 10⁻¹⁰ | 7.10 × 10⁵ | 1.25 × 10⁻³ | 1.76 × 10⁻⁹ |
| HL161C | 1.74 × 10⁶ | 3.32 × 10⁻⁴ | 1.91 × 10⁻¹⁰ | 1.36 × 10⁶ | 3.16 × 10⁻⁴ | 2.32 × 10⁻¹⁰ |
| HL161D | 9.70 × 10⁵ | 1.38 × 10⁻³ | 1.43 × 10⁻⁹ | 6.99 × 10⁵ | 1.24 × 10⁻³ | 1.78 × 10⁻⁹ |
| hIgG₁ | 3.2 × 10⁵ | 4.6 × 10⁻⁴ | 1.4 × 10⁻⁹ | No binding | No binding | No binding |

### Example 5: Analysis of Binding of HL161A and HL161B Antibodies to Human FcRn by FACS

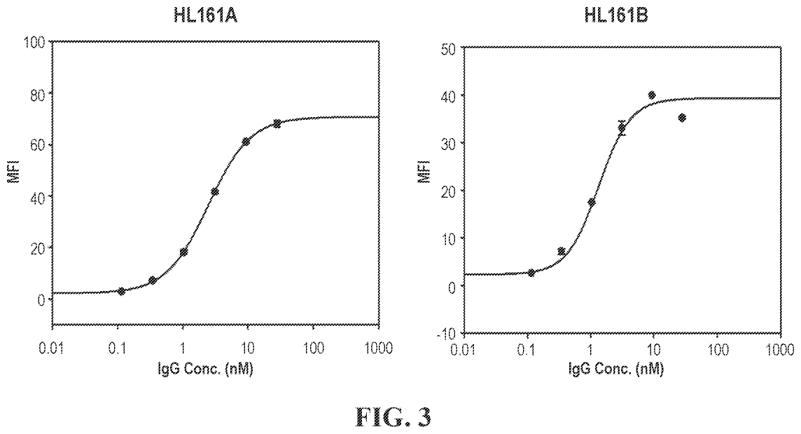

Using human FcRn-expressing stable HEK293 cells, binding to FcRn at each pH was analyzed using a FACS system. The FcRn binding test using FACS was performed in reaction buffer at pH 6.0 and pH 7.4. Specifically, 100,000 human FcRn-expressing stable HEK293 cells were washed with PBS buffer and centrifuged in a table microcentrifuge at 4500 rpm for 5 minutes to obtain cell pellets. The antibody was added to 100 μL of pH 6.0 or pH 7.4 PBS/10 mM EDTA. The remaining cell pellets were suspended in reaction buffer, and cell counting was performed. 10 μL of the cell suspension was added to a slide, and the number of the cells in the cell suspension was counted in a TC10 system, after which the cell suspension was diluted with reaction buffer to a cell concentration of 2×10⁶ cells/mL. Each antibody sample was diluted to 500 nM. For analysis at pH 6.0, the dilution was diluted to 20 nM in a 96-well v-bottom plate, and 50 μL of the dilution was added to each well. For analysis at pH 7.4, 500 nM antibody sample was diluted by 3-fold serial dilution, and analyzed at a concentration ranging from 250 nM to 0.11 nM. 50 μL of the cells diluted to 2×10⁶ cells/mL were added to each well and suspended. The plate was mounted in a rotator at 4° C. and rotated at an angle of 15° and 10 rpm for 90 minutes. After completion of the reaction, the plate was taken out of the rotator and centrifuged at 2000 rpm for 10 minutes, and the supernatant was removed. A488 anti-hIgG goat antibody was diluted at 1:200 in reaction buffer, and 100 μL of the antibody dilution was added to each well and suspended. Next, the plate was mounted again in a rotator at 4° C. and rotated at an angle of 15° and 10 rpm for 90 minutes. After completion of the reaction, the plate was taken out of the rotator and centrifuged at 2000 rpm for 10 minutes, and the supernatant was removed. After the washing procedure was performed once more, 100 μL of reaction buffer was added to each well to dissolve the cell pellets, and the plate was transferred into a blue test tube. Next, 200 μL of reaction buffer was added to each well, and then measurement was performed in FACS. The FACS measurement was performed under the following conditions: FS 108 volts, SS 426 volts, FL1 324 volts, FL2 300 volts. These cells were analyzed by FACS using BD FACSDivam v6.1.3 software (BD Bioscience). The results were expressed as Mean Fluorescence Intensity (MFI) (FIG. 3). The HL161A and HL161B antibodies showed MFI values of 10.59 and 8.34, respectively, at a concentration of 10 nM and pH 6.0. At pH 7.4 and a concentration of 0.11-250 nM, the antibodies showed EC50 (Effective Concentration 50%) values of 2.46 nM and 1.20 nM, respectively, as analyzed by 4 parameter logistic regression using the MFI values.

### Example 6: Analysis of Blocking Effects of HL161A and HL161B Antibodies by FACS

HEK293 cells that express hFcRn on the cell surface were treated with the HL161A and HL161B antibodies (previously analyzed for their binding affinity for cell surface human FcRn), and the blocking effects of the antibodies were examined based on a reduction in the binding of Alexa-Fluo-488-labeled hIgG1. The analysis procedure was performed in the following manner:

2 mL of 1×TE was added to each type of naïve HEK293 cells and human FcRn-overexpressing stable HEK293 cells, which were incubated in a 5% CO₂ incubator at 37° C. for 1 minute. The cells were recovered from the flasks, and 8 mL of reaction buffer (pH 6.0) was added thereto, after which the cells were transferred into a 50 mL conical tube. The cell suspension was centrifuged at 2000 rpm for 5 minutes to remove the supernatant, and 1 mL of reaction buffer (pH 6.0) was added to each cell pellet. Then, the cell suspension was transferred into a fresh 1.5 mL Eppendorf tube. Next, the cell suspension was centrifuged at 4000 rpm for 5 minutes, and the supernatant was removed. Then, reaction buffer (pH 6.0) was added to the remaining cell pellet, and the cell number of the cell suspension was counted. Finally, the cell suspension was diluted with reaction buffer to a cell concentration of 2.5×10⁶ cells/mL.

Each antibody sample was diluted to 400 nM, and then diluted by 4-fold serial dilution in a 96-well v-bottom plate. 50 μL of the sample diluted to a final concentration of 200 nM to 0.01 nM was added to each well. Then, 10 μL of Alex488-hIgG1 diluted with 1 μM reaction buffer (pH 6.0) was each well. Finally, 40 μL of cells diluted to a cell concentration of 2.5×10⁶ cells/mL were added to each well and suspended. The plate was mounted in a rotator at 4° C. and rotated at an angle of 15° and 10 rpm for 90 minutes. After completion of the reaction, the plate was taken out of the rotator, and centrifuged at 2000 rpm for 10 minutes to remove the supernatant. 100 μL of reaction buffer was added to each well to dissolve the cell pellets, and the plate was transferred into a blue test tube. Then, 200 μL of reaction buffer was added to each well, and measurement was performed in FACS. The FACS measurement was performed under the following conditions: FS 108 volts, SS 426 volts, FL1 324 volts, FL2 300 volts. These cells were analyzed by FACS using BD FACSDiva™ v6.1.3 software (BD Bioscience). The results were expressed as mean fluorescence intensity (MFI). The MFI of the test group was processed after subtracting the measured MFI value of the cells alone (background signal). The percentage of the MFI of the competitor-containing tube relative to 100% of a control tube (Alexa Fluor 488 alone, and no competitor) was calculated.

$$Blocking ( \% ) = \{ \frac{\begin{matrix}MFI of hFcRn stable ( Competitor + A 488 ‐ hIgG 1 ) - \\ MFI of HEK 293 ( A 488 ‐ hIgG 1 )\end{matrix}}{\begin{matrix}MFI of hFcRn stable ( A 488 ‐ hIgG 1 ) \cdot \\ MFI of HEK 293 ( A 488 ‐ hIgG 1 )\end{matrix}} \} \times 100$$

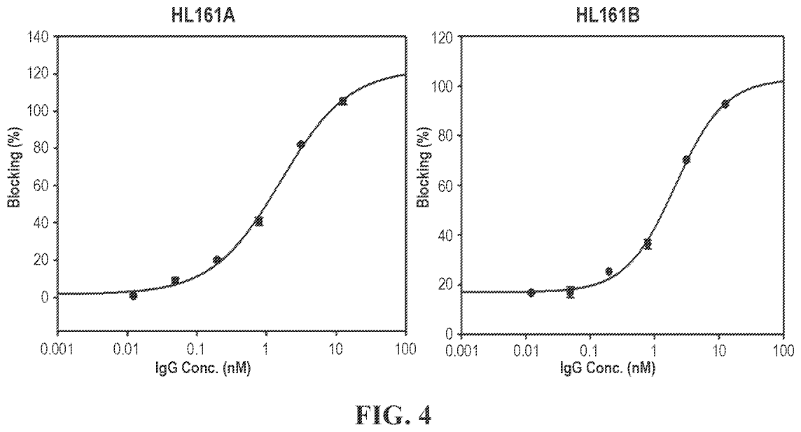

When the MFI was lower than the MFI of the human IgG1 competitor-containing tube, the competitor antibody was determined to have high competition rate. Based on the measured blocking effects (%) of the HL161A and HL161B antibodies under the conditions of pH 6.0 and concentration of 0.01-200 nM, 4-parameter logistic regression was performed. As a result, it was shown that the HL161A and HL161B antibodies showed IC50 (Inhibitory Concentration 50%) values of 0.92 nM and 2.24 nM, respectively (FIG. 4).

### Example 7: Test for Effects of HL161A and HL161B in mFcRn−/−hFCRN Transgenic 32 (Tg32) Mice

Human IgG was injected into human FcRn-expressing Tg32 (hFcRn+/+, hβ2m+/+, mFcRn−/−, mβ2m−/−) mice (Jackson Laboratory), and then HL161A and HL161B together with human IgG were administered to the mice in order to examine whether the antibodies would influence the catabolism of human IgG.

HL161A and HL161B antibodies and human IgG (Greencross, IVglobulinS) were dispensed for 4-day administration at doses of 5, 10 and 20 mg/kg and stored, and PBS (phosphate buffered saline) buffer (pH 7.4) was used as a vehicle and a 20 mg/kg IgG1 control. Human FcRn Tg32 mice were adapted for about 7 days and given water and feed ad libitum. Temperature (23±2° C.), humidity (55±5%) and 12-hr-light/12-hr-dark cycles were automatically controlled. Each animal group consisted of 4 mice. To use human IgG as a tracer, biotin-conjugated hIgG was prepared using a kit (Pierce, Cat #. 21327). At 0 hour, 5 mg/kg of biotin-hIgG and 495 mg/kg of human IgG were administered intraperitoneally to saturate IgG in vivo. At 24, 48, 72 and 96 hours after administration of biotin-IgG, each drug was injected intraperitoneally at doses of 5, 10 and 20 mg/kg once a day. For blood collection, the mice were lightly anesthetized with Isoflurane (JW Pharmaceutical), and then blood was collected from the retro-orbital plexus using a heparinized Micro-hematocrit capillary tube (Fisher) at 24, 48, 72, 96, 120 and 168 hours after administration of biotin-IgG. At 24, 48, 72 and 96 hours, the drug was administered after blood collection. Immediately after 0.1 mL of whole blood was received in an Eppendorf tube, plasma was separated by centrifugation and stored in a deep freezer (Thermo) at −70° C. until analysis.

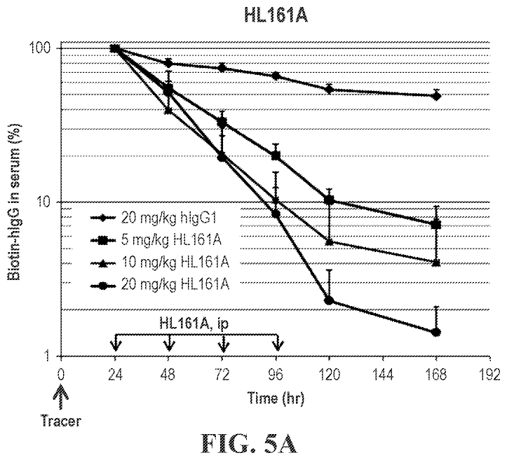

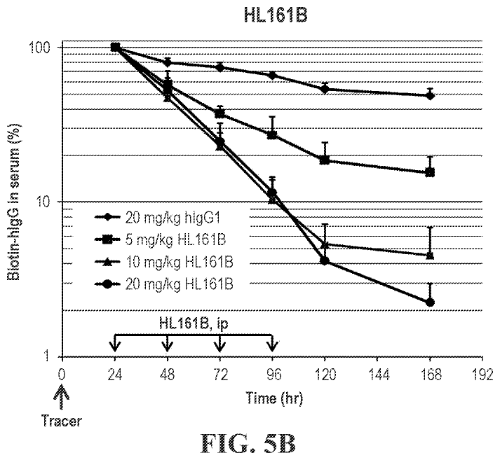

The level of biotin-hIgG1 in the collected blood was analyzed by ELISA in the following manner. 100 μL of Neutravidin (Pierce, 31000) was added to a 96-well plate (Costar, Cat. No: 2592) to a concentration of 1.0 μg/mL, and then coated at 4° C. for 16 hours. The plate was washed three times with buffer A (0.05% Tween-20, 10 mM PBS, pH 7.4), and then incubated in 1% BSA-containing PBS (pH 7.4) buffer at room temperature for 2 hours. Next, the plate was washed three times with buffer A, and then a Neutravidin plate was prepared with 0.5% BSA-containing PBS (pH 7.4) buffer so as to correspond to 1 g/mL. A blood sample was serially diluted 500-1000-fold in buffer B (100 mM MES, 150 mM NaCl, 0.5% BSA IgG-free, 0.05% Tween-20, pH 6.0), and 150 μL of the dilution was added to each well of the plate. The added sample was allowed to react at room temperature for 1 hour. Next, the plate was washed three times with buffer A, and then 200 μL of 1 nM HRP-conjugated anti-human IgG goat antibody was added to each well and incubated at 37° C. for 2 hours. Next, the plate was washed three times with ice cold buffer B, and then 100 μL of the substrate solution tetramethylbenzidine (RnD, Cat. No: DY999) was added to each well and allowed to react at room temperature for 15 minutes. 50 μL of 1.0 M sulfuric acid solution (Samchun, Cat. No: 52129) was added to each well to stop the reaction, after which the absorbance at 450 nm was measured. The concentration of biotin-IgG after 24 hours (approximately Tmax of biotin-IgG in mice; before the occurrence of catabolism of biotin-IgG) was set at 100%, and the percentages of the concentration at other time points relative to the concentration at 24 hours were analyzed. The half-lives of the vehicle and the 20 mg/kg IgG1 control were 103 hours and 118 hours, respectively. The IgG half-lives of the HL161A antibody were 30, 23, and 18 hours at varying doses. In addition, the HL161B antibody showed IgG half-lives of 41, 22, and 21 hours (FIG. 5A and FIG. 5B).

### Example 8: Test for Effects of HL161A and HL161B in Monkeys

Using cynomolgus monkeys having a homology of 96% to human FcRn, the monkey IgG, IgA, IgM and albumin levels by administration of the HL161A and HL161B antibodies were analyzed, and the pharmacokinetics (PK) profiles of the antibodies were analyzed.

1) Analysis of Change in Expression of Immunoglobulin G in Monkey Blood

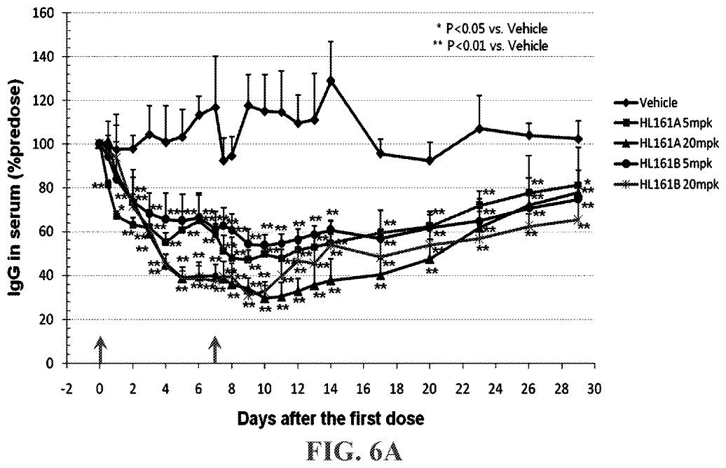

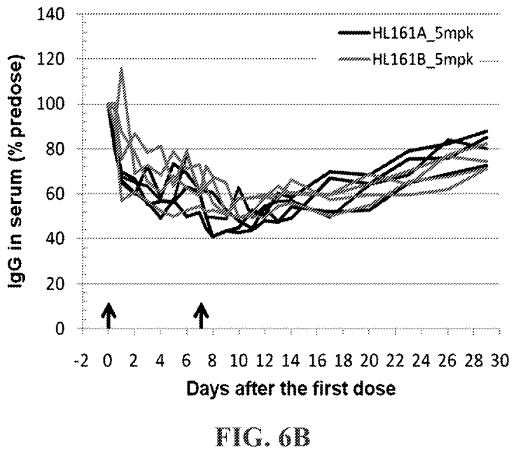

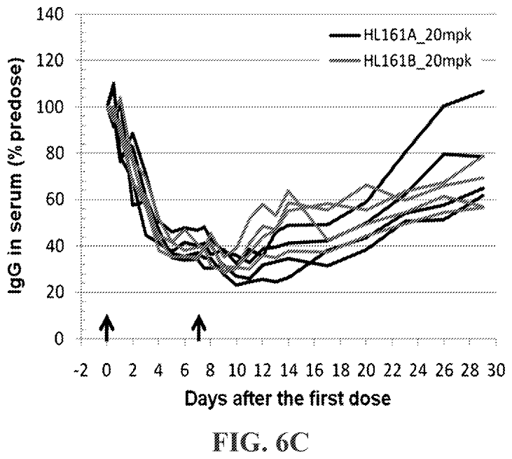

First, a change in monkey IgG was measured by ELISA analysis. 100 μL of anti-human IgG Fe antibody (BethylLab, A80-104A) was loaded into each well of a 96-well plate (Costar, Cat. No: 2592) to a concentration of 4.0 μg/mL, and then coated at 4° C. for 16 hours. The plate was washed three times with washing buffer (0.05% Tween-20, 10 mM PBS, pH 7.4), and then incubated with 1% BSA-containing PBS (pH 7.4) buffer at room temperature for 2 hours. The standard monkey IgG was used at a concentration of 3.9-500 ng/mL, and the blood sample was diluted 80,000-fold in 1% BSA-containing PBS (pH 7.4) buffer, and the dilution was loaded into the plate and incubated at room temperature for 2 hours. Next, the plate was washed three times with washing buffer, and then 100 μL of a 20,000-fold dilution of anti-hIgG antibody (Biorad, 201005) was loaded into the plate and allowed to react at room temperature for 1 hour. After each plate was washed, 100 μL of the substrate solution 3,3′,5,5′-tetramethylbenzidine (RnD, Cat. No: DY999) was loaded into the plate and allowed to react at room temperature for 7 minutes, after which 50 μL of 1.0 M sulfuric acid solution (Samchun, Cat. No: S2129) was added to each well to stop the reaction. For analysis, absorbance (OD) was measured using a 450 nm and 540 nm absorbance reader (MD, Model: VersaMax). Change (%) in monkey IgG level by administration of the HL161A and HL161B antibodies is shown in Table 7 and FIG. 6A to FIG. 6C.

TABLE 7
Change (%) in monkey IgG level by administration of HL161A and HL161B

| | | | HL161A | | HL161B | |
|---|---|---|---|---|---|---|
| Day | | Vehicle | 5 mg/kg | 20 mg/kg | 5 mg/kg | 20 mg/kg |
| 0 | day | 100.0 ± 0.0 | 100.0 ± 0.0 | 100.0 ± 0.0 | 100.0 ± 0.0 | 100.0 ± 0.0 |
| 0.5 | day | 99.0 ± 4.8 | 81.5 ± 1.8 | 101.5 ± 9.0 | 94.3 ± 5.4 | 96.2 ± 3.0 |
| 1 | day | 97.6 ± 15.9 | 67.2 ± 2.0 | 86.2 ± 11.9 | 83.9 ± 24.7 | 94.1 ± 7.0 |
| 2 | day | 97.8 ± 6.2 | 63.0 ± 3.3 | 74.2 ± 14 | 73.7 ± 11.3 | 71.7 ± 5.4 |
| 3 | day | 104.5 ± 13.1 | 61.8 ± 8.0 | 59.2 ± 11.0 | 68.3 ± 9.3 | 61.3 ± 6.0 |
| 4 | day | 100.9 ± 16.7 | 55.3 ± 4.1 | 45.1 ± 4.6 | 65.5 ± 12.2 | 44.3 ± 5.6 |
| 5 | day | 103.4 ± 12.5 | 60.8 ± 8.3 | 38.8 ± 4.9 | 65.0 ± 11.9 | 38.4 ± 3.7 |
| 6 | day | 113.3 ± 8.5 | 64.9 ± 11.7 | 39.7 ± 6.4 | 66.4 ± 11.3 | 39.0 ± 5.4 |
| 7 | day | 116.9 ± 23.3 | 58.7 ± 4.7 | 39.6 ± 5.4 | 61.4 ± 8.0 | 37.5 ± 3.2 |
| 7.5 | day | 92.4 ± 10.4 | 51.2 ± 7.2 | 38.7 ± 7.8 | 62.8 ± 8.3 | 39.3 ± 0.4 |
| 8 | day | 94.6 ± 8.7 | 48.0 ± 9.3 | 36.1 ± 5.3 | 60.7 ± 7.5 | 39.6 ± 5.9 |
| 9 | day | 117.6 ± 14.3 | 47.1 ± 4.4 | 33.8 ± 5.0 | 54.3 ± 6.9 | 31.0 ± 3.1 |
| 10 | day | 115.1 ± 16.7 | 49.7 ± 8.9 | 29.6 ± 5.8 | 53.6 ± 4.9 | 32.8 ± 4.3 |
| 11 | day | 114.6 ± 18.9 | 47.7 ± 4.2 | 30.4 ± 6.5 | 54.7 ± 4.2 | 39.9 ± 9.1 |
| 12 | day | 109.5 ± 13.1 | 51.7 ± 3.1 | 32.9 ± 5.7 | 56.5 ± 4.7 | 46.7 ± 9.1 |
| 13 | day | 111.1 ± 21.2 | 52.9 ± 6.4 | 35.7 ± 9.2 | 58.7 ± 3.8 | 45.4 ± 7.6 |
| 14 | day | 128.9 ± 17.7 | 54.7 ± 4.2 | 37.8 ± 9.6 | 60.6 ± 4.2 | 53.8 ± 11.3 |
| 17 | day | 95.6 ± 6.6 | 59.5 ± 10.3 | 40.2 ± 7.4 | 56.7 ± 4.4 | 48.4 ± 10.0 |
| 20 | day | 92.5 ± 8.4 | 62.4 ± 6.7 | 47.6 ± 8.9 | 61.8 ± 6.0 | 54.0 ± 9.5 |
| 23 | day | 107.1 ± 15.2 | 71.9 ± 6.5 | 61.8 ± 13.3 | 64.9 ± 4.4 | 56.8 ± 6.0 |
| 26 | day | 104.0 ± 5.6 | 77.7 ± 6.8 | 72.2 ± 22.4 | 70.8 ± 7.4 | 62.4 ± 5.8 |
| 29 | day | 102.4 ± 8.3 | 81.4 ± 6.7 | 77.9 ± 20.5 | 74.8 ± 5.1 | 65.4 ± 10.8 |

2) Analysis of Pharmacokinetic Profiles of HL161A and HL161B in Monkey Blood

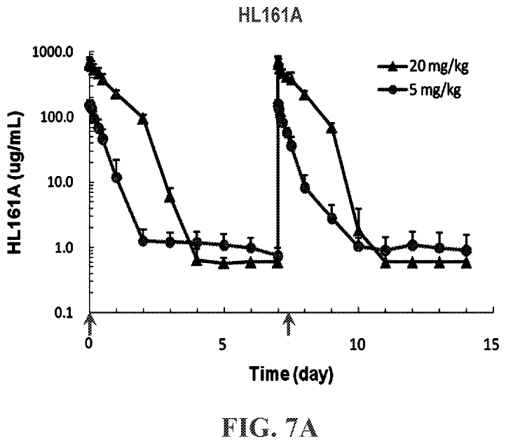

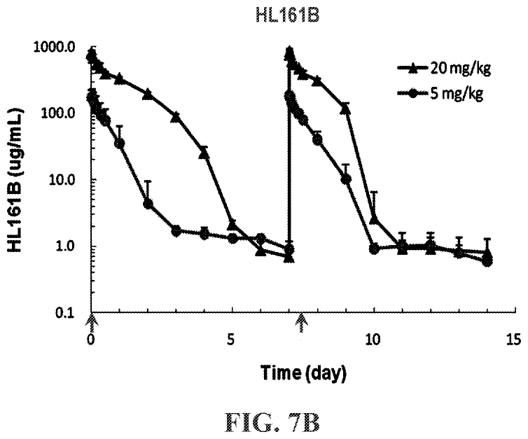

The time-dependent pharmacokinetic profiles (PK) of HL161A and HL161B after intravenous administration were analyzed by competitive ELISA. Specifically, a solution of 2 μg/mL of Neutravidin was prepared, and 100 μL of the solution was coated on each well of a 96-well plate, and then incubated at 4° C. for 18 hours. The plate was washed three time with 300 μL of wash buffer (0.05% Tween 20 containing 10 mM PBS, pH 7.4), and then each well was incubated with 1% BSA-containing PBS (pH 7.4) buffer at 25° C. for 2 hours. Biotinylated hFcRn was diluted with PBS to 1 μg/mL, and then 100 μL of the dilution was added to each well of the 96-well plate and incubated at 25° C. for 1 hour. Next, the plate was washed three times with 300 μL of wash buffer to remove unbound hFcRn, and then a standard sample (0.156-20 ng/mL) was added to each well and incubated at 25° C. for 2 hours. Next, the plate was washed three times with wash buffer, and 100 μL of a 1:10,000 dilution of detection antibody in PBS was added to each well and incubated at 25° C. for 1.5 hours. The plate was finally washed three times, and 100 μL of TMB solution was added to each buffer and incubated at room temperature for 5 minutes, after which 50 μL of 1 M sulfuric acid as a reaction stop solution was added to each well to stop the reaction. Next, the absorbance at 450 nm was measured with a microplate reader. The analysis results for pharmacokinetic profiles of HL161A and HL161B at varying doses are shown in Table 8 and FIG. 7A and FIG. 7B.

TABLE 8
Analysis results for pharmacokinetic profiles
of HL161A and HL161B at varying doses

| | | Cmax | AUC | T_{1/2} |
|---|---|---|---|---|
| Ab (Dose) | Day | (mg/ml) | (mg/ml · hr) | (hr) |
| HL161A | 0-7 | 157 ± 31 | 1,601 ± 501 | 6.9 ± 0.9 |
| (5 mg/kg) | 7-14 | 157 ± 25 | 1,388 ± 334 | 10.3 ± 2.8 |
| HL161A | 0-7 | 692 ± 138 | 13,947 ± 2,459 | 9.0 ± 0.6 |
| (20 mg/kg) | 7-14 | 724 ± 125 | 12,699 ± 2,114 | 7.6 ± 1.6 |
| HL161B | 0-7 | 178 ± 56 | 2,551 ± 1,356 | 7.9 ± 1.3 |
| (5 mg/kg) | 7-14 | 187 ± 9 | 2,772 ± 466 | 9.4 ± 0.5 |
| HL161B | 0-7 | 823 ± 38 | 21,867 ± 1,088 | 11.7 ± 1.0 |
| (20 mg/kg) | 7-14 | 868 ± 66 | 16,116 ± 1,501 | 6.8 ± 0.9 |

3) Analysis of Change in IgM and IgA Antibody Levels in Monkey Blood

ELISA analysis for measuring IgM and IgA levels in monkey blood was performed in a manner similar to the ELISA method for measuring IgG levels. Specifically, 100 μL of anti-monkey IgM antibody (Alpha Diagnostic, 70033) or IgA antibody (Alpha Diagnostic, 70043) was added to each well of a 96-well plate to a concentration of 2.0 g/mL, and then coated at 4° C. for 16 hours. The plate was washed three times with wash buffer (0.05% Tween-20 containing 10 mM PBS, pH 7.4), and then incubated with 1% BSA-containing PBS (pH 7.4) buffer at room temperature for 2 hours. The standard monkey IgM was analyzed at a concentration of 7.8-1,000 ng/mL, and IgA was analyzed at 15.6-2,000 ng/mL. The blood sample was diluted 10,000- or 20,000-fold in 1% BSA-containing PBS (pH 7.4) buffer, and the dilution was added to each well and incubated at room temperature for 2 hours. Next, the plate was washed three times with wash buffer, and then 100 μL of a 5,000-fold dilution of each of anti-monkey IgM secondary antibody (Alpha Diagnostic, 70031) and anti-monkey IgA secondary antibody (KPL, 074-11-011) was added to each well and allowed to react at room temperature for 1 hour. The plate was finally washed three times, and 100 μL of the substrate solution 3,3′,5,5′-tetramethylbenzidine (RnD, Cat. No: DY999) was added to each well and allowed to react at room temperature for 7 minutes. Next, 50 μL of 1.0 M sulfur solution (Samchun, Cat. No: S2129) was added to each well to stop the reaction. The absorbance of each well was measured with a 450 and 540 nm absorbance reader (MD, Model: VersaMax).

4) Analysis of Change in Albumin Levels in Monkey Blood

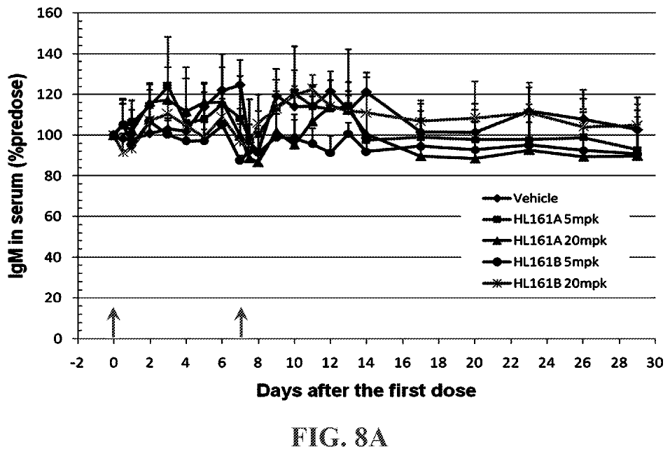

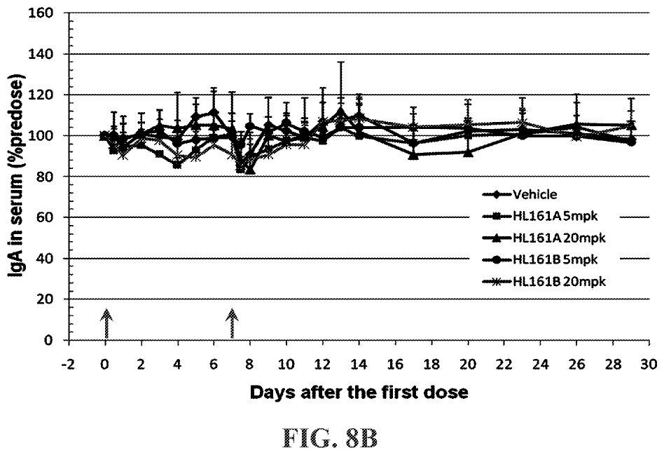

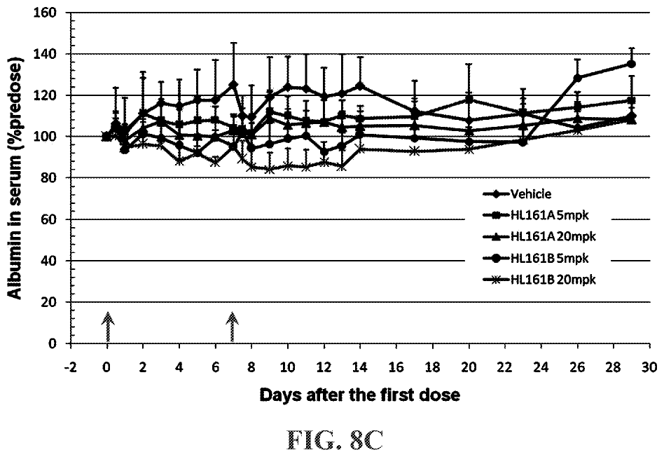

The analysis of a change in albumin levels in monkey blood was performed using a commercial ELISA kit (Assaypro, Cat. No: EKA2201-1). Briefly, monkey serum as a test sample was 4000-fold diluted, and 25 μL of the dilution was added to each well of a 96-well plate coated with an antibody capable of binding to monkey albumin. 25 μL of biotinylated monkey albumin solution was added to each well and incubated at 25° C. for 2 hours. The plate was washed three times with 200 μL of wash buffer, and then 50 μL of a 1:100 dilution of streptavidin-peroxidase conjugated antibody was added to each well and incubated at 25° C. for 30 minutes. The plate was finally washed three times, and then 50 μL of a substrate was added to each well and incubated at room temperature for 10 minutes. Next, 50 μL of a reaction stop solution was added to each well, and the absorbance at 450 nm was measured. Change (%) in monkey IgM, IgA, and albumin levels by administration of the HL161A and HL161B antibodies is shown in FIG. 8A to FIG. 8C.

5) Analysis of Blood Biochemical Levels and Urinary Components

Finally, blood biochemical analysis and urinary analysis by administration of the antibodies were performed using samples on Day 14 of the test. Blood biochemical markers, including aspartate aminotransferase (AST), alanine aminotransferase (ALT), alkaline phosphatase (ALP), creatine phosphokinase (CPK), total bilirubin (TBIL), glucose (GLU), total cholesterol (TCHO), triglyceride (TG), total protein (TP), albumin (Alb), albumin/globulin (A/G), blood urea nitrogen (BUN), creatinine (CRE), inorganic phosphorus (IP), calcium (Ca), sodium (Na), potassium (K) and chloride (Cl), were analyzed using the Hitachi 7180 system. In addition, markers for urinary analysis, including leukocyte (LEU), nitrate (NIT), urobilinogen (URO), protein (PRO), pH, occult blood (BLO), specific gravity (SG), ketone body (KET), bilirubin (BIL), glucose (GLU), and ascorbic acid (ASC), were analyzed using the Mission U120 system. Measured levels were generally in the normal level ranges of cynomolgus monkeys.

### Example 9: Assessment of RVT-1401 (HL161BKN) in Healthy Subjects Following Subcutaneous (SC) or Intravenous (IV) Administration

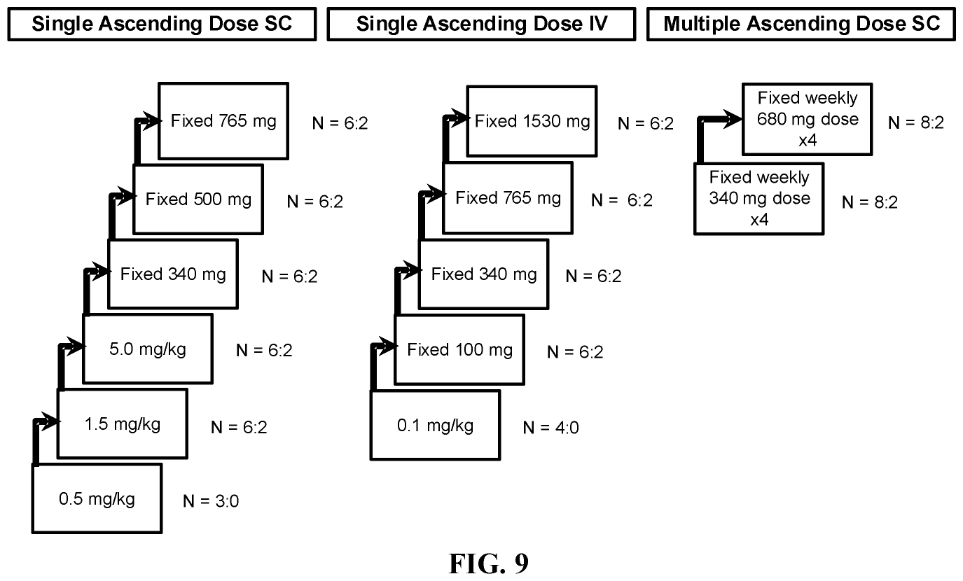

To assess the safety, tolerability, pharmacokinetics (PK), pharmacodynamics (PD), and immunogenicity of RVT-1401 (HL161BKN) following single (IV and SC) and multiple (SC) doses, RVT-1401 or placebo was administered to healthy subjects at the following doses (N=RVT-1401:placebo): 0.5 mg/kg SC (N=3:0); 1.5 mg/kg SC (N=6:2); 5.0 mg/kg SC (N=6:2); 340 mg SC (N=6:2); 500 mg SC (N=6:2); 765 mg SC (N=6:2); 0.1 mg/kg IV (N=4:0); 100 mg IV (N=6:2); 340 mg IV (N=6:2); 765 mg IV (N=6:2); 1530 mg IV (N=6:2); 340 mg weekly×4 (N=8:2); and 680 mg weekly×4 (N=8:2) (FIG. 9). Subject demographics are shown in Table 9.

TABLE 9
Subject demographics

| | SAD | | MAD | |
|---|---|---|---|---|
| | RVT-1401 | Placebo | RVT-1401 | Placebo |
| Demographic | (N = 61) | (N = 18) | (N = 16) | (N = 4) |
| Average Age | 36 | 36 | 37 | 36 |
| (yrs) [Range] | (19-55) | (18-55) | (21-48) | (32-40) |
| Average Weight | 79 | 71 | 76 | 82 |
| (kg) [Range] | (52-102) | (54-94) | (59-90) | (74-103) |
| Sex - Male (N %) | 57 (93%) | 13 (72%) | 16 (100%) | 4 (100%) |
| Sex - Female (N %) | 4 (7%) | 5 (28%) | 0 (0%) | 0 (0%) |
| Race - White (N %) | 54 (89%) | 15 (83%) | 14 (88%) | 4 (100%) |
| Race - Black or | 5 (8%) | 2 (11%) | 1 (6%) | 0 (0%) |
| African American | | | | |
| (N %) | | | | |
| Race - Asian (N %) | 0 (0%) | 1 (6%) | 1 (6%) | 0 (0%) |
| Race - Other (N %) | 2 (3%) | 0 (0%) | 0 (0%) | 0 (0%) |

### Results—

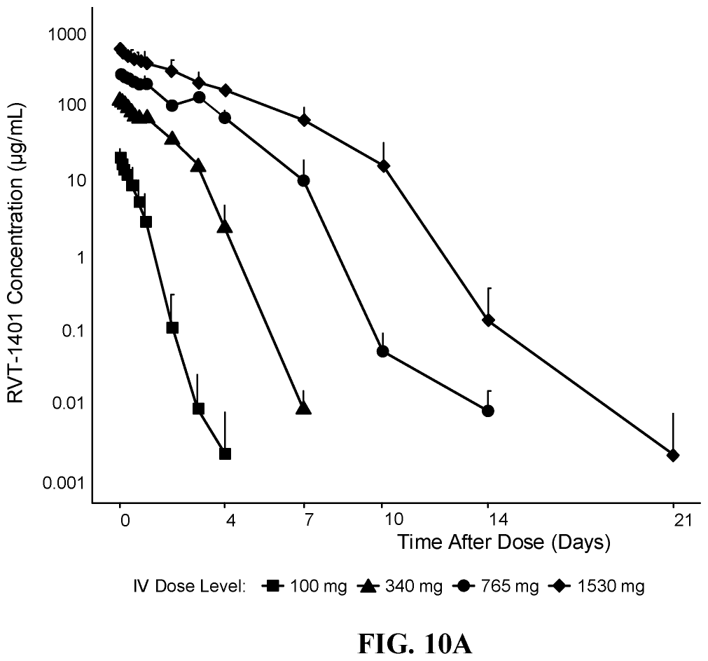

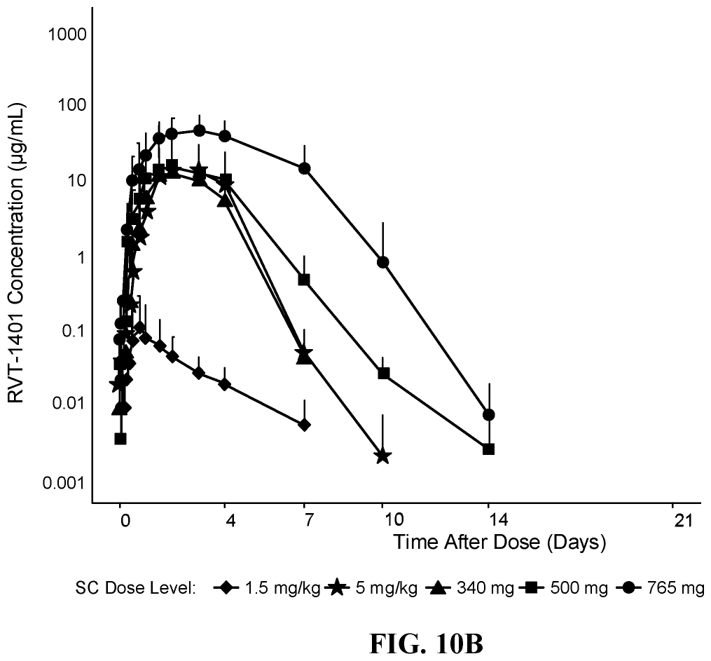

Pharmacokinetics (PK): Single dose PK (Cmax and AUC) following SC administration increased in a greater than dose proportional manner across the dose range of 1.5 mg/kg (equivalent mean: 127 mg) to 765 mg (fixed dose). A similar trend was observed following a 1-hour IV infusion across the dose range of 100 mg to 340 mg. Peak concentrations were observed between 1.5 to 4 days following SC administration of doses 340 mg and higher. Following IV infusion, serum terminal half-life (t_{1/2}) of RVT-1401 increased with dose. The dose-dependent half-life and greater than proportional increase in AUC was consistent with target-mediated drug disposition. A change in half-life with dose was observed following SC administration of doses above 340 mg, with t_{1/2} varying between 10 to 38 hours across all doses. The bioavailability of RVT-1401 administered subcutaneously was 11% and 23.5% after administration of 340 mg and 765 mg, respectively. Mean concentration-time profiles in healthy subjects following single dose IV and SC administration of RVT-1401 are shown in FIG. 10A and FIG. 10B. A summary of plasma PK parameters following single dose administration of RVT-1401 is shown in Tables 10 and 11.

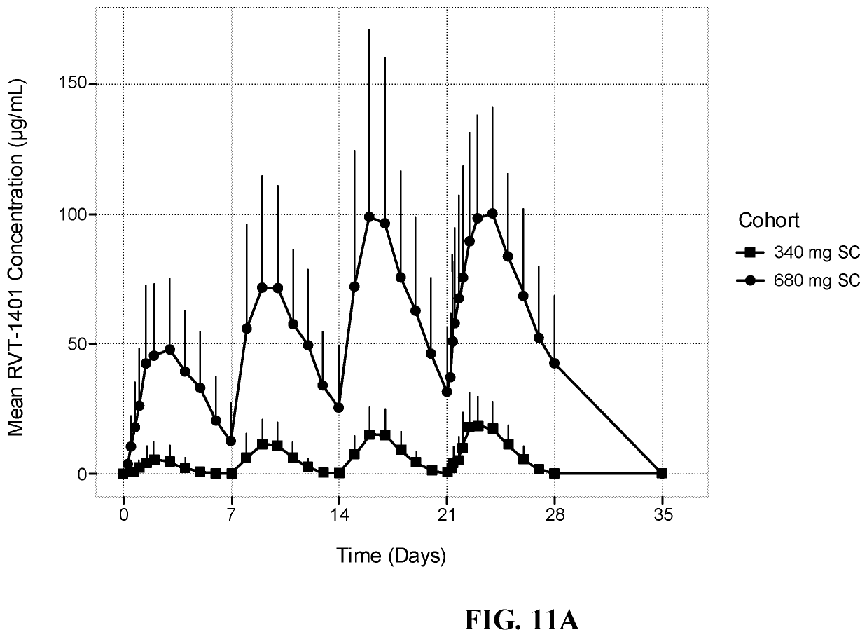

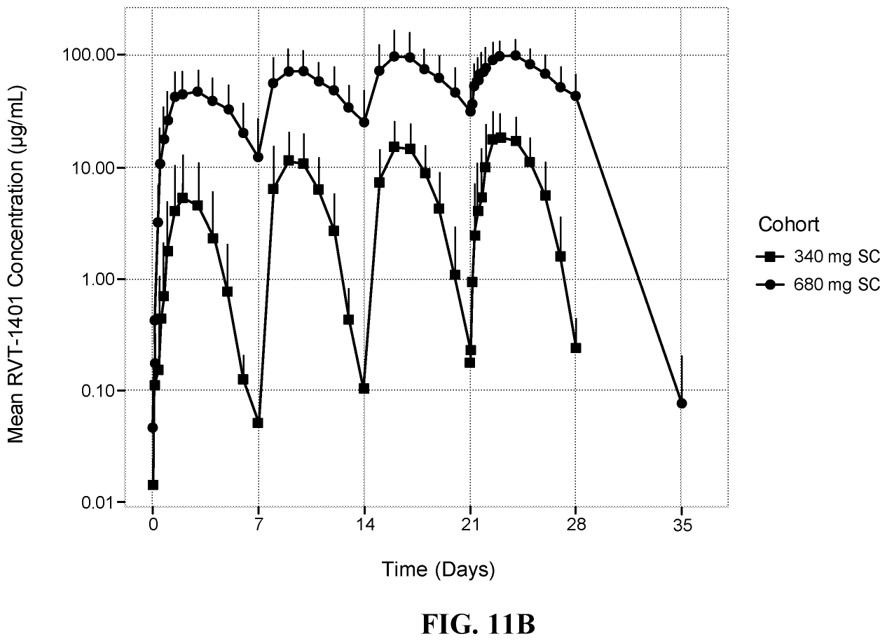

RVT-1401 was administered as a SC weekly injection of 340 mg or 680 mg for 4 weeks in the multidose cohorts. Following weekly SC administration of 340 mg, variability in Cmax and AUC(0-168) after the first dose of RVT-1401 was consistent with the single dose data. This inter-subject variability around Cmax and AUC(0-168) decreased after subsequent doses. Drug accumulation following weekly doses of 340 mg also showed large inter-subject variability, likely due to variability following the first dose. Repeat SC administration of 680 mg showed less inter-subject variability in exposure and had less accumulation after 4 weeks of dosing. Exposures (Cmax and AUC(0-168)) increased in more than a dose proportional manner when comparing Week 4 of the 340 mg and 680 mg SC doses. The increasing half-life and greater than proportional increase in AUC and Cmax with increasing dose was consistent with target-mediated drug disposition. Mean concentration-time profiles in healthy subjects following weekly SC administration of RVT-1401 at 340 mg or 680 mg are shown in FIG. 11A and FIG. 11B.

TABLE 10
Summary of PK parameters [geometric mean (% CV)] following
weight-based single SC administration of RVT-1401

| Dose | | | Weight¹ | AUC [symbol] | Cₘₐₓ | Tmax¹ | Half-Life |
|---|---|---|---|---|---|---|---|
| (mg/kg) | Route | N | (kg) | [symbol] | (ug/ml) | (Days) | (hrs) |
| 1.5 | SC | 5² | 84 | 5.20 | 0.08 | 0.75 | 46.4 |
| | | | (71, 102) | (69) | (170) | (0.33, 2.00) | (111) |
| 5.0 | SC | 6 | 75 | 833 | 12.8 | 2.5 | 12.0 |
| | | | (59, 97) | (60) | (54) | (2, 3) | (21.7) |

¹Median (Min, Max)
²One subject had no measurable concentrations following administration of 1.5 mg/kg, thus was not included in the PK parameters summary [symbol]
indicates data missing or illegible when filed

TABLE 11
Summary of PK parameters [geometric mean (% CV)] following
fixed dose single SC SC/IV administration of RVT-1401

| Dose | | | Weight¹ | AUC [symbol] | Cₘₐₓ | Tmax¹ | Half-Life² |
|---|---|---|---|---|---|---|---|
| (mg) | Route | N | (kg) | [symbol] | (ug/ml) | (Days) | (hrs) |
| 100 | IV | 6 | 92 | 211 | 19.9 | 0.04 | 5.30 |
| | | | (70, 101) | (51) | (25) | (0.04, 0.06) | (23) |
| 340 | IV | 6 | 71 | 3940 | 121 | 0.06 | 11.2 |
| | | | (69, 102) | (12) | (16) | (0.04, 0.08) | (29) |
| 765 | IV | 6 | 76 | 15500 | 273 | 1.75 | ND |
| | | | (58, 102) | (26) | (22) | (1.5, 8) | |
| 1530 | IV | 6 | 77 | 35100 | 530 | 1.5 | ND |
| | | | (54, 89) | (28) | (25) | (1, 6) | |
| 340 | SC | 6 | 83 | 453 | 7.58 | 1.5 | 14.6 (n = 5) |
| | | | (66, 97) | (333) | (275) | (1.5, 3) | (35) |
| 500 | SC | 6 | 74 | 323 | 4.26 | 2.5 | 21.6 (n = 5) |
| | | | (70, 91) | (626) | (661) | (1.5, 4) | (56) |
| 765 | SC | 6 | 75 | 4110 | 42.5 | 3 | 16.1 (n = 5) |
| | | | (52, 98) | (77) | (57) | (2, 4) | (14.5) |

¹Median (Min, Max)
²The 340, 500, and 765 mg SC cohorts each had one subject in which the terminal half-life could not be calculated due to insufficient number of points in the terminal phase
ND = Not determined at time of data cut-off [symbol]
indicates data missing or illegible when filed

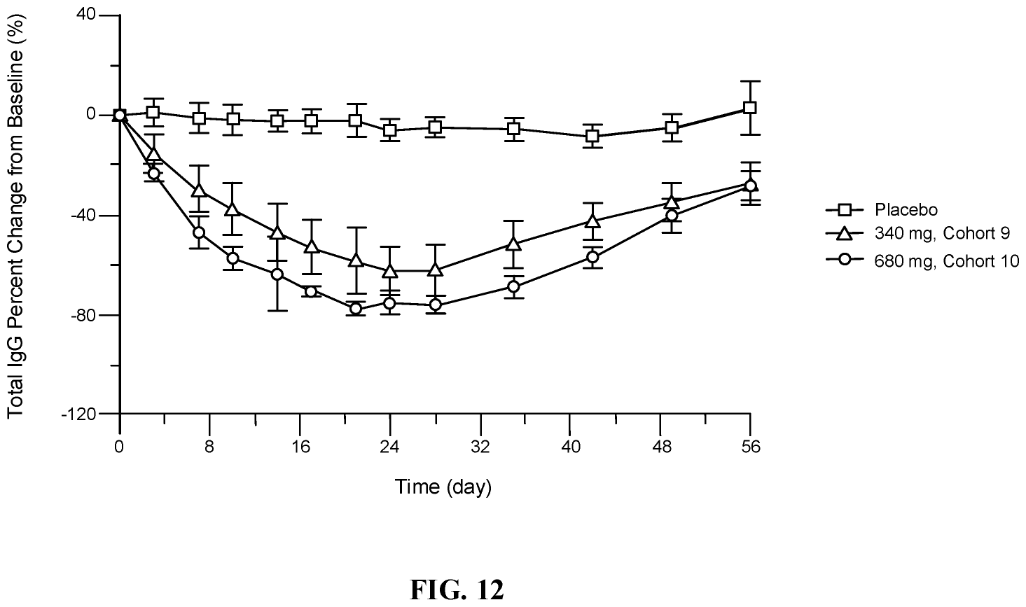

Primary Pharmacodynamics (PD): Dose-dependent reductions in IgG compared to baseline were observed following single dose SC and IV administration of RVT-1401. The time to nadir concentration for IgG was between 7 and 14 days following administration of RVT-1401. Recovery back to baseline was generally achieved within 56 days after the last dose. The highest percent IgG reduction following a single SC dose was 48% after administration of 765 mg. Following repeated dosing of RVT-1401, there was a cumulative reduction in IgG and albumin concentrations in both 340 mg and 680 mg cohorts. Following weekly SC administration of 680 mg, nadir concentrations for both IgG and albumin occurred prior to the last dose in most subjects, indicating that maximum reduction had been achieved by Week 4. A maximum reduction in IgG of 63% was observed following weekly SC administration of 340 mg for 4 weeks and 78% following weekly SC administration of 680 mg for 4 weeks. Five weeks after the last dose, mean (SD) IgG concentrations were 8.6 (2.5) g/L and 9.0 (2.0) g/L for the 340 mg and 680 mg cohorts, respectively, within 30% of the baseline value. Sustained IgG reduction (>35%) was maintained one month after the last dose, and no clinically relevant changes were observed in IgM or IgA. Serum IgG concentration-time profiles in healthy subjects following weekly SC administration of RVT-1401 at 340 mg or 680 mg are shown in FIG. 12. A summary of total IgG PD parameters following single dose administration of RVT-1401 is shown in Table 12. A summary of total IgG PD parameters following multiple dose administration of RVT-1401 is shown in Table 13.

TABLE 12
Summary of total IgG PD parameters [mean (SD)]
following single dose administration of RVT-1401

| | | | | | | Nadir | | Maximum | | Time to Nadir | |
|---|---|---|---|---|---|---|---|---|---|---|---|
| | | | | Baseline | | Concentration | | Reduction from | | Concentration¹ | |
| Dose | | Route | N | (g/L) | | (g/L) | | Baseline (%) | | (Days) | |
| 1.5 | mg/kg | SC | 6 | 10.8 | (1.2) | 9.3 | (1.5) | 14.0 | (5.25) | 14 | (10, 28) |
| 5.0 | mg/kg | SC | 6 | 10.3 | (1.8) | 7.1 | (1.4) | 31.3 | (5.07) | 10 | (7.0, 10) |
| 340 | mg | SC | 6 | 11.4 | (1.6) | 8.2 | (2.6) | 29.0 | (18.3) | 10.5 | (4, 21) |
| 500 | mg | SC | 6 | 12.0 | (1.8) | 7.6 | (1.1) | 36.3 | (9.0) | 10 | (7.0, 14) |
| 765 | mg | SC | 6 | 11.7 | (2.1) | 6.1 | (0.9) | 47.8 | (5.60) | 8.5 | (7.0, 14) |
| Placebo | | SC | 10 | 9.9 | (1.3) | 9.3 | (1.1) | 6.07 | (4.38) | 12 | (0.0, 84) |
| 100 | mg | IV | 6 | 9.6 | (1.1) | 8.4 | (1.1) | 12.8 | (4.18) | 3.5 | (2.0, 10) |
| 340 | mg | IV | 6 | 12.3 | (2.5) | 7.7 | (1.8) | 37.3 | (3.29) | 10 | (7.0, 10) |
| 765 | mg | IV | 6 | 14.4 | (1.7) | 6.2 | (0.9)² | 57.3 | (0.87)² | 10 | (10, 10)² |
| 1530 | mg | IV | 6 | 11.4 | (2.5) | 3.8 | (1.2) | 66.8 | (4.48) | 10 | (10, 14) |

| | | | | | | | | | | |
|---|---|---|---|---|---|---|---|---|---|---|
| Placebo | IV | 8 | 11.9 | (3.0) | 10.8 | (2.4) | 8.97 | (6.67) | 14 | (7.0, 84) |

¹Time to nadir is relative to administration of first dose; Median (Min, Max)
²N = 4

TABLE 13
Summary of total IgG PD parameters [mean (SD)]
following multiple dose administration of RVT-1401

| | | Body | | Nadir | Maximum | Time in Nadir |
|---|---|---|---|---|---|---|
| Dose | | Weight² | Baseline | Concentration | Reduction from | Concentration^{2,3} |
| (mg) | N | (kg) | (g/L) | (g/L) | Baseline (%) | (Days) |
| 340 | 8 | 80.0 | 11.8 | 4.4¹ | 62.6¹ | 24¹ |
| | | (66.8, 84.4) | (2.7) | (2.2) | (10.7) | (21, 28) |
| 680 | 8 | 70.9 | 12.6 | 2.8 | 78.4 | 21 |
| | | (59.2, 90.3) | (2.8) | (0.9) | (2.36) | (21, 24) |
| Placebo⁴ | 4 | 76.1 | 10.5 | 9.3 | 11.2 | 33 |
| | | (75.4, 107) | (2.3) | (1.9) | (1.70) | (21, 42) |

¹N = 7 for PD as one subject discontinued prior to 4^{th} and final dose due to personal reasons
²Median (Min, Max)
³Time to nadir is relative to administration of first dose
⁴Placebo group is pooled from subjects receiving placebo from both treatment groups

Secondary PD: Dose-dependent reductions in albumin concentrations were observed following repeat dose administration of RVT-1401 at 340 mg or 680 mg. No adverse events (AEs) were associated with the observed decrease in albumin. In all subjects, mean serum albumin levels remained within normal limits (>3.5 g/dL) after weekly dosing at 340 mg. At 680 mg, albumin dropped below normal limits in all subjects but remained above 3.0 g/dL for the duration of dosing except in one subject (that subject's albumin reached a nadir of 2.6 g/dL on Day 22 and Day 25 but did not result in any clinical signs, symptoms, or adverse events). In all subjects, albumin levels were within normal limits within 4 weeks of the last dose for the 680 mg cohort. Across both cohorts, on average, subject albumin levels were within 90% of their baseline values 5 weeks after the last dose, indicating the reversibility of the effect of RVT-1401 on albumin.

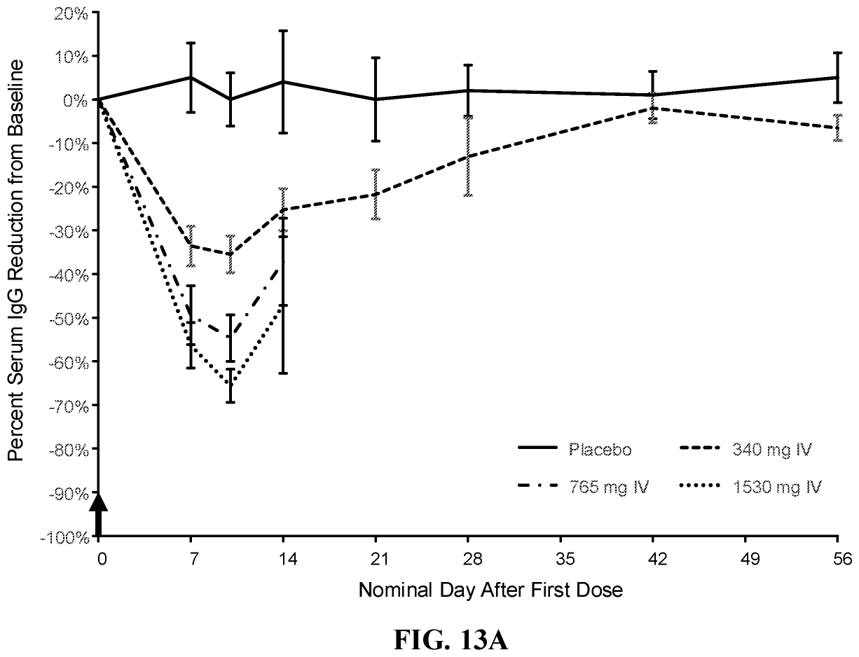

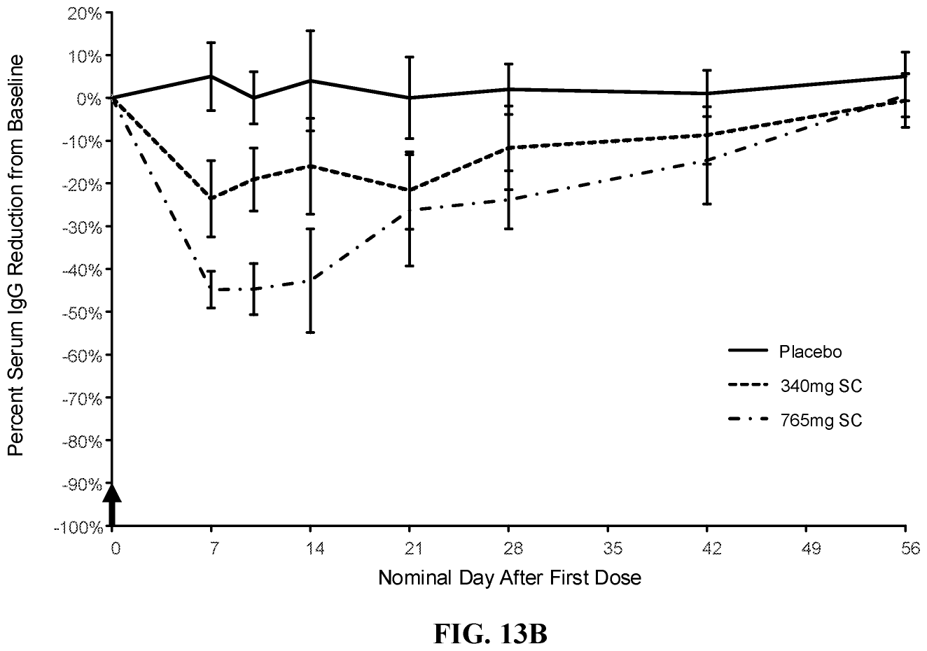

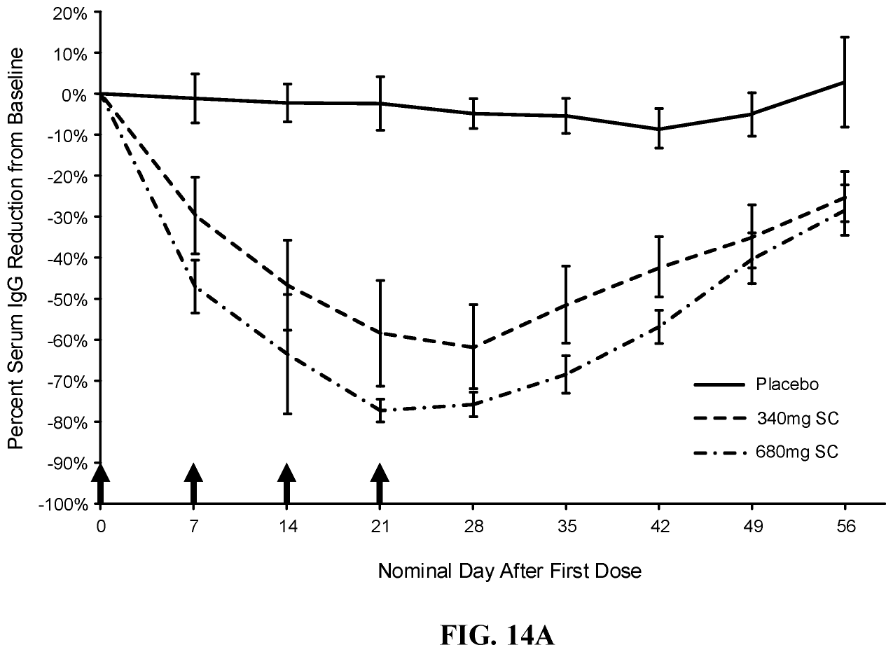

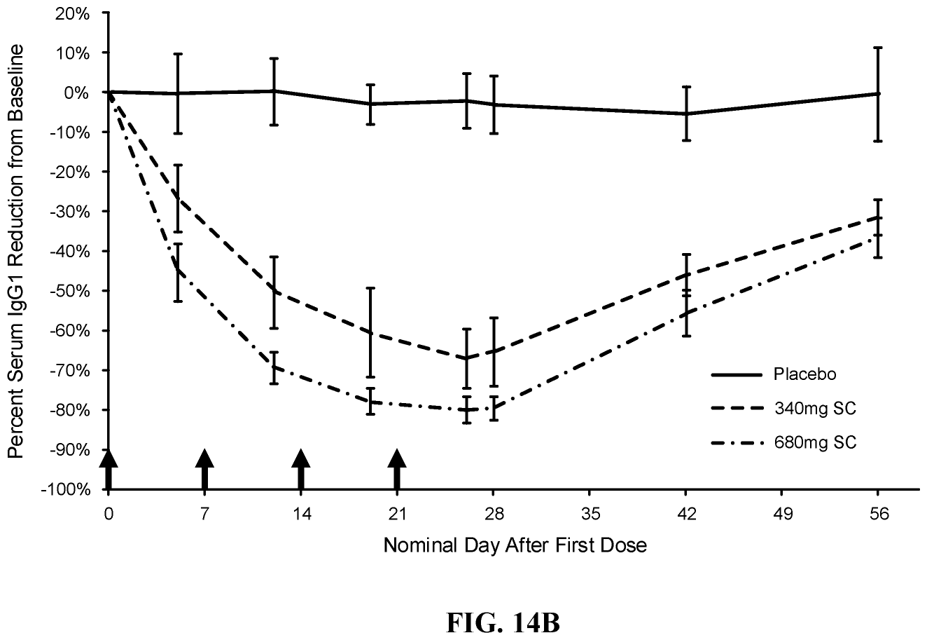

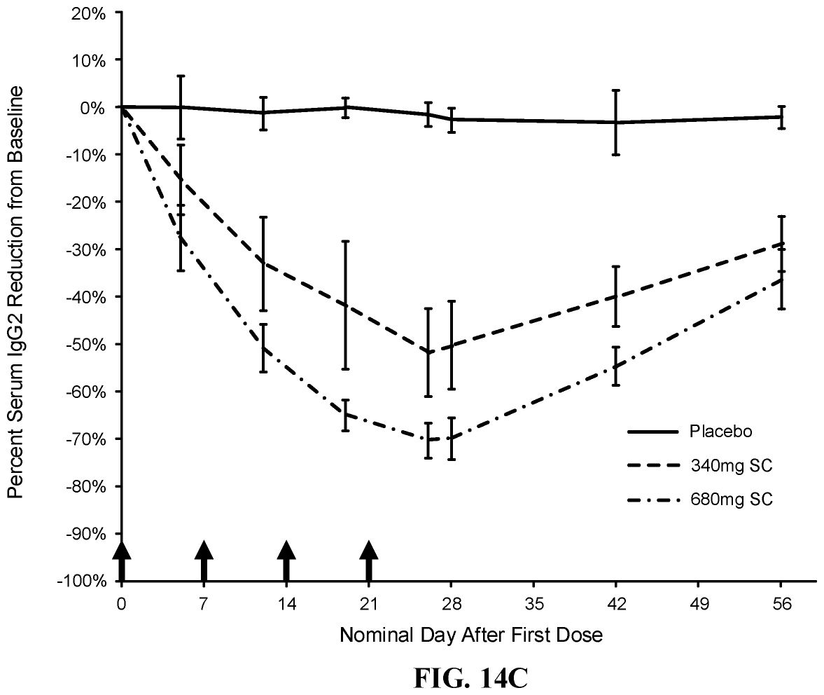

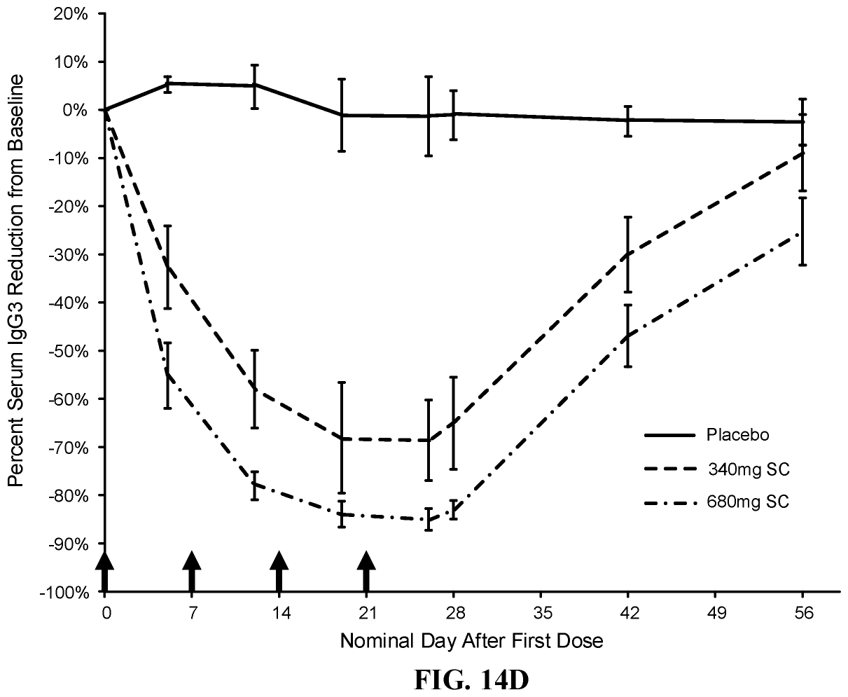

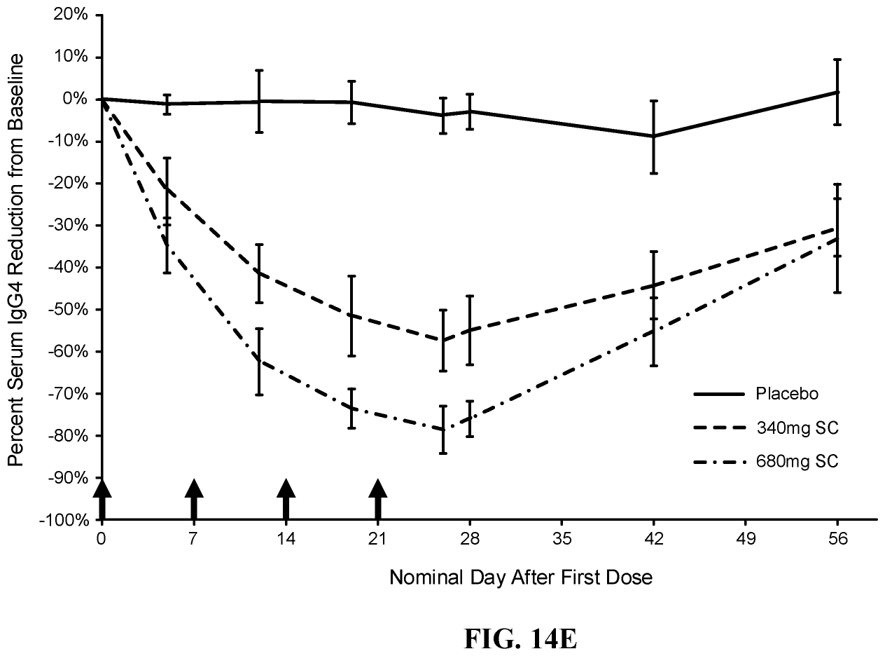

FIG. 13A shows percent (%) serum IgG reduction from baseline in healthy subjects following single dose IV administration of RVT-1401 (340 mg, 765 mg, 1530 mg) or placebo. FIG. 13B shows percent (%) serum IgG reduction from baseline in healthy subjects following single dose SC administration of RVT-1401 (340 mg, 765 mg) or placebo. FIG. 14A shows percent (%) serum IgG (total) reduction from baseline in healthy subjects following multiple dose SC administration of RVT-1401 (340 mg, 680 mg) or placebo. FIG. 14B shows percent (%) serum IgG1 reduction from baseline in healthy subjects following multiple dose SC administration of RVT-1401 (340 mg, 680 mg) or placebo. FIG. 14C shows percent (%) serum IgG2 reduction from baseline in healthy subjects following multiple dose SC administration of RVT-1401 (340 mg, 680 mg) or placebo. FIG. 14D shows percent (%) serum IgG3 reduction from baseline in healthy subjects following multiple dose SC administration of RVT-1401 (340 mg, 680 mg) or placebo. FIG. 14E shows percent (%) serum IgG4 reduction from baseline in healthy subjects following multiple dose SC administration of RVT-1401 (340 mg, 680 mg) or placebo. Maximum percent (%) serum IgG reduction from baseline for IgG subclasses (IgG1, IgG2, IgG3, and IgG4) is shown in Table 14.

TABLE 14
Maximum percent (%) serum IgG reduction for IgG subclasses

| | Dose | | | | |
|---|---|---|---|---|---|
| | Normalized | Maximum Percent (%) | | | |
| | to Weight** | Reduction from Baseline* | | | |
| Treatment | (mg/kg) | IgG1 | IgG2 | IgG3 | IgG4 |
| Placebo | 0 | 6.8 (6.8) | 6.9 (0.8) | 6.8 (2.2) | 12.1 (3.1) |
| 340 mg | 4.4 | 67.4 (8.2) | 50.7 (9.6) | 72 (9.0) | 58.1 (6.9) |
| | (4.0, 5.1) | | | | |
| 680 mg | 9.2 | 80.4 (2.9) | 70.6 (3.7) | 85.3 (2.3) | 78.7 (5.6) |
| | (7.5, 11.5) | | | | |

*Mean (SD);
**Mean (Min, Max).

Safety: RVT-1401 was generally well-tolerated, with no deaths or withdrawals due to adverse events (AEs), and all nonserious treatment emergent adverse events (TEAEs) of mild or moderate severity. Injection site reactions (erythema and/or swelling) were the most frequent TEAE for both RVT-1401 and placebo following SC administration (single and multiple dose). Injection site reactions were all mild in intensity and generally resolved within 1 to 4 hours after dosing. The frequency of injection site reactions was not dose-related and was similar for RVT-1401 and placebo. Other TEAEs seen in 3 or more subjects treated with either single or multiple doses of ≥340 mg/kg SC included headache and insomnia. Oropharyngeal pain and headache were the only TEAEs reported in 3 or more subjects following IV administration. All nonserious TEAEs in IV dose cohorts were mild to moderate in severity. Overall, there were no clinically relevant changes from baseline in vital signs, laboratory tests (including liver function tests), or on ECG following RVT-1401 SC or IV administration. There were no clinical signs or symptoms reported with the reductions in IgG or albumin in either the SC or IV cohorts. No headaches were observed following repeat SC injections of RVT-1401 at the 680 mg dose. Two serious AEs were reported, neither of which were related to RVT-1401.

Immunogenicity: The development of anti-drug antibodies (ADA) to RVT-1401 was assessed across all dosed cohorts following single (IV and SC formulations) and multiple (SC formulation) administrations of RVT-1401. Preliminary data showed an incidence of treatment-emergent ADA of 10.3% in RVT-1401-treated subjects and 6.7% in placebo-treated subjects across the single ascending dose cohorts, consistent with the high sensitivity of the ADA assay. Titers were considered low (≤1:16) and did not impact PK or PD. All ADAs resolved by the end of the monitoring period. There were no treatment-emergent ADA in either the 340 mg or 680 mg multiple ascending dose (MAD) cohorts.

### Example 10: A Non-Randomized, Open-Label Study of RVT-1401 for the Treatment of Patients with Warm Autoimmune Hemolytic Anemia (WAIHA)

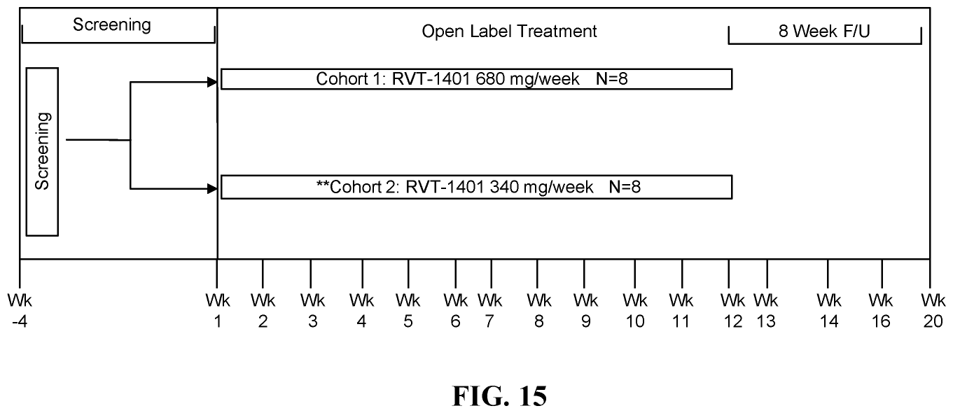

To assess the safety, tolerability, PK, PD, and efficacy of RVT-1401 (680 mg weekly and 340 mg weekly) in patients with warm autoimmune hemolytic anemia (WAIHA), two dosing regimens of RVT-1401 are assessed in a non-randomized, sequential, open-label study. Both dosing regimens involve once weekly subcutaneous (SC) injections: Dosing Regimen A (680 mg weekly for 12 weeks), and Dosing Regimen B (340 mg weekly for 12 weeks). Dosing Regimen A (680 mg weekly) is administered as two SC injections per week, and Dosing Regimen B (340 mg weekly) is administered as a single SC injection per week. The study design is shown in FIG. 15 and outlined below.

Study Design:

Screening—

Patients are diagnosed and screened for main inclusion/exclusion criteria (Table 15). Additional examples of inclusion/exclusion criteria are disclosed in NCT03226678, NCT04119050, and NCT03764618 (ClinicalTrials.gov), each of which is incorporated herein by reference for the disclosure of such criteria.

TABLE 15
Main inclusion/exclusion criteria
Criteria - Inclusion

| | |
|---|---|
| 1 | Male or female ≥18 years of age. |
| 2 | Diagnosis of primary or secondary WAIHA as documented by a positive direct |
| | antiglobulin test (DAT) specific for anti-IgG alone or anti-IgG plus C3d. |
| 3 | Secondary WAIHA may only include Stage 0 chronic lymphocytic leukemia (CLL) in |
| | which separate treatment is not indicated, nor anticipated to require active management |
| | for the duration of the study. |
| 4 | Have failed or not tolerated at least one prior WAIHA treatment regimen as per local |
| | standards (e.g., steroids, rituximab, azathioprine, cyclophosphamide, cyclosporine, |
| | mycophenolate mofetil (MMF), danazol, or vincristine). |
| 5 | Subjects with splenectomy ≥3 months from Day 1 who are up to date on vaccinations |
| | (based on age and local guidance) are allowed. |
| 6 | Haptoglobin < lower limit of normal (LLN) and lactate dehydrogenase (LDH) > upper |
| | limit of normal (ULN). |
| 7 | At Screening and Baseline, subject's hemoglobin level must be <10 g/dL and the subject |
| | must have documented symptoms related to anemia (e.g., weakness, dizziness, fatigue, |
| | shortness of breath, chest pain). |
| 8 | Kamofsky Performance status ≥60. |
| 9 | Subject's concurrent treatment for WAIHA may consist only of steroids (stable dose for |
| | at least two weeks prior to Day 1), immunosuppressant therapy (azathioprine, MMF, or |
| | cyclosporine) that has been at a stable dose for at least four weeks prior to Day 1, or |
| | erythropoietin (stable dose for at least 6 weeks prior to Day 1). [Note: starting doses of |
| | WAIHA therapy must be maintained throughout the study except in the case of a rescue |
| | medication as per local standards for safety. Steroid taper down to 10 mg/day will be |
| | allowed for subjects who achieve response for at least 2 weeks.] |

Criteria - Exclusion

| | |
|---|---|
| 1 | Subjects with other types of AIHA (e.g., cold antibody AIHA, cold agglutinin syndrome, |
| | mixed type AIHA, or paroxysmal cold hemoglobinuria). |
| 2 | Subjects requiring more than 2 units of RBC per week in the 2 weeks prior to Screening |
| | and Baseline. |
| 3 | Use of rituximab, any monoclonal antibody for immunomodulation, or proteasome |
| | inhibitor, within the past 3 months prior to Screening. |
| 4 | Immunoglobulins given by SC, IV (IVIG), or intramuscular route, or |
| | plasmapheresis/plasma exchange (PE) within 60 days before Screening. |
| 5 | Total IgG level <6 g/L (at Screening). |
| 6 | Absolute neutrophil count <1000 cells/mm³ (at Screening). |
| 7 | Albumin level <3.5 g/dL at Screening. |
| 8 | Known advanced liver disease including any diagnosis of cirrhosis of any stage. Non- |
| | alcoholic fatty liver disease (NAFLD) including non-alcoholic steatohepatitis (NASH) is |
| | allowable if there has been a recent (within 6 months) normal ultrasound, CT, or MRI. If |
| | the ultrasound, CT, or MRI demonstrate fatty changes alone, the subject may be enrolled |
| | if s/he has a normal range fibroscan for liver fibrosis. |
| 9 | AST or ALT ≥1.5x ULN at Screening. The subject may only be enrolled if s/he has a |
| | recent (within 6 months) normal ultrasound, CT, or MRI. If the ultrasound, CT, or MRI |
| | demonstrate fatty changes alone, the subject may be enrolled if s/he has a normal range |
| | fibroscan for liver fibrosis. |
| 10 | Subject has any laboratory abnormality (at Screening) that is clinically significant, has |
| | not resolved at Baseline, and could jeopardize or would compromise the subject's ability |
| | to participate in the study. |
| 11 | Medical history of primary immunodeficiency, T-cell or humoral, including common |
| | variable immunodeficiency. |
| 12 | Have an active infection, a recent serious infection (i.e., requiring injectable antimicrobial |
| | therapy or hospitalization) within the 8 weeks prior to Screening. |
| 13 | History of or known infection with human immunodeficiency virus (HIV), hepatitis B |
| | virus (HBV), or Mycobacterium tuberculosis: |
| | Subjects must have negative test results for HBV surface antigen, HBV core antibody, |
| | HIV 1 and 2 antibodies, and a negative QuantiFERON-TB Gold test at Screening. |
| | Subjects with an indeterminate QuantiFERON-TB Gold test result will be allowed one |
| | retest; if not negative on retesting, the subject will be excluded. |
| 14 | Infection with hepatitis C virus (HCV): |
| | Subjects must have a negative test result for HCV antibody. |
| | Subjects with a known history of HCV must have documented evidence of sustained |
| | virologic response that is consistent with cure of hepatitis C infection. This is defined as |
| | undetectable or unquantifiable HCV RNA at least 12 weeks after stopping HCV |
| | treatment (HCV Guidance: Recommendations for Testing, Managing, and Treating |
| | Hepatitis C; 2014-2018, AASLD and IDSA). This should be confirmed with a negative |
| | HCV RNA test at Screening. |
| 15 | Active malignancy or history of malignancy in the 3 years prior to Screening (exclusive |
| | of non-melanoma skin cancer and cervical cancer in situ). |
| 16 | Subject has any medical condition (acute or chronic illness) or psychiatric condition that |
| | could jeopardize or would compromise the subject's ability to participate in the study. |
| 17 | Body Mass Index (BMI) at Screening ≥40 kg/m². |
| 18 | Use of investigational drug within 60 days or 5 half-lives of the drug (whichever is |
| | longer) before Screening. |
| 19 | Subject has received a live vaccination within 2 weeks prior to the Baseline Visit; or |
| | intends to have a live vaccination during the course of the study or within 7 weeks |
| | following the final dose of study treatment. |
| 20 | History of sensitivity to any of the study treatments, or components thereof or a history of |
| | anaphylaxis (i.e., serious, life-threatening allergic reactions) that contraindicates |
| | participation. |
| 21 | Pregnant or lactating females as determined by positive serum or urine human chorionic |
| | gonadotropin test at Screening or Baseline. |
| 22 | QTcF interval >450 milliseconds for males and >470 milliseconds for females at |
| | Screening (a single repeat is allowed for eligibility determination). QTcF >480 msec in |
| | subjects with Bundle Branch Block. |
| 23 | Diagnosis of concomitant idiopathic thrombocytopenia purpura (ITP)/Evans syndrome |
| | with platelet count <100,000. |

Treatment—

Two cohorts of patients are enrolled in a non-randomized sequential approach. Patients are enrolled into Cohort 1 (680 mg weekly) first, followed by Cohort 2 (340 mg weekly). Following the initial dose at the Baseline Visit (Week 1, Day 1), study visits occur weekly throughout the treatment period. Patients receive RVT-1401 for 12 weeks (680 mg weekly or 340 mg weekly). The dosing regimens are expected to provide a sustained total IgG reduction of approximately 7580 and 6570 for Regimen A and Regimen B, respectively. It is also expected that the nadir IgG reduction is achieved by the 3^{rd} to 5^{th} dose (depending on dose studied) and maintained following the remaining doses before rising back to baseline over the next 6 to 8 weeks after stopping treatment.

Following the final dose at Week 12, visits occur weekly through Week 14 and then at Week 16 and Week 20. Safety, PK, PD, and clinical assessments are collected throughout the study. Each patient participates in the study for up to approximately 24 weeks: up to a 4-week screening period, a 12-week treatment period, and an 8-week follow up period. During and following treatment, primary, secondary, and exploratory endpoints are assessed up to Week 20 (Table 16).

TABLE 16
Primary, secondary, and exploratory endpoints
Primary

| | |
|---|---|
| 1 | Proportion of responders (defined as Hb level ≥10 g/dL with at least a ≥2 g/dL increase |
| | from baseline without rescue therapy or blood transfusions in the previous two weeks) at |
| | week 13 |
| 2 | Assessment of safety and tolerability by analysis of adverse event (AE) data and changes |
| | from baseline in vital signs, ECGs, and clinical laboratory values |

Secondary

| | |
|---|---|
| 1 | Change from baseline in Hb levels |
| 2 | Time to response |
| 3 | Change from baseline in hematocrit levels |
| 4 | Proportion of patients with Hb levels in the normal range at week 13 |
| 5 | Time to achieving Hb levels in the normal range |
| 6 | Change from baseline in FACIT-F score (fatigue) |
| 7 | Change from baseline in Medical Research Council (MRC) breathlessness scale |
| | (dyspnea) |
| 8 | Change from baseline in EQ-5D-3L score (health-related quality of life) |
| 9 | Change from baseline in levels of total IgG & IgG subclasses (I-IV) |
| 10 | Concentration of RVT-1401 pre-dose (Ctrough) |
| 11 | Change from baseline in LDH, bilirubin, and haptoglobin |
| 12 | Immunogenicity determined by change from pre-dose in anti-RVT-1401 antibodies, and |
| | characterization of any anti-RVT-1401 to confirm neutralization potential |

Exploratory

| | |
|---|---|
| 1 | Direct antiglobulin test (DAT) status by response |
| 2 | Proportion of subjects requiring rescue treatment (e.g., prednisone, dexamethasone, |
| | and/or blood transfusions) |
| 3 | Change from baseline in B cell phenotype |
| 4 | Change from baseline in anti-D, anti-Band 3, and/or anti-glycophorin antibodies |

Study Assessments and Procedures:

Physical Exams: A complete physical examination includes, at a minimum, assessment of the Cardiovascular, Respiratory, Gastrointestinal and Neurological systems and skin. Height is also measured and recorded at Screening only and weight at Screening and Baseline only. A brief physical examination includes, at a minimum, assessments of the skin, Respiratory and Cardiovascular systems, and abdomen (liver and spleen).

Vital Signs: Vital signs are measured in a supine position and include temperature, systolic and diastolic blood pressure, and pulse oximetry.

Electrocardiograms: Electrocardiograms (ECGs) are measured in a supine position. Twelve-lead ECGs are obtained during the study using an ECG machine that automatically calculates heart rate and measures PR, QRS, QT, and QTcF intervals.

Clinical Safety Laboratory Assessments: Hematology, clinical chemistry, urinalysis, and additional parameters to be tested by central laboratory are listed below in Table 17.

TABLE 17
Clinical safety laboratory assessments
Hematology

| Platelet Count | RBC Indices: | Automated WBC Differential: |
|---|---|---|
| Red Blood Cell (RBC)Count | Mean corpuscular | Neutrophils |
| | volume (MCV) | |
| White Blood Cell (WBC) Count (absolute) | Mean corpuscular | Lymphocytes |
| | hemoglobin (MCH) | |
| Reticulocyte Count | Mean corpuscular | Monocytes |
| | hemoglobin | |
| | concentration | |
| | (MCHC) | |
| Hemoglobin | | Eosinophils |
| Hemalocrit | | Basophils |
| Haptoglobin | | |

Clinical Chemistry

| | | | |
|---|---|---|---|
| Blood urea | Potassium | AST (SGOT) | Total (TBL) and |
| nitrogen (BUN) | | | direct bilirubin |
| Creatinine | Chloride | ALT (SGPT) | Uric Acid |
| Total Protein | Total carbon dioxide | Gamma glutamyltransferase | Albumin |
| | (CO₂) | (GGT) | |
| Sodium | Calcium (corrected) | Alkaline phosphatase (ALP) | Lactic acid |
| | | | dehydrogenase |
| | | | (LDH) |
| Serum | Immunoglobulin M | Immunoglobulin A | Immunoglobulin G |
| complement | (IgM) | (IgA) | (IgG) |
| (CH50, C3) | | | |
| HbA1c | | | |

Fasted labs

| | |
|---|---|
| Glucose (tested) | Insulin (fasted) |
| Week 1 and | Week 1 and Week 13 only |
| Week 13 only | |
| Lipid Panel | Total cholesterol |
| (fasted) | Triglycerides |
| Week 1, Week 13, | HDL cholesterol |
| Final Follow | LDL cholesterol (calculated using Martin-Hopkins equation) |
| up/Early | Cholesterol/HDL ratio |
| Withdrawal only | Non-HDL cholesterol (calculated) |
| | CRP/High sensitivity CRP |

Routine Urinalysis
Specific gravity, pH
glucose, protein, blood and ketones by dipstick
Microscopic examination (if blood or protein is abnormal)
Microalbumin/creatinine ratio at baseline, week 13, week 20 (if urine protein is abnormal)
Other tests
QuantiFERON ®-TB Gold
Viral Serology (HIV1/HIV2, Hepatitis B (HBsAg), Hepatitis B (Core antibody), Hepatitis C (Hep C
antibody]
Vaccine titers for: tetanus, diphtheria, Hepatitis A, Hepatitis B, Pneumococcal
FSH (as needed for confirmation of postmenopausal status)
Pregnancy Tests: serum test at screening, Week 20, and early withdrawal and urine dipstick pre-
dose at other timepoints. Positive urine tests should be confirmed with a serum test.

Pharmacokinetics (PK): Blood samples for PK analysis of RVT-1401 are collected at specified time points. The actual date and time of each blood sample collection is recorded.

Anti-Drug Antibody (ADA) and Neutralizing Antibody (NAb): Blood samples for ADA and NAb analysis are collected at specified time points. The actual date and time of each blood sample collection is recorded. Patients with treatment emergent positive results (change from baseline) for anti-RVT-1401 antibody at Week 20 are requested to return at approximately 6, 9, and 12 months post-dose for additional samples or until their result is no longer positive. However, for purposes of safety follow-up and database lock, participation ends at the Week 20 visit.

Pharmacodynamics (PD): Blood samples for PD analysis of RVT-1401 are collected at specified time points. The actual date and time of each blood sample collection is recorded. Pharmacodynamic markers include total IgG and differentiation by class (i.e., IgG subclasses (IgG1, IgG2, IgG3, and IgG4)).

Exploratory Biomarkers: Blood samples for exploratory biomarker analysis are collected at specified time points. The actual date and time of each blood sample collection is recorded. The timing of samples may be altered and/or samples may be obtained at additional time points to ensure thorough biomarker assessment. Exploratory biomarkers include B cell phenotype, DAT, anti-D antibodies, anti-Band 3 antibodies, and/or anti-glycophorin antibodies.

Interim Assessments:

Interim clinical safety laboratory assessments (hemoglobin and immunoglobulin G (IgG)) from two WAIHA patients treated with RVT-1401 at a dose of 680 mg weekly (Dosing Regimen A) are shown in Table 18.

TABLE 18
Interim clinical safety laboratory assessments - Hemoglobin and IgG

| | | | | | | | | |
|---|---|---|---|---|---|---|---|---|
| Baseline/ | | | | | | | | |
| Week 1 | Week 2 | Week 3 | Week 4 | Week 5 | Week 7 | Week 9 | Week 11 | Week 13 |

Patient 1

| | | | | | | | | | |
|---|---|---|---|---|---|---|---|---|---|
| Hemoglobin (g/dL) | 9.5 | 11.6 | 11.4 | 11.7 | 12.3 | 10.8 | 10.4 | 9.8 | 9.5 |
| IgG (g/L) | 8.2 | 4.35 | 2.95 | 2.07 | 2.02 | 1.88 | 2.12 | 2.46 | |

Patient 2

| | | | | | | |
|---|---|---|---|---|---|---|
| Hemoglobin (g/dL) | 6.4 | 7.1 | 7.1 | 7.4 | 7.8 | 7.7 |
| IgG (g/L) | 12.7 | 7.5 | 4.4 | 3.7 | 3.4 | N/A |

Both patients have a history of advanced WAIHA and failed at least 4 prior therapies for WAIHA. At the time of initiating open-label treatment with RVT-1401, both patients met all protocol eligibility criteria (Table 15).

At the time of the interim assessments, Patient 1 has completed 12 weeks of treatment and Patient 2 has completed 7 weeks of treatment. No injection site reactions have been reported for either patient.

Based on robust and quick onset of hemoglobin improvement in Patient 1 (i.e., an increase of more than 2 g/dL, which was observed by Week 2 and maintained for 4 weeks (Weeks 2 through 5)), the dose of prednisone and the dose of a second background WAIHA therapy for that patient were both decreased at Week 5. Without being bound by theory, these changes in doses of background medications may be related to the decrease in hemoglobin levels observed in Patient 1 starting at Week 7 and in the following weeks (Table 18).

Although the present disclosure has been described in detail with reference to the specific features, it will be apparent to those skilled in the art that this description is only for purposes of illustration and does not limit the scope of the present disclosure. Thus, the substantial scope of the present disclosure will be defined by the appended claims and equivalents thereof.

1. A method of treating or preventing warm autoimmune hemolytic anemia in a patient in need thereof, comprising administering to the patient: (i) a therapeutically effective amount of an anti-FcRn antibody or an antigen-binding fragment thereof, or (ii) a pharmaceutical composition comprising at least one pharmaceutically acceptable carrier and a therapeutically effective amount of an anti-FcRn antibody or an antigen-binding fragment thereof, wherein:
the antibody or antigen-binding fragment comprises a heavy chain variable region comprising an amino acid sequence of SEQ ID No: 27 (HCDR1), an amino acid sequence of SEQ ID No: 28 (HCDR2), and an amino acid sequence of SEQ ID No: 29 (HCDR3); and a light chain variable region comprising an amino acid sequence of SEQ ID No: 30 (LCDR1), an amino acid sequence of SEQ ID No: 31 (LCDR2), and an amino acid sequence of SEQ ID No: 32 (LCDR3); or
the antibody or antigen-binding fragment comprises a heavy chain variable region comprising an amino acid sequence of SEQ ID No: 21 (HCDR1), an amino acid sequence of SEQ ID No: 22 (HCDR2), and an amino acid sequence of SEQ ID No: 23 (HCDR3); and a light chain variable region comprising an amino acid sequence of SEQ ID No: 24 (LCDR1), an amino acid sequence of SEQ ID No: 25 (LCDR2), and an amino acid sequence of SEQ ID No: 26 (LCDR3); and
the therapeutically effective amount of the antibody or antigen-binding fragment is about 170 mg to about 1500 mg.

2. The method of claim 1, wherein the antibody or antigen-binding fragment comprises a heavy chain variable region comprising an amino acid sequence of SEQ ID No: 27 (HCDR1), an amino acid sequence of SEQ ID No: 28 (HCDR2), and an amino acid sequence of SEQ ID No: 29 (HCDR3); and a light chain variable region comprising an amino acid sequence of SEQ ID No: 30 (LCDR1), an amino acid sequence of SEQ ID No: 31 (LCDR2), and an amino acid sequence of SEQ ID No: 32 (LCDR3).

3. The method of claim 1, wherein the antibody or antigen-binding fragment comprises a heavy chain variable region comprising an amino acid sequence of SEQ ID No: 21 (HCDR1), an amino acid sequence of SEQ ID No: 22 (HCDR2), and an amino acid sequence of SEQ ID No: 23 (HCDR3); and a light chain variable region comprising an amino acid sequence of SEQ ID No: 24 (LCDR1), an amino acid sequence of SEQ ID No: 25 (LCDR2), and an amino acid sequence of SEQ ID No: 26 (LCDR3).

4. The method of claim 1, wherein the antibody or antigen-binding fragment comprises a heavy chain variable region comprising an amino acid sequence of SEQ ID No: 4 or SEQ ID No: 6; and a light chain variable region comprising an amino acid sequence of SEQ ID NO: 14 or SEQ ID No: 16.

5. The method of claim 1, wherein the antibody or antigen-binding fragment comprises a heavy chain variable region comprising an amino acid sequence of SEQ ID No: 2; and a light chain variable region comprising an amino acid sequence of SEQ ID No: 12.

6. The method of claim 1, wherein the antibody or antigen-binding fragment binds to FcRn with a K_{D} (dissociation constant) of 0.01 nM to 2 nM at pH 6.0 or pH 7.4.

7. The method of claim 6, wherein the K_{D} is measured by surface plasmon resonance (SPR).

8. The method of claim 1, wherein the antibody, antigen-binding fragment, or pharmaceutical composition is administered subcutaneously.

9. The method of claim 1, wherein the antibody, antigen-binding fragment, or pharmaceutical composition is administered as one or more subcutaneous injections.

10. The method of claim 9, wherein the antibody, antigen-binding fragment, or pharmaceutical composition is administered without intravenous administration prior to the one or more subcutaneous injections.

11. The method of claim 1, wherein the antibody, antigen-binding fragment, or pharmaceutical composition is administered once as a single dose or once weekly.

12. The method of claim 1, wherein the therapeutically effective amount of the antibody or antigen-binding fragment is 170 mg to 300 mg.

13. The method of claim 1, wherein the therapeutically effective amount of the antibody or antigen-binding fragment is 300 mg to 500 mg.

14. The method of claim 1, wherein the therapeutically effective amount of the antibody or antigen-binding fragment is 500 mg to 700 mg.

15. The method of claim 1, wherein the therapeutically effective amount of the antibody or antigen-binding fragment is 700 mg to 900 mg.

16. The method of claim 1, wherein the therapeutically effective amount of the antibody or antigen-binding fragment is 900 mg to 1100 mg.

17. The method of claim 1, wherein the therapeutically effective amount of the antibody or antigen-binding fragment is 1100 mg to 1300 mg.

18. The method of claim 1, wherein the therapeutically effective amount of the antibody or antigen-binding fragment is 1300 mg to 1500 mg.

19. The method of claim 1, wherein the antibody, antigen-binding fragment, or pharmaceutical composition is administered in combination with at least one additional therapeutic agent.
